(19) United States
(12) Patent Application Publication
(10) Pub. No.: US 2008/0255192 A1
(43) Pub. Date: Oct. 16, 2008

(54) Methods and Compositions for Selectin Inhibition

(75) Inventors: Neelu Kaila, Lexington, MA (US); Kristin Marie Janz, Arlington, MA (US); Adrian Huang, Lexington, MA (US); Alessandro Fabio Moretto, Somerville, MA (US); Patricia Ward Bedard, Foxboro, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/058,326

(22) Filed: Mar. 28, 2008

Related U.S. Application Data
(60) Provisional application No. 60/921,203, filed on Mar. 30, 2007.

(51) Int. Cl. A61K 31/47; C07D 215/20

(52) U.S. Cl. 514/312; 546/155

(57) ABSTRACT

The present teachings relate to novel compounds of formula I:

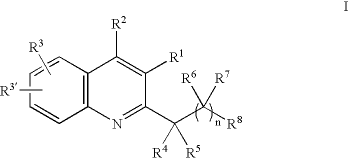

wherein the constituent variables are as defined herein. Compounds of the present teachings can act as antagonists of the mammalian adhesion proteins known as selecting. Methods for treating or preventing selectin-mediated disorders are provided, which include administration of these compounds in a therapeutically effective amount.

This application claims the benefit of U.S. Provisional Application No. 60/921,203 filed Mar. 30, 2007, the entire disclosure of which is incorporated by reference herein.

### FIELD

The present teachings relate to novel compounds that act as antagonists of the mammalian adhesion proteins known as selecting.

### BACKGROUND

During the initial phase of vascular inflammation, leukocytes and platelets in flowing blood decrease velocity by adhering to the vascular endothelium and by exhibiting rolling behavior. This molecular tethering event is mediated by specific binding of a family of calcium-dependent or “C-type” lectins, known as selectins, to ligands on the surface of leukocytes. There are also several disease states that can cause the deleterious triggering of selectin-mediated cellular adhesion, such as autoimmunity disorders, thrombotic disorders, parasitic diseases, and metastatic spread of tumor cells.

The extracellular domain of a selectin protein is characterized by an N-terminal lectin-like domain, an epidermal growth factor-like domain, and varying numbers of short consensus repeats. Three human selectin proteins have been identified, including P-selectin (formerly known as PADGEM or GMP-140), E-selectin (formerly known as ELAM-1), and L-selectin (formerly known as LAM-1). E-selectin expression is induced on endothelial cells by proinflammatory cytokines via its transcriptional activation. L-selectin is constitutively expressed on leukocytes and appears to play a key role in lymphocyte homing. P-selectin is stored in the alpha granules of platelets and the Weibel-Palade bodies of endothelial cells and therefore can be rapidly expressed on the surface of these cell types in response to proinflammatory stimuli. Selectins mediate adhesion through specific interactions with ligand molecules on the surface of leukocytes. Generally, the ligands of selectins are comprised, at least in part, of a carbohydrate moiety. For example, E-selectin binds to carbohydrates having the terminal structure:

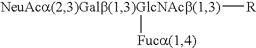

and also to carbohydrates having the terminal structures:

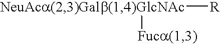

wherein R is the remainder of the carbohydrate chain. These carbohydrates are known blood group antigens and are commonly referred to as Sialyl Lewis x and Sialyl Lewis a, respectively. The presence of the Sialyl Lewis x antigen alone on the surface of an endothelial cell may be sufficient to promote binding to an E-selectin expressing cell. E-selectin also binds to carbohydrates having the terminal structures:

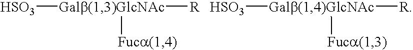

As with E-selectin, each selectin appears to bind to a range of carbohydrates with varying affinities. The strength of the selectin-mediated adhesive event (binding affinity) may also depend on the density and context of the selectin on the cell surface.

Structurally diverse glycoprotein ligands, including GlyCAM-1, CD34, ESL-1, and PSGL-1 can bind to selectins with apparent high affinity. PSGL-1 is a mucin-like homodimeric glycoprotein expressed by virtually all subsets of leukocytes and is recognized by each of the three selecting. However, PSGL-1 appears to be unique in that it is the predominant high affinity P-selectin ligand on leukocytes. High affinity P-selectin binding to PSGL-1 requires both an sLex-containing O-glycan and one or more tyrosine sulfate residues within the anionic N-terminus of the PSGL-1 polypeptide (see Somers, W. S. et al., Cell, 2000, 103: 467-479; Sako, D. et al., Cell, 1995, 82(2): 323-331; Pouyani, N. et al., Cell, 1995, 82(2): 333-343; and Wilkins, P. P. et al., J. Biol. Chem., 1995, 270(39): 22677-22680). L-Selectin also recognizes the N-terminal region of PSGL-1 and has similar sulfation-dependent binding requirements to that of P-selectin. The ligand requirements of E-selectin appear to be less stringent as it can bind to the sLex-containing glycans of PSGL-1 and other glycoproteins. Despite the fact that P-selectin knockout and P/E selectin double knockout mice show elevated levels neutrophils in the blood, these mice show an impaired DTH response and delayed thioglycolate-induced peritonitis (TIP) response (see Frenette, P. S. et al., Thromb Haemost, 1997, 78(1): 60-64). Soluble forms of PSGL-1 such as rPSGL-Ig have shown efficacy in numerous animal models (Lee Kumar, A. et. al., Circulation, 1999, 99(10): 1363-1369; Takada, M. et. al., J. Clin. Invest., 1997, 99(11): 2682-2690; and Scalia, R. et al., Circ Res., 1999, 84(1): 93-102).

In addition, P-selectin ligand proteins, and the genes encoding the same, have been identified. See U.S. Pat. No. 5,840,679. As demonstrated by P-selectin/LDLR deficient mice, inhibition of P-selectin represents a useful target for the treatment of atherosclerosis (see Johnson, R. C. et al., J. Clin. Invest., 1997, 99: 1037-1043). An increase in P-selectin expression has been reported at the site of atherosclerotic lesions, and the magnitude of the P-selectin expression appears to correlate with the lesion size. It is likely that the adhesion of monocytes, mediated by P-selectin, contributes to atherosclerotic plaque progression (see Molenaar, T. J. M. et al., Biochem. Pharmacol., 2003, (66): 859-866). Inhibition of P-selectin may also represent a useful target for other diseases or conditions, including, for example, thrombosis (Wakefield et al., Arterioscler Thromb Vasc Biol 28 (2008) 387-391; Myers et al., Thromb Haemost 97 (2007) 400-407), atherothrombosis (Fuster et al., Journal of the American College of Cardiology 46 (2005) 1209-1218), restenosis (Bienvenu et al., Circulation 103 (2001) 1128-1134), myocardial infarction (Furman et al., Journal of the American College of Cardiology 38 (2001) 1002-1006), ischemia reperfusion, Reynauld's syndrome, inflammatory bowel disease, osteoarthritis, acute respiratory distress syndrome, asthma (Romano, Treat Respir Med 4 (2005) 85-94), chronic obstructive pulmonary disease (Romano, Treat Respir Med 4 (2005) 85-94), emphysema, lung inflammation, delayed type hyper-sensitivity reaction (Staite et al., Blood 88 (1996) 2973-2979), idiopathic pulmonary fibrosis, cystic fibrosis, thermal injury, stroke, experimental allergic encephalomyelitis, multiple organ injury syndrome secondary to trauma, neutrophilic dermatosis (Sweet's disease), glomerulonephritis (Tianfu Wu, Arthritis & Rheumatism 56 (2007) 949-959), ulcerative colitis (Irving et al., European Journal of Gastroenterology & Hepatology 20 (2008) 283-289), Crohn's disease, necrotizing enterocolitis, cytokine-induced toxicity, gingivitis (Krugluger et al., J Periodontal Res 28: 145-151), periodontitis (Krugluger et al., J Periodontal Res 28: 145-151), hemolytic uremic syndrome, psoriasis (Friedrich et al., Archives of Dermatological Research 297 (2006) 345-351), systemic lupus erythematosus, autoimmune thyroiditis, multiple sclerosis, rheumatoid arthritis (Grober et al., J. Clin. Invest. 91 (1993) 2609-2619), Grave's disease (Hara et al., Endocr J. 43 (1996) 709-713), immunological-mediated side effects of treatment associated with hemodialysis or leukapheresis, granulocyte transfusion associated syndrome, deep vein thrombosis (Myers et al., Thromb Haemost 97 (2007) 400-407), post-thrombotic syndrome, unstable angina, transient ischemic attacks, peripheral vascular disease (e.g., peripheral arterial disease) (van der Zee et al., Clin Chem 52 (2006) 657-664), metastasis associated with cancer (McEver, Glycoconjugate Journal 14 (1997) 585-591), sickle syndromes (including but not limited to sickle cell anemia) (Blann et al., Journal of Thrombosis and Thrombolysis, 10.1007/s11239-007-0177-7 (Dec. 14, 2007)), organ rejection (graft vs. host), or congestive heart failure.

Given the role of selectins in numerous important biological processes, including inflammation and adhesion processes, it can be seen that there is a continuing need for new selectin inhibitors.

The present teachings provide compounds of formula I:

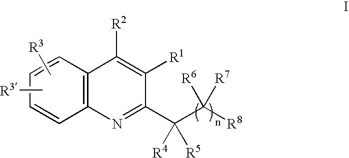

and pharmaceutically acceptable salts, hydrates, and esters thereof, wherein R¹, R², R³, R^{3′}, R⁴, R⁵, R⁶, R⁷, R⁸, and n are as defined herein.

The present teachings also relate to pharmaceutical compositions that include a pharmaceutically effective amount of one or more compounds of formula I (or pharmaceutically acceptable salts, hydrates, or esters thereof) and a pharmaceutically acceptable carrier or excipient. The present teachings also provide methods of making and using the compounds of formula I, and their pharmaceutically acceptable salts, hydrates, and esters. In some embodiments, the present teachings provide methods of treating mammals having conditions characterized by selectin-mediated intercellular adhesion processes, for example, by administering to the mammal an effective amount of one or more compounds of formula I (or their pharmaceutically acceptable salts, hydrates, and esters) to at least partially modulate selectin-mediated intracellular adhesion in a mammal.

### DETAILED DESCRIPTION

The present teachings provide compounds of formula I:

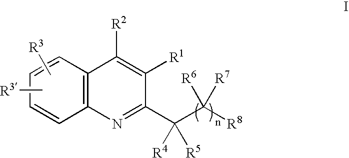

and pharmaceutically acceptable salts, hydrates, and esters thereof, wherein:

R¹ is —OR⁹, —C(O)R¹⁰, —C(O)OR⁹, —C(O)NR¹⁰R¹¹, —C(S)R¹⁰, —C(S)OR⁹, —C(S)NR¹⁰R¹¹, —C(NR¹⁰)R¹⁰, —C(NR¹⁰)NR¹⁰R¹¹, —NR¹⁰R¹¹, —NR¹¹C(O)R¹⁰, —NR¹¹C(O)NR¹⁰R¹¹, —NR¹¹C(NR¹⁰)NR¹⁰R¹¹, NR¹¹S(O)R¹⁰, or —NR¹¹S(O)ₘNR¹⁰R¹¹;

R² is —C(O)OR⁹, —C(O)NR¹⁰R¹¹, or a carboxylic acid bioisostere;

R³ and R^{3′} independently are H, —CN, —NO₂, halogen, —OR⁹, —NR¹⁰R¹¹, —S(O)ₘR¹⁰, —S(O)ₘOR⁹, —S(O)ₘNR¹⁰R¹¹, —C(O)R¹⁰, —C(O)OR⁹, —C(O)NR¹⁰R¹¹, —C(S)R¹⁰, —C(S)OR⁹, —C(S)NR¹⁰R¹¹, —C(NR¹⁰)NR¹⁰R¹¹, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₄ aryl group, the 3-14 membered cycloheteroalkyl group, and the 5-14 membered heteroaryl group optionally is substituted with 1-4-Z-R¹² groups; or

alternatively, R³ and R^{3′}, together with the carbon atoms to which each is attached, can form a C₄₋₁₄ cycloalkyl group, a C₆₋₁₄ aryl group, a 4-14 membered cycloheteroalkyl group, or a 5-14 membered heteroaryl group, wherein each of the C₄₋₁₄ cycloalkyl group, the C₆₋₁₄ aryl group, the 4-14 membered cycloheteroalkyl group, and the 5-14 membered heteroaryl group optionally is substituted with 1-4-Z-R¹² groups;

R⁴ and R⁵ independently are H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups; or

alternatively, R⁴ and R⁵, together with their respective common carbon atom, form a C₃₋₁₄ cycloalkyl group, a C₆₋₁₄ aryl group, a 3-14 membered cycloheteroalkyl group, or a 5-14 membered heteroaryl group, wherein each of the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;

R⁶ and R⁷, at each occurrence, independently are H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups; or

alternatively, R⁶ and R⁷, together with their respective common carbon atom, can form a C₃₋₁₄ cycloalkyl group, a C₆₋₁₄ aryl group, a 3-14 membered cycloheteroalkyl group, or a 5-14 membered heteroaryl group, wherein each of the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;

provided that at least one of R⁴ and R⁵, and R⁶ and R⁷, together with their respective common carbon atom, form a C₃₋₁₄ cycloalkyl group, a C₆₋₁₄ aryl group, a 3-14 membered cycloheteroalkyl group, or a 5-14 membered heteroaryl group, wherein each of the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;

R⁸ is a C₆₋₁₄ aryl group or a 5-14 membered heteroaryl group, wherein each of the C₆₋₁₄ aryl group and the 5-14 membered heteroaryl group optionally is substituted with 1-4-Z-R¹² groups;

R⁹, at each occurrence, independently is H, —C(O)R¹⁰, —C(O)NR¹⁰R¹¹, —C(S)R¹⁰, —C(S)NR¹⁰R¹¹, —C(NR¹⁰)R¹⁰, —C(NR¹⁰)NR¹⁰R¹¹, —S(O)ₘR¹⁰, —S(O)ₘNR¹⁰R¹¹, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;

R¹⁰ and R¹¹, at each occurrence, independently are H, —OH, —SH, —S(O)₂OH, —C(O)OH, —C(O)NH₂, —C(S)NH₂, —OC₁₋₁₀ alkyl, —C(O)—C₁₋₁₀ alkyl, —C(O)—OC₁₋₁₀ alkyl, —OC₆₋₁₄ aryl, —C(O)—C₆₋₁₄ aryl, —C(O)—OC₆₋₁₄ aryl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —C(O)NH—C₆₋₁₄ aryl, —S(O)ₘ—C₁₋₁₀ alkyl, —S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;

R¹², at each occurrence, independently is halogen, —CN, —NO₂, oxo, —O-Z-R¹³, —NR¹³-Z-R¹⁴, —N(O)R¹³-Z-R¹⁴, —S(O)ₘR¹³, —S(O)ₘO-Z-R¹³, —S(O)ₘNR¹³-Z-R¹⁴, —C(O)R¹³, —C(O)O-Z-R¹³, —C(O)NR¹³-Z-R¹⁴, —C(S)NR¹³-Z-R¹⁴, —Si(C₁₋₁₀ alkyl)₃, a C₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹⁵ groups;

R¹³ and R¹⁴, at each occurrence, independently are H, —OH, —SH, —S(O)₂OH, —C(O)OH, —C(O)NH₂, —C(S)NH₂, —OC₁₋₁₀ alkyl, —C(O)—C₁₋₁₀ alkyl, —C(O)—OC₁₋₁₀ alkyl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —S(O)ₘ—C₁₋₁₀ alkyl, —S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹⁵ groups;

R¹⁵, at each occurrence, independently is halogen, —CN, —NO₂, oxo, —OH, —NH₂, —NH(C₁₋₁₀alkyl), —N(C₁₋₁₀alkyl)₂, —S(O)ₘH, —S(O)ₘ—C₁₋₁₀ alkyl, —S(O)₂OH, l) —S(O)ₘ—OC₁₋₁₀ alkyl, —CHO, —C(O)—C₁₋₁₀ alkyl, —C(O)OH, —C(O)—OC₁₋₁₀ alkyl, —C(O)NH₂, —C(O)NH—C₁₋₁₀ alkyl, —C(O)N(C₁₋₁₀ alkyl)₂, —C(S)NH₂, —C(S)NH—C₁₋₁₀ alkyl, —C(S)N(C₁₋₁₀ alkyl)₂, —S(O)ₘNH₂, —S(O)ₘNH(C₁₋₁₀ alkyl), —S(O)ₘN(C₁₋₁₀ alkyl)₂, —Si(C₁₋₁₀ alkyl)₃, a C₁₋₁₀ alkyl group, a C₂₋₁₀ alkenyl group, a C₂₋₁₀ alkynyl group, a C₁₋₁₀ alkoxy group, a C₁₋₁₀ haloalkyl group, a C₃₋₁₄ cycloalkyl group, a C₆₋₁₄ aryl group, a 3-14 membered cycloheteroalkyl group, or a 5-14 membered heteroaryl group;

Z, at each occurrence, independently is a divalent C₁₋₁₀ alkyl group, a divalent C₂₋₁₀ alkenyl group, a divalent C₂₋₁₀ alkynyl group, a divalent C₁₋₁₀ haloalkyl group, or a covalent bond;

m, at each occurrence, independently is 0, 1, or 2; and

n is 0, 1, or 2.

In some embodiments, R¹ can be —OR⁹ or —NR¹⁰R¹¹, wherein R⁹ can be H, —C(O)R¹⁰, —C(O)NR¹⁰R¹¹, —C(S)R¹⁰, —C(S)NR¹⁰R¹¹, —S(O)ₘR¹⁰, —S(O)ₘNR¹⁰R¹¹, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group can be optionally substituted with 1-4-Z-R¹² groups, and R¹⁰, R¹¹, R¹², Z, and m are as defined herein. For example, R¹ can be —OH, —OC(O)R¹⁰, —OC(O)NR¹⁰R¹¹, —OS(O)ₘR¹⁰, —OS(O)ₘNR¹⁰R¹¹, or —NR¹⁰R¹¹. In certain embodiments, R¹ can be —OH, —OC(O)R¹⁰, or —NR¹⁰R¹¹. In particular embodiments, R¹ can be —OH.

In some embodiments, R² can be —C(O)OR⁹, wherein R⁹ is as defined herein. In certain embodiments, R⁹ can be H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group is independently and optionally substituted with 1-4-Z-R¹² groups, and Z and R¹² are as defined herein. For example, R² can be —C(O)OH.

In other embodiments, R² can be —C(O)NR¹⁰R¹¹, wherein R¹⁰ and R¹¹ are as defined herein. For example, R¹⁰ and R¹¹ independently can be H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups. In particular embodiments, R² can be —C(O)NH₂ or —C(O)NHR¹⁰, wherein R¹⁰ can be a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups.

In other embodiments, R² can be a carboxylic acid bioisostere, such as, but not limited to, an amide, a sulfonamide, a sulfonic acid, 3-hydroxy-4H-pyran-4-one, an imidazole, an oxazole, a thiazole, a pyrazole, a triazole, an oxadiazole, a thiadiazole, or a tetrazole, each of which optionally can be substituted (e.g., by a C₁₋₁₀ alkyl group, OH, etc.).

In some embodiments, compounds of the present teachings can be represented by formula Ia, formula Ib, formula Ic, formula Id, formula Ie, or formula If:

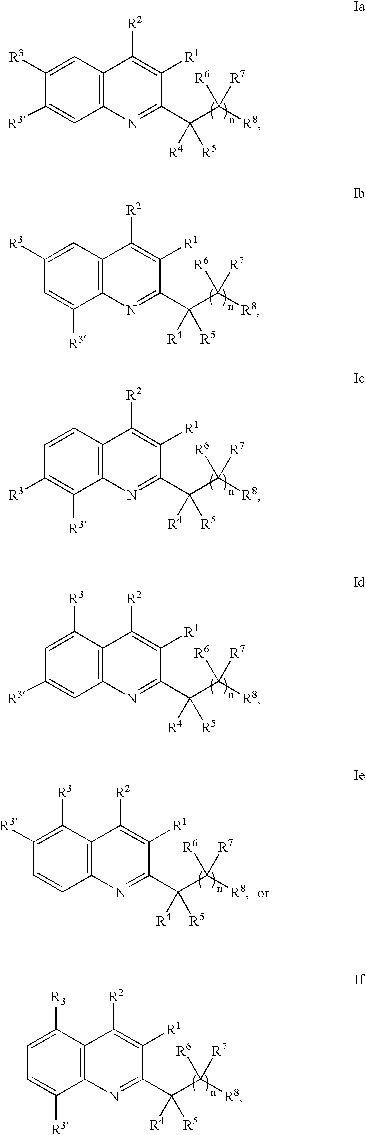

wherein R¹, R², R³, R^{3′}, R⁴, R⁵, R⁶, R⁷, R⁸, and n are as defined herein.

In some embodiments of the compounds represented by formula I, formula Ia, formula Ib, formula Ic, formula Id, formula Ie, or formula If, R³ and R^{3′} independently can be H, halogen, —OR⁹, —C(O)OR⁹, a C₁₋₁₀ alkyl group, a C₃₋₁₄ cycloalkyl group, a C₆₋₁₄ aryl group, or a 5-14 membered heteroaryl group, wherein each of the C₁₋₁₀ alkyl group, the C₃₋₁₄ cycloalkyl group, the C₆₋₁₄ aryl group, and the 5-14 membered heteroaryl group can be optionally substituted with 1-4-Z-R¹² groups, and Z and R¹² are as defined herein. In certain embodiments, R³ and R^{3′} independently can be H, F, Cl, Br, —OH, —O(C₁₋₆ alkyl), —COOH, a C₁₋₆ alkyl group, a C₃₋₁₀ cycloalkyl, a phenyl group, or a 5-10 membered heteroaryl group, wherein each of the C₁₋₆ alkyl group, the C₃₋₁₀ cycloalkyl group, the phenyl group, and the 5-10 membered heteroaryl group can be optionally substituted with 1-4-Z-R¹² groups, and Z and R¹² are as defined herein. For example, R³ and R^{3′} can independently be —O—(C₁₋₆ alkyl), wherein the C₁₋₆ alkyl group can be optionally substituted (e.g., —OCH₃, —OCH₂CH₃, —OCH(CH₃)₂, —OCH₂CH₂CH₃, —OC(CH₃)₃, and —OCF₃), an optionally substituted straight-chain or branched C₁₋₆ alkyl group (e.g. a methyl group, an ethyl group, a n-propyl group, an iso-propyl group, a n-butyl group, a sec-butyl group, a tert-butyl group, —CF₃, —C(CH₃)₂OH, —C(CF₃)(CH₃)OH, and —C(CF₃)₂OH), or an optionally substituted C₃₋₁₄ cycloalkyl group (e.g., a cyclopropyl group, a cyclobutyl group, a cyclopentyl group, a cyclohexyl group, and a cycloheptyl group). In some embodiments, R³ and R^{3′} can independently be H, —C(CH₃)₂OH, —C(CF₃)(CH₃)OH, or —C(CF₃)₂OH. In some embodiments, R³ can be H and R³ can be —C(CF₃)₂OH. In other embodiment, R³ can be —C(CF₃)₂OH and R³ can be H. In other embodiments, R³ and R^{3′} can both be H. In certain embodiments, R³ or R^{3′} can be a phenyl group or a thienyl group, each of which can be optionally substituted with 1-4-Z-R¹² groups, and Z and R¹² are as defined herein.

In other embodiments, R³ and R^{3′}, together with the carbon atoms to which each is attached, can form a C₄₋₁₄ cycloalkyl group or a 4-14 membered cycloheteroalkyl group, wherein each of the C₄₋₁₄ cycloalkyl group and the 4-14 membered cycloheteroalkyl group can be optionally substituted with 1-4-Z-R¹² groups, and Z and R¹² are as defined herein. Examples of cycloalkyl groups and cycloheteroalkyl groups include, but are not limited to, a cyclohexyl group and a piperidyl group, each of which can be optionally substituted with 1-4-Z-R¹² groups, and Z and R¹² are as defined herein. For example, R³ and R^{3′}, together with the carbon atoms to which they are attached, can form a cyclohexyl group. In some embodiments, compounds of the present teachings have formula Ig:

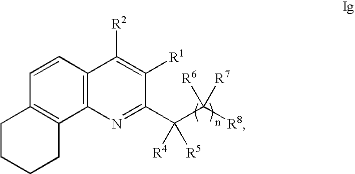

wherein R¹, R², R⁴, R⁵, R⁶, R⁷, R⁸ and n are as defined herein.

In some embodiments, R⁴ and R⁵ independently can be H or a C₁₋₆ alkyl group optionally substituted with 1-4-Z-R¹² groups, wherein Z and R¹² are as defined herein. In other embodiments, R⁴ and R⁵, together with their common carbon atom, can form a C₃₋₁₄ cycloalkyl group or a 3-14 membered cycloheteroalkyl group, wherein each of the C₃₋₁₄ cycloalkyl group and the 3-14 membered cycloheteroalkyl group can be optionally substituted with 1-4-Z-R¹² groups, and Z and R¹² are as defined herein. In certain embodiments, R⁴ and R⁵, together with their common carbon atom, can form a C₃₋₁₄ alkyl group optionally substituted with 1-4-Z-R¹² groups, and Z and R¹² are as defined herein. Examples of C₃₋₁₄ cycloalkyl groups include, but are not limited to, a cyclopropyl group, a cyclobutyl group, a cyclopentyl group, a cyclohexyl group, and a cycloheptyl group, each of which can be optionally substituted with 1-4-Z-R¹² groups, and Z and R¹² are as defined herein. In particular embodiments, R⁴ and R⁵, together with their common carbon atom, can form a cyclopropyl group or a cyclobutyl group.

In some embodiments, R⁶ and R⁷, at each occurrence, independently can be H or a C₁₋₆ alkyl group, wherein the C₁₋₆ alkyl group can be optionally substituted with 1-4-Z-R¹² groups, and Z and R¹² are as defined herein. In other embodiments, R⁶ and R⁷, together with their common carbon atom, can form a C₃₋₁₄ cycloalkyl group or a 3-14 membered cycloheteroalkyl group, each of which can be optionally substituted with 1-4-Z-R¹² groups, and Z and R¹² are as defined herein. For example, the C₃₋₁₄ cycloalkyl group can be a cyclopropyl group.

In some embodiments, at least one of R⁴ and R⁵, and R⁶ and R⁷, together with their respective common carbon atom, can form a C₃₋₁₄ cycloalkyl group, a C₆₋₁₄ aryl group, a 3-14 membered cycloheteroalkyl group, or a 5-14 membered heteroaryl group, wherein each of the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups, and Z and R¹² are as defined herein. In certain embodiments where R⁴ and R⁵ form a C₃₋₁₄ cycloalkyl group and n is 1, R⁶ and R⁷ independently can be H or a C₁₋₆ alkyl group optionally substituted with 1-4-Z-R¹² groups, wherein Z and R¹² are as defined herein. In other embodiments where R⁴ and R⁵ independently can be H or a C₁₋₆ alkyl group optionally substituted with 1-4-Z-R¹² groups and n is 1, R⁶ and R⁷ can form a C₃₋₁₄ cycloalkyl group, where Z and R¹² are as defined herein.

In some embodiments, R⁸ can be a C₆₋₁₄ aryl group optionally substituted with 1-4-Z-R¹² groups, and Z and R¹² are as defined herein. In certain embodiments, R⁸ can be a C₆₋₁₄ aryl group optionally substituted with a halogen, —O-Z-R¹³, a C₁₋₁₀ alkyl group, or a C₁₋₁₀ haloalkyl group, wherein Z and R¹³ are as defined herein. For example, R⁸ can be a phenyl group optionally substituted with F, Cl, Br, —OCH₃, —CH₃, —CF₃, and —OCF₃.

In some embodiments, R⁸ can be a 5-14 membered heteroaryl group optionally substituted with 1-4-Z-R¹² groups, and Z and R¹² are as defined herein. In certain embodiments, R⁸ can be a thienyl group optionally substituted with 1-4-Z-R¹² groups, and Z and R¹² are as defined herein. In particular embodiments, R⁸ can be an unsubstituted thienyl group.

In some embodiments of the compounds of the present teachings, n can be 0. In other embodiments, n can be 1.

For embodiments where n is 0, compounds of the present teachings can be represented by formula II:

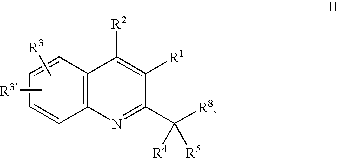

wherein R¹, R², R³, R^{3′}, R⁴, R⁵, and R⁸ are as defined herein. Certain compounds of these embodiments can be further represented by formula IIa, formula IIb, formula IIc, formula IId, formula IIe, or formula IIf:

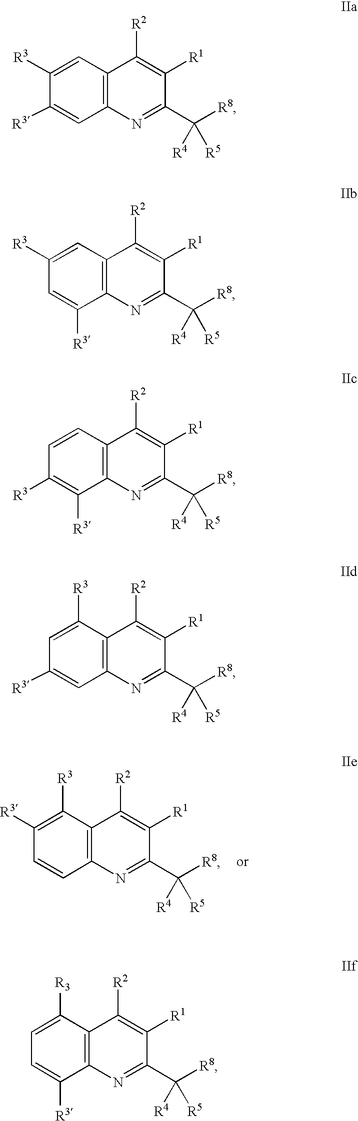

wherein R¹, R², R³, R^{3′}, R⁴, R⁵, and R⁸ are as defined herein.

In some embodiments of compounds represented by formula II, formula IIa, formula IIb, formula IIc, formula IId, formula IIe, or formula IIf, R³ and R^{3′}, together with the carbon atoms to which each is attached, form a C₄₋₁₄ cycloalkyl group or a 4-14 membered cycloheteroalkyl group, wherein each of the C₄₋₁₄ cycloalkyl group and the 4-14 membered cycloheteroalkyl group optionally is substituted with 1-4-Z-R¹² groups, and Z and R¹² are as defined herein. In some embodiments, R³ and R^{3′}, together with the carbon atoms to which each is attached, form a C₆ cycloalkyl group. For example, compounds of the invention can have a structure according to formula IIg:

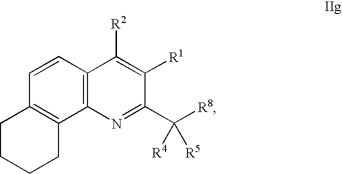

wherein R¹, R², R⁴, R⁵, and R⁶ are as defined herein.

In some embodiments of compounds represented by formula II, formula IIa, formula IIb, formula IIc, formula IId, formula IIe, formula IIf, or formula IIg, R⁴ and R⁵, together with their common carbon atom, can form a C₃₋₁₄ cycloalkyl group or a 3-14 membered cycloheteroalkyl group, wherein each of the C₃₋₁₄ cycloalkyl group and the 3-14 membered cycloheteroalkyl group can be optionally substituted with 1-4-Z-R¹² groups, and Z and R¹² are as defined herein. In certain embodiments, R⁴ and R⁵, together with their common carbon atom, can form a C₃₋₁₄ alkyl group optionally substituted with 1-4-Z-R¹² groups, and Z and R¹² are as defined herein. Examples of C₃₋₁₄ cycloalkyl groups include, but are not limited to, a cyclopropyl group, a cyclobutyl group, a cyclopentyl group, a cyclohexyl group, and a cycloheptyl group, each of which can be optionally substituted with 1-4-Z-R¹² groups, and Z and R¹² are as defined herein. In particular embodiments, R⁴ and R⁵, together with their common carbon atom, can form a cyclopropyl group or a cyclobutyl group.

In some embodiments of the compounds of the present teachings, R² can be C(O)OH and compounds of these embodiments can be represented by formula III, formula IIIa, or formula IIIb:

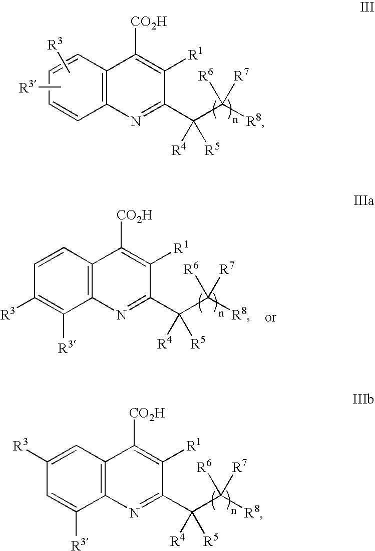

wherein R¹, R³, R^{3′}, R⁴, R⁵, R⁶, R⁷, R⁸, and n are as defined herein. In certain embodiments, n can be 0, and compounds of these embodiments can be further represented by formula IV, formula IVa, or formula IVb:

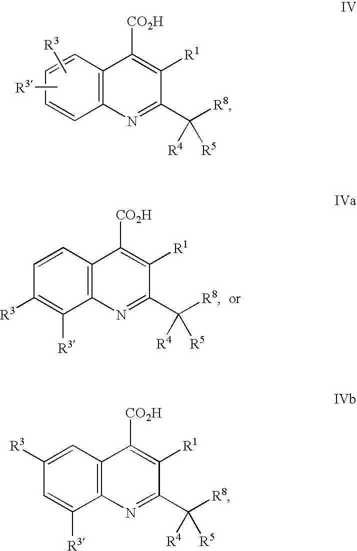

wherein R¹, R³, R^{3′}, R⁴, R⁵, and R⁸ are as defined herein

In some embodiments of compounds represented by formula III, formula IIIa, formula IIIb, formula IV, formula IVa, or formula IVb, R³ and R^{3′}, together with the carbon atoms to which each is attached, form a C₄₋₁₄ cycloalkyl group or a 4-14 membered cycloheteroalkyl group, wherein each of the C₄₋₁₄ cycloalkyl group and the 4-14 membered cycloheteroalkyl group optionally is substituted with 1-4-Z-R¹² groups, and Z and R¹² are as defined herein. In some embodiments, R³ and R^{3′}, together with the carbon atoms to which each is attached, form a C₆ cycloalkyl group. For example, compounds of the invention can have a structure according to formula IIIc or IVc:

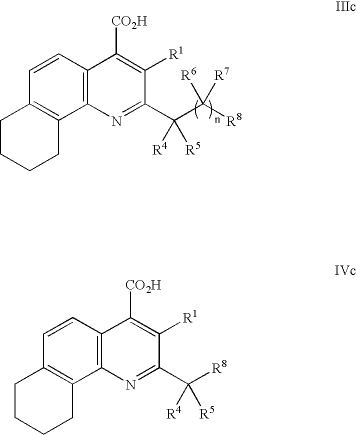

wherein R¹, R⁴, R⁵, R⁶, R⁷, R⁸, and n are as defined herein.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, “halo” or “halogen” refers to fluoro, chloro, bromo, and iodo.

As used herein, “oxo” refers to a double-bonded oxygen (i.e., ═O).

As used herein, “alkyl” refers to a straight-chain or branched saturated hydrocarbon group. Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl), and the like. In some embodiments, alkyl groups can be substituted with up to four substituents independently selected from -Z-R¹² group and -Z-R¹⁵ group, wherein Z, R¹², and R¹⁵ are as described herein. A lower alkyl group typically has up to 6 carbon atoms. Examples of lower alkyl groups include methyl, ethyl, propyl (e.g., n-propyl and isopropyl), and butyl groups (e.g., n-butyl, isobutyl, s-butyl, t-butyl).

As used herein, “alkenyl” refers to a straight-chain or branched alkyl group having one or more carbon-carbon double bonds. Examples of alkenyl groups include, but are not limited to, ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In some embodiments, alkenyl groups can be substituted with up to four substituents independently selected from -Z-R¹² group and -Z-R¹⁵ group, wherein Z, R¹², and R¹⁵ are as described herein.

As used herein, “alkynyl” refers to a straight-chain or branched alkyl group having one or more carbon-carbon triple bonds. Examples of alkynyl groups include, but are not limited to, ethynyl, propynyl, butynyl, pentynyl, and the like. The one or more carbon-carbon triple bonds can be internal (such as in 2-butyne) or terminal (such as in 1-butyne). In some embodiments, alkynyl groups can be substituted with up to four substituents independently selected from -Z-R¹² group and -Z-R¹⁵ group, wherein Z, R¹², and R¹⁵ are as described herein.

As used herein, “alkoxy” refers to an —O-alkyl group. Examples of alkoxy groups include, but are not limited to, methoxy, ethoxy, propoxy (e.g., n-propoxy and isopropoxy), t-butoxy groups, and the like. In some embodiments, the alkyl group in an —O-alkyl group can be substituted with up to four substituents independently selected from -Z-R¹² group and -Z-R¹⁵ group, wherein Z, R¹², and R¹⁵ are as described herein.

As used herein, “alkylthio” refers to an —S-alkyl group. Examples of alkylthio groups include, but are not limited to, methylthio, ethylthio, propylthio (e.g., n-propylthio and isopropylthio), t-butylthio groups, and the like. In some embodiments, the alkyl group in an —S-alkyl group can be substituted with up to four substituents independently selected from -Z-R¹² group and -Z-R¹⁵ group, wherein Z, R¹², and R¹⁵ are as described herein.

As used herein, “haloalkyl” refers to an alkyl group having one or more halogen substituents. Examples of haloalkyl groups include, but are not limited to, CF₃, C₂F₅, CHF₂, CH₂F, CCl₃, CHCl₂, CH₂C₁, C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.”

As used herein, “cycloalkyl” refers to a non-aromatic carbocyclic group including cyclized alkyl, alkenyl, and alkynyl groups, e.g., having from 3 to 14 ring carbon atoms and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Examples of cycloalkyl groups include, but are not limited to, cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like. In some embodiments, cycloalkyl groups can be substituted with up to four substituents independently selected from -Z-R¹² group and -Z-R¹⁵ group, wherein Z, R¹², and R¹⁵ are as described herein. In some embodiments, cycloalkyl groups can be substituted with one or more oxo groups.

As used herein, “heteroatom” refers to an atom of any element other than carbon or hydrogen and includes, for example, nitrogen (N), oxygen (O), sulfur (S), phosphorus (P), and selenium (Se).

As used herein, “cycloheteroalkyl” refers to a non-aromatic cycloalkyl group having 3-24 ring atoms that contains at least one ring heteroatom (e.g., 1-5) selected from O, N, and S, and optionally contains one or more (e.g., 1, 2, or 3) double or triple bonds. The cycloheteroalkyl group can be attached to the defined chemical structure at any heteroatom or carbon atom that results in a stable structure. One or more N or S atoms in a cycloheteroalkyl ring can be oxidized (e.g., morpholine N-oxide, thiomorpholine S-oxide, thiomorpholine S,S-dioxide). In some embodiments, nitrogen atoms of cycloheteroalkyl groups can bear a substituent, for example, a -Z-R¹² group and -Z-R¹⁵ group, wherein Z, R¹², and R¹⁵ are as described herein. Cycloheteroalkyl groups can also contain one or more oxo groups, such as phthalimide, piperidone, oxazolidinone, pyrimidine-2,4(1H,3H)-dione, pyridin-2(1H)-one, and the like. Examples of cycloheteroalkyl groups include, among others, morpholine, thiomorpholine, pyran, imidazolidine, imidazoline, oxazolidine, pyrazolidine, pyrazoline, pyrrolidine, pyrroline, tetrahydrofuran, tetrahydrothiophene, piperidine, piperazine, and the like. In some embodiments, cycloheteroalkyl groups can be optionally substituted with up to four substituents independently selected from -Z-R¹² group and -Z-R¹⁵ group, wherein Z, R¹², and R¹⁵ are as described herein.

As used herein, “aryl” refers to an aromatic monocyclic hydrocarbon ring system or a polycyclic ring system having an aromatic monocyclic hydrocarbon ring fused to at least one other aromatic hydrocarbon ring and/or non-aromatic carbocyclic or heterocyclic ring. In some embodiments, a monocyclic aryl group can have from 6 to 14 carbon atoms and a polycyclic aryl group can have from 8 to 14 carbon atoms. Any suitable ring position of the aryl group can be covalently linked to the defined chemical structure. In some embodiments, an aryl group can have only aromatic carbocyclic rings e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include, but are not limited to, benzodioxanyl, benzodioxolyl, chromanyl, indolinyl groups, and the like. In some embodiments, aryl groups can optionally contain up to four substituents independently selected from -Z-R¹² group and -Z-R¹⁵ group, wherein Z, R¹², and R¹⁵ are as described herein.

As used herein, “heteroaryl” refers to an aromatic monocyclic ring system containing at least 1 ring heteroatom selected from oxygen (O), nitrogen (N), and sulfur (S) or a polycyclic ring system where at least one of the rings present in the ring system is aromatic and contains at least 1 ring heteroatom. A heteroaryl group, as a whole, can have, for example, from 5 to 14 ring atoms and contain 1-5 ring heteroatoms. Heteroaryl groups include monocyclic heteroaryl rings fused to one or more aromatic carbocyclic rings, non-aromatic carbocyclic rings, and non-aromatic cycloheteroalkyl rings. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered monocyclic and 5-6 bicyclic ring systems shown below:

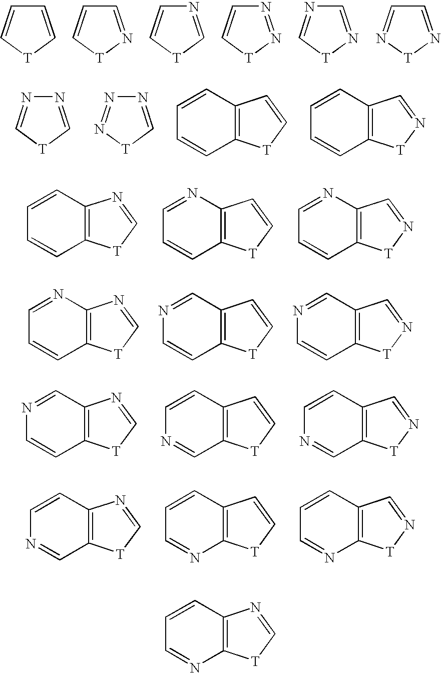

wherein T is O, S, NH, N-Z-R², or N-Z-R¹⁵, and Z, R¹², and R¹⁵ are defined as described herein. Examples of such heteroaryl rings include, but are not limited to, pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include, but are not limited to, 4,5,6,7-tetrahydroindolyl, tetrahydroquinolinyl, benzothienopyridinyl, benzofuropyridinyl groups, and the like. In some embodiments, heteroaryl groups can be substituted with up to four substituents independently selected from -Z-R¹² group and -Z-R¹⁵ group, wherein Z, R¹², and R¹⁵ are as described herein.

As used herein, “carboxylic acid bioisostere” refers to a substituent or group that has chemical or physical properties similar to that of a carboxylic acid moiety and that produces broadly similar biological properties to that of a carboxylic acid moiety. See generally, R. B. Silverman, The Organic Chemistry of Drug Design and Drug Action (Academic Press, 1992). Examples of carboxylic acid bioisosteres include, but are not limited to, amides, sulfonamides, sulfonic acids, phosphonamidic acids, alkyl phosphonates, N-cyanoacetamides, 3-hydroxy-4H-pyran-4-one, imidazoles, oxazoles, thiazoles, pyrazoles, triazoles, oxadiazoles, thiadiazoles, or tetrazoles, each of which optionally can be substituted (e.g., by a C₁₋₁₀ alkyl group, OH, etc.). Other examples of carboxylic acid bioisostere can include, but are not limited to, —OH and those shown below:

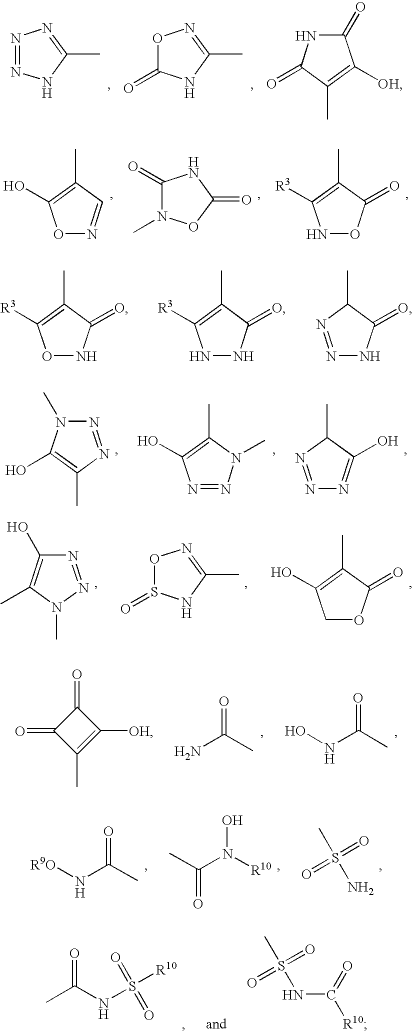

wherein R³, R⁹, and R¹⁰ are defined as herein.

Compounds of the present teachings can include a “divalent group” defined herein as a linking group capable of forming a covalent bond with two other moieties. For example, compounds described herein can include a divalent C₁₋₁₀ alkyl group, such as, for example, a methylene group.

At various places in the present specification, substituents of compounds are disclosed in groups or in ranges. It is specifically intended that the description include each and every individual subcombination of the members of such groups and ranges. For example, the term “C₁₋₁₀ alkyl” is specifically intended to individually disclose C₁, C₂, C₃, C₄, C₅, C₆, C₇, C₈, C₉, C₁₀, C₁-C₁₀, C₁-C₉, C₁-C₈, C₁-C₇, C₁-C₆, C₁-C₅, C₁-C₄, C₁-C₃, C₁-C₂, C₂-C₁₀, C₂-C₉, C₂-C₈, C₂-C₇, C₂-C₆, C₂-C₅, C₂-C₄, C₂-C₃, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉, and C₉-C₁₀ alkyl. By way of another example, the term “5-14 membered heteroaryl group” is specifically intended to individually disclose a heteroaryl group having 5, 6, 7, 8, 9, 10, 11, 12, 13, 14, 5-14, 5-13, 5-12, 5-11, 5-10, 5-9, 5-8, 5-7, 5-6, 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14 ring atoms. Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such optical isomers (enantiomers) and diastereomers (geometric isomers), as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Throughout the specification, structures may or may not be presented with chemical names. Where any question arises as to nomenclature, the structure prevails.

Also provided in accordance with the present teachings are prodrugs of compounds disclosed herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds as described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include, but are not limited to, acetate, formate and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

Ester forms of the compounds according to the present teachings include pharmaceutically acceptable esters known in the art which can be metabolized into the free acid form, such as a free carboxylic acid form, in a mammal body. Examples of suitable esters include, but are not limited to alkyl esters (e.g., alkyl of 1 to 10 carbon atoms), cycloalkyl esters (e.g., 3-10 carbon atoms), aryl esters (e.g., of 6-14 carbon atoms, including of 6-10 carbon atoms), and heterocyclic analogues thereof (e.g., of 3-14 ring atoms, 1-3 of which can be selected from oxygen, nitrogen, and sulfur heteroatoms) and the alcoholic residue can carry further substituents. In some embodiments, esters of the compounds disclosed herein can be C₁₋₁₀ alkyl esters, such as methyl ester, ethyl ester, propyl ester, isopropyl ester, butyl ester, isobutyl ester, t-butyl ester, pentyl ester, isopentyl ester, neopentyl ester, and hexyl ester, C₃₋₁₀ cycloalkyl esters, such as cyclopropyl ester, cyclopropylmethyl ester, cyclobutyl ester, cyclopentyl ester, and cyclohexyl ester, or aryl esters, such as phenyl ester, benzyl ester, and tolyl ester.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiinflammatory agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases, for example those in which the lung is the targeted organ, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically acceptable.

Compounds described herein can be administered parenterally or intraperitoneally. Solutions or suspensions of these compounds or pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal). Topical formulations that deliver compound(s) of the present teachings through the epidermis can be useful for localized treatment of inflammation, psoriasis, and arthritis.

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment, inhibition or prevention of a pathological condition or disorder in a mammal, for example, a human. The present teachings accordingly provide methods of treating or inhibiting a pathological condition or disorder by providing to a mammal a compound of the present teachings (or its pharmaceutically acceptable salt, hydrate, or ester) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder. As used herein, “therapeutically effective” refers to a substance or an amount that elicits a desirable biological activity or effect. As used herein, “treating” refers to partially or completely alleviating, inhibiting, and/or ameliorating the condition.

The present teachings further include use of the compounds disclosed herein and their pharmaceutically acceptable salts, hydrates, and esters as active therapeutic substances for the treatment, inhibition or prevention of a pathological condition or disorder in a mammal. In some embodiments, the pathological condition or disorder can be associated with selectin-mediated intracellular adhesion. Accordingly, the present teachings further provide methods of treating or preventing these pathological conditions and disorders using the compounds described herein.

In some embodiments, the present teachings provide methods of inhibiting selectin-mediated intracellular adhesion in a mammal that include administering to the mammal an effective amount of a compound of the present teachings or its pharmaceutically acceptable salt, hydrate, or ester. In certain embodiments, the present teachings provide methods of inhibiting selectin-mediated intracellular adhesion associated with a disease, disorder, condition, or undesired process in a mammal, that include administering to the mammal a therapeutically effective amount of a compound disclosed herein.

In some embodiments, the disease, disorder, condition, or undesired process can be infection, metastasis, an undesired immunological process, an undesired thrombotic process, or a disease or condition with an inflammatory component (e.g., cardiovascular disease, diabetes, or rheumatoid arthritis). In some embodiments, the disease, disorder, condition, or undesired process can be atherosclerosis, atherothrombosis, restenosis, myocardial infarction, ischemia reperfusion, Reynauld's syndrome, inflammatory bowel disease, osteoarthritis, acute respiratory distress syndrome, asthma, chronic obstructive pulmonary disease (COPD), emphysema, lung inflammation, delayed type hyper-sensitivity reaction, idiopathic pulmonary fibrosis, cystic fibrosis, thermal injury, stroke, experimental allergic encephalomyelitis, multiple organ injury syndrome secondary to trauma, neutrophilic dermatosis (Sweet's disease), glomerulonephritis, ulcerative colitis, Crohn's disease, necrotizing enterocolitis, cytokine-induced toxicity, gingivitis, periodontitis, hemolytic uremic syndrome, psoriasis, systemic lupus erythematosus, autoimmune thyroiditis, multiple sclerosis, rheumatoid arthritis, Grave's disease, immunological-mediated side effects of treatment associated with hemodialysis or leukapheresis, granulocyte transfusion associated syndrome, deep vein thrombosis, post-thrombotic syndrome, unstable angina, transient ischemic attacks, peripheral vascular disease (e.g., peripheral artery disease), metastasis associated with cancer, sickle syndromes (including but not limited to sickle cell anemia), organ rejection (graft vs. host), or congestive heart failure.

In some embodiments, the disease, disorder, condition, or undesired process can be an undesired infection process mediated by a bacteria, a virus, or a parasite, for example gingivitis, periodontitis, hemolytic uremic syndrome, or granulocyte transfusion associated syndrome.

In some embodiments, the disease, disorder, condition, or undesired process can be metastasis associated with cancer. In further embodiments, the disease, disorder, condition, or undesired process can be a disease or disorder associated with an undesired immunological process, for example psoriasis, systemic lupus erythematosus, autoimmune thyroiditis, multiple sclerosis, rheumatoid arthritis, Grave's disease, and immunological-mediated side effects of treatment associated with hemodialysis or leukapheresis. In certain embodiments, the disease, disorder, condition, or undesired process can be a condition associated with an undesired thrombotic process, for example, deep vein thrombosis, unstable angina, transient ischemic attacks, peripheral vascular disease, post-thrombotic syndrome, venous thromboembolism, or congestive heart failure.

In some embodiments, the present teachings provide methods of ameliorating an undesired immunological process in a transplanted organ (e.g., renal transplant that include administering to the organ a compound of the present teachings or its pharmaceutically acceptable salt, hydrate, or ester. In some embodiments, the present teachings provide methods of treating, or ameliorating a symptom of a sickle syndrome, for example, sickle cell anemia, that include administering a compound of the present teachings to a patient in need thereof. In some embodiments, the methods can include identifying a human, mammal or animal that has a biomarker for a disease or disorder involving selectin-mediated intracellular adhesion, and administering to the human, mammal or animal a therapeutically effective amount of a compound described herein. In some embodiments, the biomarker can be one or more of soluble P-selectin, CD40, CD 40 ligand, MAC-1, TGF beta, ICAM, VCAM, IL-1. IL-6, IL-8, Eotaxin, RANTES, MCP-1, P1GF, CRP, SAA, and platelet monocyte aggregates.

Compounds of the present teachings can be prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

Compounds of the present teachings can by synthesized generally according to Schemes 1-6.

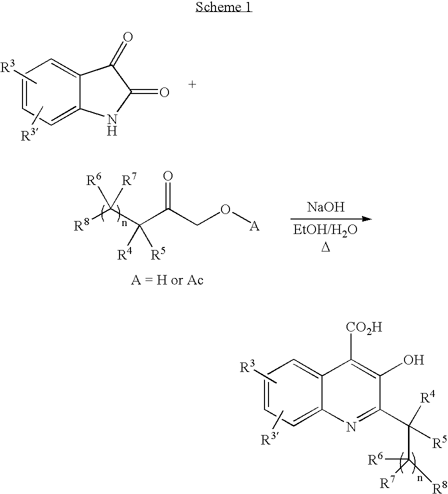

Compounds of the present teachings can be prepared by reacting an optionally substituted indoline-2,3-dione with an optionally substituted 2-oxo-propyl acetate or corresponding alcohol in the presence of a base, e.g. NaOH, as shown above in Scheme 1, wherein n, R³, R^{3′}, R⁴, R⁵, R⁶, R⁷, and R⁸ are as defined herein.

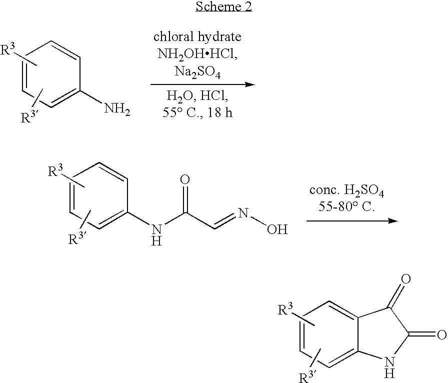

The substituted indoline-2,3-dione can be prepared from an appropriately substituted aniline as shown above in Scheme 2, wherein R³ and R^{3′} are as defined herein.

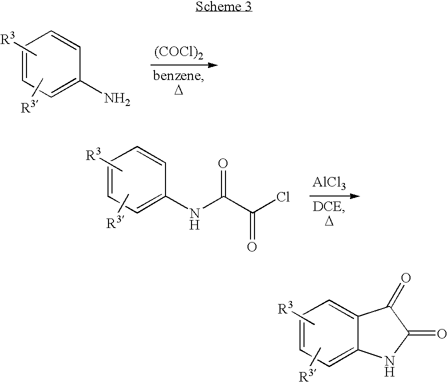

Alternatively, the substituted indoline-2,3-dione can be prepared from an appropriately substituted aniline as shown above in Scheme 3, wherein R³ and R^{3′} are as defined herein.

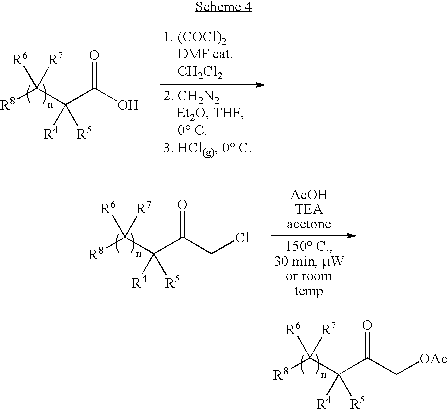

The substituted 2-oxo-propyl acetate can be prepared from an appropriately substituted carboxylic acid, as shown above in Scheme 4, wherein n, R⁴, R⁵, R⁶, R⁷, and R⁸ are as defined herein.

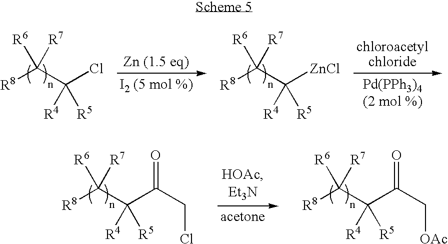

Alternatively, the substituted 2-oxo-propyl acetate can be prepared from an appropriately substituted halide, as shown above in Scheme 5, wherein n, R⁴, R⁵, R⁶, R⁷, and R⁸ are as defined herein.

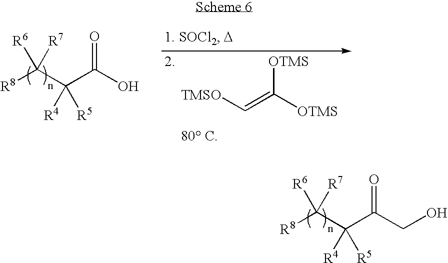

Alternatively, the corresponding alcohol of the substituted 2-oxo-propyl acetate can be prepared from the appropriately substituted carboxylic acid as shown above in Scheme 6, wherein n, R⁴, R⁵, R⁶, R⁷, and R⁸ are as defined herein.

### EXAMPLES

The following non-limiting examples are presented merely to illustrate the present teachings. A skilled person in the art will understand that there are numerous equivalents and variations that are not exemplified but still form part of the present teachings.

### Preparation of Intermediates

### Preparation of intermediate 1: 1-chloro-3-methyl-3-phenylbutan-2-one

To a 250 mL round-bottom flask under a nitrogen atmosphere was added 2-methyl-2-phenylpropanoic acid (5.0 g, 30.9 mmol, 1.0 eq.) and 100 mL of methylene chloride. To the resulting stirred solution was added oxalyl chloride (3.2 mL, 37.04 mmol, 1.2 eq.) and 3 drops of dimethylformamide (DMF). The mixture was stirred at room temperature until all gas evolution ceased. All volatile materials were removed in vacuo to give an oily solid. This material was redissolved into 50 mL of anhydrous tetrahydrofuran (THF) and added dropwise to 100 mL of an ethereal solution of diazomethane at 0° C. The resulting solution was allowed to warm slowly to room temperature and stirred for an additional 12 hours. The solution was cooled to 0° C. and hydrogen chloride (HCl) gas was bubbled through the solution for 5 minutes. Crushed ice was added to the mixture and stirring was continued for 15 minutes. The layers were separated and the aqueous layer was extracted with two 50 mL-portions of diethyl ether. The combined organic layers were washed with three 100 mL-portions of saturated sodium bicarbonate solution, three 100 mL-portions of water, and 100 mL of saturated sodium chloride solution. The solution was dried over magnesium sulfate, filtered, and the solvent was removed in vacuo to give intermediate 1 as a colorless oil (5.73 g, 94% yield). ¹H NMR (400 MHz, CDCl₃) δ 1.55 (s, 6H), 4.03 (s, 2H), 6.57-7.64 (m, 5H).

### Preparation of intermediate 2: 3-methyl-2-oxo-3-phenylbutyl acetate

To a 20 mL microwave-reaction vial was added intermediate 1 (1-chloro-3-methyl-3-phenylbutan-2-one, 5.73 g, 29.16 mmol, 1.0 eq.) and 15 mL of acetone. To the resulting solution was added acetic acid (2.2 mL, 37.9 mmol, 1.3 eq.) and triethylamine (5.3 mL, 37.9 mmol, 1.3 eq.). The vial was sealed and heated to 150° C. in a microwave reactor for 30 minutes. The resulting suspension was poured into 200 mL of water and extracted with three 100 mL-portions of ethyl acetate. The combined organic layers were washed with three 250 mL-portions of water and 250 mL of saturated sodium chloride solution. The organic layer was dried over magnesium sulfate, filtered, and the solvent was removed to give a brown oil. This was purified by silica gel chromatography (Biotage Flash 40, 0-10% ethyl acetate/hexanes) to give intermediate 2 as a white solid (4.75 g, 74% yield). ¹H NMR (400 MHz, CDCl₃) δ 1.55 (s, 6H), 2.10 (s, 3H), 4.56 (s, 2H), 6.58-7.98 (m, 5H).

### Preparation of intermediate 3: 6,7,8,9-tetrahydro-1H-benzo[g]indole-2,3-dione

The isatin synthesis described by Yang et al. (see J. Am. Chem. Soc., 1996, 118: 9557) was used. Chloral hydrate (3.28 g, 19.8 mmol), hydroxylamine hydrochloride (4.13 g, 59.4 mmol), and sodium sulfate (23 g, 165 mmol) were placed in a 500 mL round-bottom flask, and 120 mL of water was added. The suspension was heated to 55° C. under a N₂ balloon until all the solids were dissolved, and an emulsion of 5,6,7,8-tetrahydro-naphthalen-1-ylamine (Aldrich, 2.43 g, 16.5 mmol) in 2 M aqueous hydrochloric acid was added. Heating was continued overnight. After 18 hours, the reaction mixture was cooled to room temperature. The brown lumpy precipitate was collected by filtration, washed with water, and dried overnight to give isonitrosoacetanilide (3.4 g). The isonitrosoacetanilide (3.4 g) was added in small portions, with stirring, to 12.4 mL of concentrated sulfuric acid in a round-bottom flask at 65° C. After all the isonitrosoacetanilide had been added, the purplish-black solution was allowed to stir at 85° C. for 10 minutes, and was poured onto crushed ice in a beaker. Additional ice was added until the outside of the beaker felt cold to touch. The orange-brown precipitate was collected by filtration and dried overnight to yield isatin 3, which was purified by extraction. Intermediate 3 (5.7 g) was extracted with three 400 mL-portions of hot ethyl acetate and the insoluble solid was discarded. Evaporation of ethyl acetate gave 3.83 g of pure material. ¹H NMR (400 MHz, dimethylsulfoxide-d₆ (“DMSO-d₆”)) δ 1.74 (m, 4H), 2.50 (m, 2H), 2.74 (t, J=5.81 Hz, 2H), 6.79 (d, J=7.83 Hz, 1H), 7.23 (d, J=7.83 Hz, 1H), 10.95 (s, 1H).

### Preparation of intermediate 4: 6,7-dimethyl-1H-indole-2,3-dione

The isatin synthesis described by Rewcastle et al. (see J. Med. Chem., 1991, 34: 217) was used. Chloral hydrate (45 g, 0.27 mol), hydroxylamine hydrochloride (205 g, 1.25 mol), and sodium sulfate (226.5 g, 1.6 mol) were placed in a 2 L round-bottom flask, and 750 mL of water was added. To this suspension was added 2,3-dimethyl aniline (29.05 g, 0.24 mol) in 250 mL of water containing concentrated hydrochloric acid (HCl, 25 mL). The suspension was heated at 45° C. under N₂ for 90 minutes, then to 52° C. over 45 minutes, and at 75° C. for 60 minutes. The reaction mixture was cooled to room temperature. The precipitate was collected by filtration, washed with water and petroleum ether, and dried overnight in a vacuum desiccators to give crude N-(2,3-dimethyl-phenyl)-2-hydroxyimino-acetamide (40.1 g, 87%).

N-(2,3-Dimethyl-phenyl)-2-hydroxyimino-acetamide (20 g, 0.1 mol) was added in small portions, with stirring, to 80 mL of CH₃SO₃H at 70° C.-80° C. in one hour. After the addition was complete it was left at the same temperature for 15 minutes and was poured onto crushed ice in a beaker. Additional ice was added until the outside of the beaker felt cold to touch. The precipitate was collected and dissolved in 1N aqueous NaOH. Neutralization with acetic acid precipitated impurities which were removed by filtration, and acidification (HCl) of the filtrate gave intermediate 4 as a solid (12.8 g, 70% yield). ¹H NMR (400 MHz, DMSO-d₆) δ 2.09 (s, 3H), 2.27 (s, 3H), 6.89 (d, J=7.58 Hz, 1H), 7.25 (d, J=7.58 Hz, 1H), 11.02 (s, 1H).

### Preparation of intermediate 5: 7-isopropylindoline-2,3-dione

Intermediate 5 was prepared as a brown powder (46% yield) following the procedure used for intermediate 3. ¹H NMR (400 MHz, DMSO-d₆) δ 1.18 (d, J=6.8 Hz, 6H), 3.04 (sep, 1H), 7.06 (t, J=7.7 Hz, 1H), 7.35 (d, J=7.3 Hz, 1H), 7.54 (d, J=7.3 Hz, 1H), 11.09 (s, 1H). MS (electrospray) 188 (M-H)⁻.

### Preparation of intermediate 7: 2-chloro-1-(1-phenylcyclopropyl)ethanone

To a 250 mL round-bottom flask under a nitrogen atmosphere was added 1-phenylcyclopropanecarboxylic acid (5.0 g, 30.9 mmol, 1.0 eq.) and 100 mL of methylene chloride. To the resulting stirred solution was added oxalyl chloride (3.2 mL, 37.04 mmol, 1.2 eq.) and 3 drops of DMF. The mixture was stirred at room temperature until all gas evolution ceased. All volatile materials were removed in vacuo to give an oily solid. This material was redissolved into 50 mL of anhydrous THF and added dropwise to 100 mL of an ethereal solution of diazomethane cooled to 0° C. The resulting solution was allowed to warm slowly to room temperature and stirred for 12 hours. The solution was cooled once again to 0° C., and HCl gas was bubbled through the solution for 5 minutes. Crushed ice was added to the mixture and stirring was continued for 15 minutes. The layers were separated, and the aqueous layer was extracted with two 50 mL-portions of diethyl ether. The combined organic layers were washed with three 100 mL-portions of saturated sodium bicarbonate solution, three 100 mL-portions of water, and 100 mL of saturated sodium chloride solution. The solution was dried over magnesium sulfate, filtered, and the solvent was removed in vacuo to give intermediate 7 as a colorless oil (3.71 g, 61% yield). ¹H NMR (400 MHz, CDCl₃) δ 1.28 (q, J=3.79 Hz, 2H), 1.73 (q, J=3.37 Hz, 2H), 4.11 (s, 2H), 6.58-7.80 (m, 5H).

### Preparation of Intermediate 8: 2-oxo-2-(1-phenylcyclopropyl)ethyl acetate

To a 20 mL microwave-reaction vial was added intermediate 7 (2-chloro-1-(1-phenylcyclopropyl)ethanone, 3.71 g, 19.07 mmol, 1.0 eq.) and 15 mL of acetone. To the resulting solution was added acetic acid (1.41 mL, 24.8 mmol, 1.3 eq.) and triethylamine (3.5 mL, 24.8 mmol, 1.3 eq.). The vial was sealed and heated to 150° C. in a microwave reactor for 30 minutes. The resulting suspension was poured into 200 mL of water and extracted with three 100 mL-portions of ethyl acetate. The combined organic layers were washed with three 250 mL-portions of water and 250 mL of saturated sodium chloride solution. The organic layer was dried over magnesium sulfate, filtered, and the solvent was removed to give a brown oil, which was purified by silica gel chromatography (Biotage Flash 40, 0-10% ethyl acetate/hexanes) to give the desired product as a white solid (intermediate 8, 1.51 g, 36% yield). ¹H NMR (400 MHz, CDCl₃) δ 1.24 (q, J=3.54 Hz, 2H), 1.69 (q, J=3.54 Hz, 2H), 2.11 (s, 3H), 4.57 (s, 2H), 6.35-8.47 (m, 5H).

### Preparation of intermediate 9: 2-(hydroxyimino)-N-(2-iodophenyl)acetamide

The procedure described above for intermediate 3 was followed, reacting 2-iodoaniline (10 g, 46 mmol) with chloral hydrate (9.1 g, 55 mmol), hydroxylamine hydrochloride (11.4 g, 0.165 mol) and sodium sulfate (52 g, 0.366 mol) to give 2-(hydroxyimino)-N-(2-iodophenyl)acetamide as a beige solid (11.0 g, 83% yield). ¹H NMR (400 MHz, DMSO-d₆) δ 6.99 (t, J=7.7 Hz, 1H), 7.41 (t, 1H), 7.63 (s, 1H), 7.76 (dd, J=8.1, 1.3 Hz, 1H), 7.90 (dd, J=7.8, 1.3 Hz, 1H), 9.38 (s, 1H), 12.42 (s, 1H).

### Preparation of intermediate 10: 7-iodoindoline-2,3-dione

The procedure described above for intermediate 3 was followed, heating 2-(hydroxyimino)-N-(2-iodophenyl)acetamide (11.0 g, 38.0 mmol) in 30 mL of concentrated sulfuric acid to give a dark red powder (intermediate 10, 8.30 g, 80% yield). ¹H NMR (400 MHz, DMSO-d₆) δ 6.89 (t, J=7.7 Hz, 1H), 7.50 (d, J=7.3 Hz, 1H), 7.95 (d, J=6.8 Hz, 1H), 11.01 (s, 1H).

### Preparation of intermediate 11: 7-phenylindoline-2,3-dione

The procedure described by Lisowski et al. (see J. Org. Chem., 2000, 65: 4193) was followed. To a 1 L 3-neck round-bottom flask fitted with a reflux condenser were added 7-iodoindoline-2,3-dione (intermediate 10, 2.0 g, 7.33 mmol) and tetrakis[triphenylphosphine]palladium (0.424 g, 0.367 mmol), followed by 225 mL of 1,2-dimethoxyethane. The atmosphere in the reaction vessel was made inert by opening to vacuum, then to a positive pressure of nitrogen three times. Phenylboronic acid (Aldrich, 0.983 g, 8.06 mmol) and a solution of sodium bicarbonate (1.23 g, 14.7 mmol) in 225 mL of water were added, and the evacuation/nitrogen purge procedure was repeated one more time. The reaction mixture was heated at reflux temperature until thin layer chromatography (t.l.c.) (10% ethyl acetate in dichloromethane) showed complete disappearance of 7-iodoindoline-2,3-dione (1-2 hours). After cooling to room temperature, 1,2-dimethoxyethane was removed under reduced pressure. The residue was diluted with 1 M aqueous hydrochloric acid and extracted into ethyl acetate. The organic layer was washed with brine, dried over anhydrous magnesium sulfate, and filtered. Ethyl acetate was removed under reduced pressure to give crude 7-phenylindoline-2,3-dione.

This procedure was repeated eight additional times. The combined crude product was purified by flash chromatography over silica gel (1% ethyl acetate in dichloromethane) to give pure 7-phenylindoline-2,3-dione as orange needle-like crystals (10.94 g, 74% yield from 18 g of 7-iodoindoline-2,3-dione). ¹H NMR (400 MHz, DMSO-d₆) δ 7.18 (t, J=7.6 Hz, 1H), 7.48 (m, 6H), 7.59 (d, J=8.8 Hz, 1H), 10.91 (s, 1H).

### Preparation of intermediate 12: 2-(hydroxyimino)-N-(2-(trifluoromethoxy)phenyl)acetamide

Intermediate 12 was prepared following the procedure used for intermediate 3 (85% yield). ¹H NMR (400 MHz, DMSO-d₆) δ 7.31 (m, 1H), 7.42 (m, 2H), 7.75 (s, 1H), 7.97 (dd, J=7.8, 1.3 Hz, 1H), 9.71 (s, 1H), 12.39 (s, 1H).

### Preparation of intermediate 13: 7-(trifluoromethoxy)indoline-2,3-dione

The procedure described by Marvel et al. (see Org. Synth. Coll. Vol. I, 327) was followed. Intermediate 12 (11.9 g, 48.5 mmol) was added in small portions to 35 mL of concentrated sulfuric acid at 55° C. in a 250 mL Erlenmeyer flask. The temperature of the solution was maintained below 70° C. until all the acetamide had been added and it was increased to 80° C. for 10 minutes. The dark-colored solution was cooled to room temperature and poured onto 175 mL of crushed ice. After standing for 30 minutes, the precipitate was collected by filtration, washed three times with water, and dried under vacuum to yield indoline-2,3-dione of sufficient purity to be used in the next step (intermediate 13, 8.32 g, 70% yield). ¹H NMR (400 MHz, DMSO-d₆) δ 7.15 (t, J=7.8 Hz, 1H), 7.56 (d, J=7.3 Hz, 1H), 7.64 (d, J=8.3 Hz, 1H), 11.71 (s, 1H).

### Preparation of intermediate 14: N-(4-(1,1,1,3,3,3-hexafluoro-2-hydroxypropan-2-yl)phenyl)-2-(hydroxyimino)acetamide

To a 250 mL round-bottom flask was added 2-(4-aminophenyl)-1,1,1,3,3,3-hexafluoropropan-2-ol (2.0 g, 7.72 mmol, 1.0 eq.), chloral hydrate (1.53 g, 9.27 mmol, 1.2 eq.), hydroxylamine hydrochloride (1.9 g, 27.02 mmol, 3.5 eq.), sodium sulfate (10.97 g, 77.22 mmol, 10.0 eq.), 50 mL of water, and 12 mL of 1.2 N HCl. The resulting mixture was heated to 55° C. and allowed to stir for 15 hours. The resulting suspension was cooled to room temperature and the precipitated oxime intermediate 14 was obtained by filtration.

### Preparation of intermediate 15: 5-(1,1,1,3,3,3-hexafluoro-2-hydroxypropan-2-yl)indoline-2,3-dione

Crude intermediate 14 was added to 20 mL of concentrated sulfuric acid and heated to 80° C. for 10 minutes. 200 mL of crushed ice was added to this red/brown mixture and the resulting suspension was stirred for 30 minutes. Solids were collected by filtration and purified by silica gel chromatography (Biotage Flash 40, 25% ethyl acetate/hexane) to give the desired product as a yellow solid (intermediate 15, 1.25 g, 52% yield). ¹H NMR (400 MHz, DMSO-d₆) δ 7.08 (d, J=8.59 Hz, 1H), 7.52-7.70 (m, 2H), 7.77-7.93 (m, 1H), 8.93 (s, 1H).

### Preparation of intermediate 16: 7-(1,1,1,3,3,3-hexafluoro-2-hydroxypropan-2-yl)indoline-2,3-dione

To a 500 mL round-bottom flask was added 2-(2-aminophenyl)-1,1,1,3,3,3-hexafluoropropan-2-ol (9.0 g, 34.75 mmol, 1.0 eq.), chloral hydrate (6.9 g, 41.69 mmol, 1.2 eq.), hydroxylamine hydrochloride (8.45 g, 122.0 mmol, 3.5 eq.), sodium sulfate (49.34 g, 347.0 mmol, 10.0 eq.), 225 mL of water, and 55 mL of 1.2 N HCl. The resulting mixture was heated to 55° C. and allowed to stir for 15 hours. The resulting suspension was cooled to room temperature and the precipitated oxime intermediate was obtained by filtration. This white solid was added to 20 mL of concentrated sulfuric acid and heated to 80° C. for 10 minutes. Crushed ice (200 mL) was added to this red/brown mixture and the resulting suspension was stirred for 30 minutes. Solids were collected by filtration and purified by silica gel chromatography (Biotage Flash 40, 25% ethyl acetate/hexane) to give the desired product as a yellow solid (intermediate 16, 5.64 g, 52% yield). ¹H NMR (400 MHz, CDCl₃) δ 7.22 (dd, J=8.34, 7.33 Hz, 1H), 7.69 (d, J=9.35 Hz, 1H), 7.75 (dd, J=7.33, 1.26 Hz, 1H).

### Preparation of intermediate 17: 2-chloro-1-(1-(4-methoxyphenyl)cyclopropyl)ethanone

To a 25 mL round-bottom flask under a nitrogen atmosphere was added 1-(4-methoxyphenyl)cyclopropanecarboxylic acid (0.96 g, 5.0 mmol, 1.0 eq.) and 5 mL of methylene chloride. Oxalyl chloride (0.6 mL, 6.5 mmol, 1.3 eq.) and 1 drop of DMF were added, and the mixture was allowed to stir until all gas evolution ceased. All volatiles were removed in vacuo and the resulting residue was re-dissolved into 5 mL of THF. This solution was added dropwise to 20 mL of an ethereal solution of diazomethane cooled to 0° C. The resulting solution was allowed to warm slowly to room temperature and stir for 12 hours. The solution was cooled to 0° C. and HCl gas was bubbled through for 3 minutes. Crushed ice was added to the mixture and stirring was continued for 15 minutes. The layers were separated and the aqueous layer was extracted with two 50 mL-portions of diethyl ether. The combined organic layers were washed with three 100 mL-portions of saturated sodium bicarbonate solution, three 100 mL-portions of water, and 100 mL of saturated sodium chloride solution. The solution was dried over magnesium sulfate, filtered, and the solvent was removed in vacuo to give intermediate 17 as a colorless oil (0.327 g, 30% yield). ¹H NMR (400 MHz, CDCl₃) δ 1.20 (q, J=3.54 Hz, 2H), 1.66 (q, J=3.37 Hz, 2H), 3.82 (s, 3H), 4.32 (s, 2H), 6.89 (d, J=8.84 Hz, 2H), 7.34 (d, J=8.84 Hz, 2H).

### Preparation of intermediate 18: 2-(1-(4-methoxyphenyl)cyclopropyl)-2-oxoethyl acetate

To a 20 mL microwave-reaction vial was added intermediate 17 (2-chloro-1-(1-(4-methoxyphenyl)cyclopropyl)ethanone, 0.327 g, 1.48 mmol, 1.0 eq.) and 5 mL of acetone. To the resulting solution was added acetic acid (0.11 mL, 1.92 mmol, 1.3 eq.) and triethylamine (0.27 mL, 1.92 mmol, 1.3 eq.). The vial was sealed and heated at 150° C. in a microwave reactor for 30 minutes. The resulting suspension was poured into 50 mL of water and extracted with three 25 mL-portions of ethyl acetate. The combined organic layers were washed with three 75 mL-portions of water and 75 mL of saturated sodium chloride solution. The organic layer was dried over magnesium sulfate, filtered, and the solvent was removed to give a brown oil. This was purified by silica gel chromatography (Biotage Flash 40, 0-10% ethyl acetate/hexanes) to give the desired product as a white solid (intermediate 18, 0.144 g, 40% yield). ¹H NMR (400 MHz, CDCl₃) δ 1.20 (q, J=3.54 Hz, 2H), 1.66 (q, J=3.37 Hz, 2H), 2.11 (s, 3H), 3.82 (s, 3H), 4.58 (s, 2H), 6.89 (d, J=8.84 Hz, 2H), 7.34 (d, J=8.84 Hz, 2H).

### Preparation of intermediate 19: 1-(4-(trifluoromethyl)phenyl)cyclopropanecarbonitrile

This compound was prepared following the procedure described by Jonczyk et al. (see Org. Prep. Proc. Int, 1995, 27(3): 355-359). To a 25 mL round-bottom flask equipped with a condenser was added 2-(4-(trifluoromethyl)phenyl)acetonitrile (0.75 g, 4.05 mmol, 1.0 eq.), 1-bromo-2-chloroethane (0.50 mL, 6.08 mmol, 1.5 eq.), and triethylbenzyl ammonium chloride (0.018 g, 0.08 mmol, 0.02 eq.). The resulting mixture was heated to 50° C. and sodium hydroxide (0.97 g, 24.0 mmol, 6.0 eq. dissolved in 1.0 mL of water) was added dropwise. The mixture was allowed to stir at 50° C. for 16 hours. It was cooled to room temperature and poured into 50 mL of water. This suspension was extracted with three 25 mL-portions of methylene chloride and the combined organic layers washed with three 50 mL-portions of 1.2 N HCl aqueous solution, three 50 mL-portions of water, and 50 ml of saturated sodium chloride solution. The organic layer was dried over magnesium sulfate, filtered, and the solvent was removed in vacuo. The crude material was purified by silica gel chromatography (Biotage Flash 40, 10% ethyl acetate/hexanes) to give the desired product as a light yellow oil (intermediate 19, 0.74 g, 86% yield). ¹H NMR (400 MHz, CDCl₃) δ 1.41-1.53 (m, 2H), 1.78-1.87 (m, 2H), 7.40 (d, J=8.34 Hz, 2H), 7.62 (d, J=8.34 Hz, 2H).

### Preparation of intermediate 20: 1-(4-(trifluoromethyl)phenyl)cyclopropanecarboxylic acid

To a 50 mL round-bottom flask equipped with a condenser was added intermediate 19 (1-(4-(trifluoromethyl)phenyl)cyclopropanecarbonitrile, 0.55 g, 2.5 mmol, 1.0 eq.) and 20 mL of 4.0 N LiOH aqueous solution. This suspension was heated at reflux temperature and allowed to stir for 15 hours. The resulting mixture was cooled to room temperature and poured into 250 mL of 1.2 N HCl solution. This suspension was extracted with three 75 mL-portions of ethyl acetate and the combined organic layers were washed with three 200 mL-portions of water and 200 mL of saturated sodium chloride solution. The organic layer was dried over magnesium sulfate, filtered, and the solvent was removed in vacuo. The desired product was obtained as a white solid (intermediate 20, 0.564 g, 95% yield). ¹H NMR (400 MHz, CDCl₃) δ 1.29 (q, J=3.87 Hz, 2H), 1.72 (q, J=3.87 Hz, 2H), 7.46 (d, J=8.08 Hz, 2H), 7.57 (d, J=8.08 Hz, 2H).

### Preparation of intermediate 21: 2-hydroxy-1-(1-(4-(trifluoromethyl)phenyl)cyclopropyl)ethanone

To a 50 mL round-bottom flask equipped with a condenser was added intermediate 20 (1-(4-(trifluoromethyl)phenyl)cyclopropanecarboxylic acid, 0.270 g, 1.18 mmol, 1.0 eq.) and 25 mL of thionyl chloride. This mixture was heated at reflux temperature and allowed to stir for 4 hours. It was allowed to cool to room temperature and all volatiles were removed in vacuo. To the resulting yellow oil was added tris(trimethylsilyloxy)ethylene (0.757 g, 2.59 mmol, 2.2 eq.) and the mixture was heated to 80° C. and allowed to stir for 12 hours. To this mixture was added a solution of 15 mL of 1.2 N HCl solution, 10 mL of water, and 35 mL of dioxane. This mixture was heated at reflux temperature and allowed to stir for 1 hour. Upon cooling, the mixture was extracted with three 50 mL-portions of ethyl acetate and the combined organic layers were washed with three 100 mL-portions of saturated sodium bicarbonate solution, three 100 mL-portions of water, and 100 mL of saturated sodium chloride solution. The organic layer was dried over magnesium sulfate, filtered, and the solvent was removed in vacuo. The crude oil was purified by silica gel chromatography (Biotage Flash 40, 10-25% ethyl acetate/hexanes) to give the desired product as a colorless oil (intermediate 21, 0.149 g, 52% yield). ¹H NMR (400 MHz, CDCl₃) δ 1.32 (q, J=3.96 Hz, 2H), 1.79 (q, J=3.79 Hz, 2H), 4.05 (s, 2H), 7.51 (d, J=7.83 Hz, 2H), 7.64 (d, J=8.08 Hz, 2H).

### Preparation of intermediate 22: 1-(4-bromophenyl)cyclopropanecarbonitrile

Intermediate 22 was synthesized by the method used for intermediate 19, using as starting materials 2-(4-bromophenyl)acetonitrile (0.79 g, 4.05 mmol, 1.0 eq.), 1-bromo-2-chloroethane (0.50 mL, 6.08 mmol, 1.5 eq.), triethylbenzyl ammonium chloride (0.018 g, 0.08 mmol, 0.02 eq.), and sodium hydroxide (0.97 g, 24.0 mmol, 6.0 eq. dissolved into 1.0 mL of water). The desired product was obtained as a white solid (intermediate 22, 0.55 g, 61% yield). ¹H NMR (400 MHz, CDCl₃) δ 1.33-1.44 (m, 2H), 1.68-1.79 (m, 2H), 7.16 (d, J=8.59 Hz, 2H), 7.48 (d, J=8.84 Hz, 2H).

### Preparation of intermediate 23: 1-(4-bromophenyl)cyclopropanecarboxylic acid

Intermediate 23 was synthesized by the method used for intermediate 20, using as starting material 1-(4-bromophenyl)cyclopropanecarbonitrile (0.548 g, 2.5 mmol, 1.0 eq.). The desired product was obtained as a white solid (intermediate 23, 0.56 g, 95% yield). ¹H NMR (400 MHz, CDCl₃) δ 1.23 (q, J=3.96 Hz, 2H), 1.58-1.71 (m, 2H), 7.21 (d, J=8.34 Hz, 2H), 7.43 (d, J=8.34 Hz, 2H).

### Preparation of intermediate 24: 1-(1-(4-bromophenyl)cyclopropyl)-2-chloroethanone

To a 50 mL round-bottom flask equipped with a condenser was added intermediate 23 (1-(4-bromophenyl)cyclopropanecarboxylic acid, 0.255 g, 1.06 mmol, 1.0 eq.) and 25 mL of thionyl chloride. The resulting solution was heated at reflux temperature and allowed to stir for 4 hours. Upon cooling to room temperature, all of volatiles were removed in vacuo. The resulting brown oil was redissolved into 10 mL of THF and added dropwise to 100 mL of an ethereal diazomethane solution cooled to 0° C. This mixture was allowed to warm slowly to room temperature and stir for 12 hours. The solution was cooled to 0° C. and HCl gas was bubbled through for 3 minutes. Crushed ice was added to the mixture and stirring was continued for 15 minutes. The layers were separated and the aqueous layer was extracted with two 50 mL-portions of diethyl ether. The combined organic layers were washed with three 100 mL-portions of saturated sodium bicarbonate solution, three 100 mL-portions of water, and 100 mL of saturated sodium chloride solution. The solution was dried over magnesium sulfate, filtered, and the solvent was removed in vacuo to give intermediate 24 as a colorless oil (0.287 g, 100% yield). ¹H NMR (400 MHz, CDCl₃) δ 1.25 (q, J=3.96 Hz, 2H), 1.74 (q, J=3.62 Hz, 2H), 4.08 (s, 2H), 7.28 (d, J=8.59 Hz, 2H), 7.52 (d, J=8.34 Hz, 2H).

### Preparation of intermediate 25: 2-(1-(4-bromophenyl)cyclopropyl)-2-oxoethyl acetate

Intermediate 25 was synthesized by the method used for intermediate 18, using as starting materials intermediate 24 (1-(1-(4-bromophenyl)cyclopropyl)-2-chloroethanone, 0.287 g, 1.06 mmol, 1.0 eq.), acetic acid (0.08 mL, 1.4 mmol, 1.3 eq.), and triethylamine (0.3 mL, 1.3 mmol, 1.3 eq.). The desired product was obtained as a white solid (intermediate 25, 0.091 g, 30% yield). ¹H NMR (400 MHz, CDCl₃) δ 1.21 (q, J=3.87 Hz, 2H), 1.69 (q, J=3.79 Hz, 2H), 2.11 (s, 3H), 4.55 (s, 2H), 7.31 (d, J=8.59 Hz, 2H), 7.51 (d, J=8.59 Hz, 2H).

### Preparation of intermediate 26: 1-(3-chlorophenyl)cyclopropanecarbonitrile

Intermediate 26 was synthesized by the method used for intermediate 19, using as starting materials 2-(3-chlorophenyl)acetonitrile (1.0 g, 6.6 mmol, 1.0 eq.), 1-bromo-2-chloroethane (0.82 mL, 9.9 mmol, 1.5 eq.), and triethylbenzylammonium chloride (0.030 g, 0.13 mmol, 0.02 eq.). The desired product was obtained as a yellow oil (intermediate 26, 1.2 g, 100% yield). ¹H NMR (400 MHz, CDCl₃) δ 1.36-1.45 (m, 2H), 1.69-1.81 (m, 2H), 6.38-7.94 (m, 5H).

### Preparation of intermediate 27: 1-(3-chlorophenyl)cyclopropanecarboxylic acid

Intermediate 27 was synthesized by the method used for intermediate 20, using as starting materials intermediate 26 (1-(3-chlorophenyl)cyclopropanecarbonitrile, 1.2 g, 6.6 mmol, 1.0 eq.), and was obtained as a white solid (0.81 g, 62% yield). This material was converted to intermediate 28 without further analysis.

### Preparation of intermediate 28: 1-(1-(3-chlorophenyl)cyclopropyl)-2-hydroxyethanone

Intermediate 28 was synthesized by the method used for intermediate 21, using as starting materials intermediate 27 (1-(3-chlorophenyl)cyclopropanecarboxylic acid, 0.81 g, 4.08 mmol, 1.0 eq.), thionyl chloride (20 mL, large excess), and tris(trimethylsilyloxy)ethylene (2.64 g, 9.0 mmol, 2.2 eq.), and was obtained as a colorless oil (0.396 g, 46% yield). ¹H NMR (400 MHz, CDCl₃) δ 1.30 (q, J=3.79 Hz, 2H), 1.74 (q, J=3.62 Hz, 2H), 3.16 (t, J=4.67 Hz, 1H), 4.08 (d, J=4.80 Hz, 2H), 5.97-8.14 (m, 4H).

### Preparation of intermediate 29: 1-(2-chlorophenyl)cyclopropanecarbonitrile

Intermediate 29 was synthesized by the method used for intermediate 19, using as starting materials 2-(2-chlorophenyl)acetonitrile (1.0 g, 6.6 mmol, 1.0 eq.), 1-bromo-2-chloroethane (0.82 mL, 9.9 mmol, 1.5 eq.), and triethylbenzylammonium chloride (0.030 g, 0.13 mmol, 0.02 eq.), and was obtained as a yellow oil (1.2 g, 100% yield). ¹H NMR (400 MHz, CDCl₃) δ 1.31-1.38 (m, 2H), 1.71-1.79 (m, 2H), 6.55-7.78 (m, 4H).

### Preparation of intermediate 30: 1-(2-chlorophenyl)cyclopropanecarboxylic acid

Intermediate 30 was synthesized by the method used for intermediate 20, using as starting materials intermediate 26 (1-(3-chlorophenyl)cyclopropanecarbonitrile, 1.2 g, 6.6 mmol, 1.0 eq.), and was obtained as a white solid (1.045 g, 90% yield). This material was converted to intermediate 31 without further analysis.

### Preparation of intermediate 31: 2-chloro-1-(1-(2-chlorophenyl)cyclopropyl)ethanone

Intermediate 31 was synthesized by the method used for intermediate 24, using as starting materials intermediate 30 (1-(2-chlorophenyl)cyclopropanecarboxylic acid, 1.05 g, 6.6 mmol, 1.0 eq.), thionyl chloride (20 mL, excess), and diazomethane (100 mL of ethereal solution, excess), and was obtained as a yellow oil (1.03 g, 68% yield). ¹H NMR (400 MHz, CDCl₃) δ 1.30 (d, J=3.79 Hz, 2H), 1.86 (d, J=3.79 Hz, 2H), 4.11 (s, 2H), 6.78-7.81 (m, 4H).

### Preparation of intermediate 32: 2-(1-(2-chlorophenyl)cyclopropyl)-2-oxoethyl acetate

Intermediate 32 was synthesized by the method used for intermediate 25, using as starting materials intermediate 31 (2-chloro-1-(1-(2-chlorophenyl)cyclopropyl)ethanone, 1.03 g, 4.5 mmol, 1.0 eq.), acetic acid (0.34 mL, 5.85 mmol, 1.3 eq.), and triethylamine (0.81 mL, 5.85 mmol, 1.3 eq.), and was obtained as a tan solid (0.36 g, 32% yield). ¹H NMR (400 MHz, CDCl₃) δ 1.26 (d, J=3.79 Hz, 2H), 1.82 (d, J=3.79 Hz, 2H), 2.11 (s, 3H), 4.59 (s, 2H), 7.28-7.35 (m, 2H), 7.39-7.53 (m, 2H).

### Preparation of intermediate 33: 1-(4-(trifluoromethoxy)phenyl)cyclopropane carbonitrile

Intermediate 33 was synthesized by the method used for intermediate 19, using as starting materials 2-(4-(trifluoromethoxy)phenyl)acetonitrile (1.0 g, 4.97 mmol, 1.0 eq.), 1-bromo-2-chloroethane (0.62 mL, 7.5 mmol, 1.5 eq.), and triethylbenzylammonium chloride (0.023 g, 0.10 mmol, 0.02 eq.), and was obtained as a yellow oil. ¹H NMR (400 MHz, CDCl₃) δ 1.22-1.49 (m, 2H), 1.66-1.85 (m, 2H), 7.20 (d, J=7.83 Hz, 2H), 7.33 (d, J=8.84 Hz, 2H).

### Preparation of intermediate 34: 1-(4-(trifluoromethoxy)phenyl)cyclopropane carboxylic acid

Intermediate 34 was synthesized by the method used for intermediate 20, using as starting materials intermediate 26 (1-(3-chlorophenyl)cyclopropanecarbonitrile, 1.14 g, 4.97 mmol, 1.0 eq.), and was obtained as a white solid (0.895 g, 73% yield over 2 steps). ¹H NMR (400 MHz, CDCl₃) δ 1.20-1.30 (m, 2H), 1.55-1.77 (m, 2H), 7.14 (d, J=8.08 Hz, 2H), 7.36 (d, J=8.59 Hz, 2H).

### Preparation of intermediate 35: 2-hydroxy-1-(1-(4-(trifluoromethoxy)phenyl)cyclopropyl)ethanone

Intermediate 35 was synthesized by the method used for intermediate 21, using as starting materials intermediate 34 (1-(4-(trifluoromethoxy)phenyl)cyclopropanecarboxylic acid, 0.895 g, 3.64 mmol, 1.0 eq.), thionyl chloride (20 mL, large excess), and tris(trimethylsilyloxy)ethylene (2.34 g, 8.0 mmol, 2.2 eq.), and was obtained as a colorless oil (0.527 g, 56% yield). ¹H NMR (400 MHz, CDCl₃) δ 1.30 (q, J=3.71 Hz, 2H), 1.76 (q, J=3.62 Hz, 2H), 3.16 (t, J=4.29 Hz, 1H), 4.05 (d, J=4.29 Hz, 2H), 7.22 (d, J=7.83 Hz, 2H), 7.41 (d, J=8.84 Hz, 2H).

### Preparation of intermediate 36: 1-(3-(trifluoromethyl)phenyl)cyclopropane carbonitrile

Intermediate 36 was synthesized by the method used for intermediate 19, using as starting materials 2-(3-(trifluoromethyl)phenyl)acetonitrile (1.0 g, 5.4 mmol, 1.0 eq.), 1-bromo-2-chloroethane (0.67 mL, 8.1 mmol, 1.5 eq.), and triethylbenzylammonium chloride (0.024 g, 0.11 mmol, 0.02 eq.), and was obtained as a yellow oil. ¹H NMR (400 MHz, CDCl₃) δ 1.43-1.49 (m, 2H), 1.77-1.86 (m, 2H), 7.40-7.62 (m, 4H).

### Preparation of intermediate 37: 1-(3-(trifluoromethyl)phenyl)cyclopropanecarboxylic acid

Intermediate 37 was synthesized by the method used for intermediate 20, using as starting materials intermediate 36 (1-(3-(trifluoromethyl)phenyl)cyclopropanecarbonitrile, 1.15 g, 5.4 mmol, 1.0 eq.), and was obtained as a white solid (1.03 g, 82% yield over 2 steps). ¹H NMR (400 MHz, CDCl₃) δ 1.26-1.32 (m, 2H), 1.64-1.77 (m, 2H), 7.42 (t, J=7.71 Hz, 1H), 7.49-7.57 (m, 2H), 7.59 (s, 1H).

### Preparation of intermediate 38: 2-hydroxy-1-(1-(3-(trifluoromethyl)phenyl)cyclopropyl)ethanone

Intermediate 38 was synthesized by the method used for intermediate 21, using as starting materials intermediate 37 (1-(3-(trifluoromethyl)phenyl)cyclopropanecarboxylic acid, 1.03 g, 4.5 mmol, 1.0 eq.), thionyl chloride (20 mL, large excess), and tris(trimethylsilyloxy)ethylene (2.88 g, 9.85 mmol, 2.2 eq.), and was obtained as a colorless oil (0.687 g, 62% yield). ¹H NMR (400 MHz, CDCl₃) δ 1.34 (q, J=3.87 Hz, 2H), 1.80 (q, J=3.62 Hz, 2H), 3.17 (t, J=4.80 Hz, 1H), 4.04 (d, J=4.80 Hz, 2H), 7.40-7.70 (m, 4H).

### Preparation of intermediate 39: 1-chloro-3-phenylbutan-2-one

Intermediate 39 was synthesized by the method used for intermediate 1, using as starting materials 2-phenylpropanoic acid (3.29 g, 21.91 mmol, 1.0 eq.) and oxalyl chloride (2.3 mL, 26.3 mmol, 1.2 eq.), and was obtained as a colorless oil (3.80 g, 95% yield). This material was converted to intermediate 40 without further analysis.

### Preparation of intermediate 40: 2-oxo-3-phenylbutyl acetate

Intermediate 40 was synthesized by the method used for intermediate 2, using as starting materials intermediate 39 (1-chloro-3-phenylbutan-2-one, 3.80 g, 20.8 mmol, 1.0 eq.), acetic acid (1.6 mL, 27.0 mmol, 1.3 eq.), and triethylamine (3.8 mL, 27.0 mmol, 1.3 eq.), and was obtained as a waxy tan solid (3.4 g, 79% yield). ¹H NMR (400 MHz, CDCl₃) δ 1.44 (d, J=7.07 Hz, 3H), 2.12 (s, 3H), 3.81 (q, J=7.07 Hz, 1H), 4.52 (d, J=16.67 Hz, 1H), 4.69 (d, J=16.67 Hz, 1H), 7.17-7.41 (m, 5H).

Alternatively, intermediate 40 can be synthesized by the following procedure. In a flame-dried 100 mL 2-necked round-bottom flask, under an inert atmosphere, was placed 0.5 M solution of (1-phenylethyl)zinc(II) bromide in THF (25 mL, 12.5 mmol). The reaction mixture was cooled to 0° C., and Pd(PPh₃)₄ (0.288 g, 0.25 mmol) was added, followed by dropwise addition, via syringe, of chloroacetyl chloride (1.5 mL, 18.8 mmol) in 6 mL of THF. The brown suspension was allowed to stir overnight at room temperature. To work up the reaction, 12 mL of 1 M hydrochloric acid was added and the mixture extracted with four 12 mL-portions of ethyl acetate. The combined organic layers were washed with brine, dried over anhydrous MgSO₄, filtered, and concentrated. This crude material was converted to intermediate 40 following the procedure for intermediate 21.

### Preparation of intermediate 41: 2-chloro-1-(1-(4-chlorophenyl)cyclobutyl)ethanone

Intermediate 41 was synthesized by the method used for intermediate 1, using as starting materials 1-(4-chlorophenyl)cyclobutanecarboxylic acid (2.0 g, 9.50 mmol, 1.0 eq.) and oxalyl chloride (1.0 mL, 11.40 mmol, 1.2 eq.), and was obtained as a colorless oil (2.30 g, 100% yield). ¹H NMR (400 MHz, CDCl₃) δ 1.70-2.09 (m, 2H), 2.34-2.51 (m, 2H), 2.66-3.00 (m, 2H), 4.00 (s, 2H), 7.18 (d, J=8.84 Hz, 2H), 7.36 (d, J=8.84 Hz, 2H).

### Preparation of intermediate 42: 2-(1-(4-chlorophenyl)cyclobutyl)-2-oxoethyl acetate

Intermediate 42 was synthesized by the method used for intermediate 2, using as starting materials intermediate 41 (2-chloro-1-(1-(4-chlorophenyl)cyclobutyl)ethanone, 2.3 g, 9.5 mmol, 1.0 eq.), acetic acid (0.71 mL, 12.35 mmol, 1.3 eq.), and triethylamine (1.72 mL, 12.35 mmol, 1.3 eq.), and was obtained as a waxy tan solid (1.69 g, 67% yield). ¹H NMR (400 MHz, CDCl₃) δ 1.74-2.04 (m, 2H), 2.12 (s, 3H), 2.33-2.49 (m, 2H), 2.68-2.97 (m, 2H), 4.47 (s, 2H), 7.18 (d, J=8.34 Hz, 2H), 7.35 (d, J=8.34 Hz, 2H).

### Preparation of intermediate 43: 1-(thiophen-3-yl)cyclopropanecarbonitrile

Intermediate 43 was synthesized by the method used for intermediate 19, using as starting materials 2-(thiophen-3-yl)acetonitrile (1.0 g, 8.12 mmol, 1.0 eq.), 1-bromo-2-chloroethane (1.0 mL, 12.18 mmol, 1.5 eq.), and triethylbenzylammonium chloride (0.037 g, 0.16 mmol, 0.02 eq.), and was obtained as a colorless oil (0.34 g, 28% yield). ¹H NMR (400 MHz, CDCl₃) δ 1.27-1.41 (m, 2H), 1.62-1.74 (m, 2H), 6.91 (dd, J=5.05, 1.26 Hz, 1H), 7.18 (dd, J=3.03, 1.52 Hz, 1H), 7.31 (dd, J=5.05, 3.03 Hz, 1H).

### Preparation of intermediate 44: 1-(thiophen-3-yl)cyclopropanecarboxylic acid

Intermediate 44 was synthesized by the method used for intermediate 20, using as starting materials intermediate 43 (1-(thiophen-3-yl)cyclopropanecarbonitrile, 0.34 g, 2.27 mmol, 1.0 eq.), and was obtained as a white solid (0.356 g, 93% yield). ¹H NMR (400 MHz, CDCl₃) δ 1.17-1.31 (m, 2H), 1.62-1.70 (m, 2H), 7.09 (dd, J=5.05, 1.01 Hz, 1H), 7.16 (dd, J=3.03, 1.26 Hz, 1H), 7.21-7.29 (m, 1H).

### Preparation of intermediate 45: 2-hydroxy-1-(1-(thiophen-3-yl)cyclopropyl)ethanone

Intermediate 45 was synthesized by the method used for intermediate 21, using as starting materials intermediate 44 (1-(thiophen-3-yl)cyclopropanecarboxylic acid, 0.356 g, 2.12 mmol, 1.0 eq.) and tris(trimethylsilyloxy)ethylene (1.54 mL, 4.66 mmol, 2.2 eq.), and was obtained as a colorless oil (0.062 g, 16% yield). ¹H NMR (400 MHz, CDCl₃) δ 1.29 (q, J=3.54 Hz, 2H), 1.69 (q, J=3.54 Hz, 2H), 3.15 (t, J=4.80 Hz, 1H), 4.15 (d, J=4.80 Hz, 2H), 7.05 (dd, J=5.05, 1.26 Hz, 1H), 7.23 (dd, J=3.03, 1.52 Hz, 1H), 7.34 (dd, J=4.93, 2.91 Hz, 1H).

### Preparation of intermediate 46: 1-(thiophen-2-yl)cyclopropanecarbonitrile

Intermediate 46 was synthesized by the method used for intermediate 19, using as starting materials 2-(thiophen-2-yl)acetonitrile (1.0 g, 8.12 mmol, 1.0 eq.), 1-bromo-2-chloroethane (1.0 mL, 12.18 mmol, 1.5 eq.), and triethylbenzylammonium chloride (0.037 g, 0.16 mmol, 0.02 eq.). The desired product was obtained as a colorless oil (intermediate 46, 1.20 g, 100% yield). ¹H NMR (400 MHz, CDCl₃) δ 1.37-1.49 (m, 2H), 1.67-1.82 (m, 2H), 6.94 (dd, J=5.18, 3.66 Hz, 1H), 7.06 (dd, J=3.54, 1.26 Hz, 1H), 7.19 (dd, J=5.05, 1.26 Hz, 1H).

### Preparation of intermediate 47: 1-(thiophen-2-yl)cyclopropanecarboxylic acid

Intermediate 47 was synthesized by the method used for intermediate 20, using as starting materials intermediate 46 (1-(thiophen-2-yl)cyclopropanecarbonitrile, 1.20 g, 8.12 mmol, 1.0 eq.). The desired product was obtained as a white solid (intermediate 47, 1.16 g, 85% yield). ¹H NMR (400 MHz, CDCl₃) δ 1.40 (q, J=3.96 Hz, 2H), 1.77 (q, J=3.87 Hz, 2H), 6.90-6.93 (m, 1H), 6.96 (dd, J=3.54, 1.26 Hz, 1H), 7.20 (dd, J=5.05, 1.26 Hz, 1H).

### Preparation of intermediate 48: 2-hydroxy-1-(1-(thiophen-2-yl)cyclopropyl)ethanone

Intermediate 48 was synthesized by the method used for intermediate 21, using as starting materials intermediate 47 (1-(thiophen-2-yl)cyclopropanecarboxylic acid, 1.16 g, 6.9 mmol, 1.0 eq.) and tris(trimethylsilyloxy)ethylene (5.0 mL, 15.2 mmol, 2.2 eq.). The desired product was obtained as a colorless oil (intermediate 48, 0.387 g, 31% yield). ¹H NMR (400 MHz, CDCl₃) δ 1.43 (q, J=3.79 Hz, 2H), 1.80 (q, J=3.54 Hz, 2H), 3.12 (t, J=4.80 Hz, 1H), 4.28 (d, J=4.80 Hz, 2H), 6.99 (dd, J=5.31, 3.54 Hz, 1H), 7.04 (dd, J=3.54, 1.26 Hz, 1H), 7.28 (dd, J=5.31, 1.26 Hz, 1H).

### Preparation of intermediate 49: 1-(4-fluorophenyl)cyclopropanecarbonitrile

Intermediate 49 was synthesized by the method used for intermediate 19 with the modification that the reaction mixture was stirred for 5 days at 50° C., using as starting materials 2-(4-fluorophenyl)acetonitrile (2.0 g, 14.8 mmol, 1.0 eq.), 1-bromo-2-chloroethane (2.45 mL, 29.6 mmol, 2.0 eq.), and triethylbenzylammonium chloride (0.067 g, 0.3 mmol, 0.02 eq.). The desired product was obtained as a colorless oil (intermediate 49, 1.52 g, 63% yield). ¹H NMR (400 MHz, CDCl₃) δ 1.27-1.42 (m, 2H), 1.56-1.80 (m, 2H), 6.94-7.10 (m, 2H), 7.19-7.40 (m, 2H).

### Preparation of intermediate 50: 1-(4-fluorophenyl)cyclopropanecarboxylic acid

Intermediate 50 was synthesized by the method used for intermediate 20, using as starting materials intermediate 49 (1-(4-fluorophenyl)cyclopropanecarbonitrile, 1.52 g, 9.32 mmol, 1.0 eq.), and was obtained as a white solid (1.64 g, 98% yield). ¹H NMR (400 MHz, CDCl₃) δ 1.23 (q, J=4.04 Hz, 2H), 1.66 (q, J=4.04 Hz, 2H), 6.91-7.04 (m, 2H) 7.21-7.38 (m, 2H).

### Preparation of intermediate 51: 1-(1-(4-fluorophenyl)cyclopropyl)-2-hydroxyethanone

Intermediate 51 was synthesized by the method used for intermediate 21, using as starting materials intermediate 50 (1-(4-fluorophenyl)cyclopropanecarboxylic acid, 1.64 g, 9.11 mmol, 1.0 eq.) and tris(trimethylsilyloxy)ethylene (6.6 mL, 20.0 mmol, 2.2 eq.), and was obtained as a colorless oil (0.824 g, 47% yield). ¹H NMR (400 MHz, CDCl₃) δ 1.28 (q, J=3.79 Hz, 2H), 1.74 (q, J=3.71 Hz, 2H), 3.18 (t, J=4.67 Hz, 1H), 4.04 (d, J=4.55 Hz, 2H), 6.93-7.17 (m, 2H), 7.27-7.46 (m, 2H).

### Preparation of intermediate 52: 1-chloro-3-(4-chlorophenyl)butan-2-one

To 2-(4-chlorophenyl)propanoic acid (2.4 g, 13.0 mmol) in 50 mL of THF was added oxalyl chloride (1.23 mL, 14.3 mmol) and two drops of DMF at 25° C. The resulting mixture was stirred for 1.5 hour and concentrated to give the acid chloride as a light yellow oil. The light yellow oil was dissolved in 20 mL of THF and added dropwise to 40 mL of diazomethane in diethyl ether (prepared according to the method described in Org. Syn. Coll., 1943, 2: 165) in a 250 mL Erlenmeyer flask at 0° C. The flask was covered with a piece of aluminum foil loosely. The mixture was stirred gently overnight at 25° C. HCl gas was bubbled into the reaction mixture at 0° C. for 5 minutes. The resulting solution was stirred at 0° C. for 1 hour and concentrated to yield an oily residue, which was transferred to a filter funnel loaded with silica gel and eluted with 150 mL of a mixture of ethyl acetate/hexane (1:4). The filtrate was concentrated to give 1-chloro-3-(4-chlorophenyl)butan-2-one, intermediate 52, as a light yellow oil. ¹H NMR (400 MHz, CDCl₃) δ 1.44 (d, J=7.1 Hz, 3H), 3.67 (s, 2H), 4.04 (q, J=7.1 Hz, 1H), 7.10-7.52 (m, 4H).

### Preparation of intermediate 53: 3-(4-chlorophenyl)-2-oxobutyl acetate

The above oil was dissolved in 50 mL of acetone and cooled to 0° C. Acetic acid (0.89 mL, 15.6 mmol) and triethylamine (2.17 mL, 15.6 mmol) were added. The resulting mixture was warmed to 25° C. and stirred for 2 days. The white precipitates were removed via filtration. The filtrate was concentrated to yield an oily residue, which was purified by column chromatography (silica gel, ethyl acetate:hexane=1:5) afforded the desired product (intermediate 53, 1.7 g, 54% yield) as a light yellow oil. ¹H NMR (400 MHz, CDCl₃) δ 1.42 (d, J=7.3 Hz, 3H), 2.12 (s, 3H), 3.81 (q, J=7.3 Hz, 1H), 4.53 (d, J=17.1 Hz, 1H), 4.68 (d, J=17.1 Hz, 1H), 7.16 (d, J=8.0 Hz, 2H), 7.32 (d, J=8.0 Hz, 2H).

### Preparation of intermediate 54: 7-(thiophen-3-yl)indoline-2,3-dione

The procedure described for the synthesis of intermediate 11 was followed, reacting 7-iodoindoline-2,3-dione (10, 2.0 g, 7.33 mmol) with tetrakis[triphenylphosphine]palladium (0.424 g, 0.367 mmol), followed by 3-thiopheneboronic acid (Aldrich, 1.03 g, 8.06 mmol). Crude 54 was purified by flash chromatography over silica gel (3% ethyl acetate in dichloromethane) to afford bright red crystalline material (54% yield). ¹H NMR (400 MHz, DMSO-d₆) δ 7.15 (t, 1H), 7.36 (dd, J=4.9, 1.4 Hz, 1H), 7.50 (dt, J=7.3, 1.0 Hz, 1H), 7.68 (d, J=1.5 Hz, 1H), 7.71 (m, 2H), 7.75 (dd, J=2.9, 1.4 Hz, 1H), 10.86 (s, 1H).

### Preparation of intermediate 55: 2-(1-(4-chlorophenyl)cyclopropyl)-2-oxoethyl acetate

Intermediate 55 was synthesized following the procedure used for intermediate 40, reacting 1-(4-chlorophenyl)cyclopropanecarboxylic acid (2.4 g, 12.2 mmol) with oxalyl chloride (1.15 mL, 13.4 mmol) to give 2-chloro-1-(1-(4-chlorophenyl)cyclopropyl)ethanone, which was reacted with acetic acid (1.78 mL, 31.2 mmol) and triethylamine (4.34 mL, 31.2 mmol) to yield the desired product (1.4 g, 46% yield) as a light yellow oil.

2-Chloro-1-(1-(4-chlorophenyl)cyclopropyl)ethanone. ¹H NMR (400 MHz, CDCl₃) δ 1.26 (dd, J=7.1, 3.4 Hz, 2H), 1.74 (dd, J=7.1, 3.4 Hz, 2H), 4.08 (s, 2H), 7.34-7.36 (m, 4H).

2-(1-(4-Chlorophenyl)cyclopropyl)-2-oxoethyl acetate (intermediate 55). ¹H NMR (400 MHz, CDCl₃) δ 1.21 (dd, J=6.6, 3.4 Hz, 2H), 1.70 (dd, J=6.6, 3.4 Hz, 2H), 2.11 (s, 3H), 4.54 (s, 2H), 7.33-7.40 (m, 4H).

### Preparation of intermediate 56: 3-(4-chlorophenyl)-3-methyl-2-oxobutyl acetate

Intermediate 56 was synthesized following the procedure used for intermediate 40, reacting 2-(4-chlorophenyl)-2-methylpropanoic acid (5.9 g, 29.8 mmol) with oxalyl chloride (2.6 mL, 32.8 mmol) to give 1-chloro-3-(4-chlorophenyl)-3-methylbutan-2-one, which was reacted with acetic acid (2.67 mL, 46.8 mmol) and triethylamine (6.51 mL, 46.8 mmol) to yield the desired product (0.7 g, 9.2% yield) as a colorless oil.

### 1-Chloro-3-(4-chlorophenyl)-3-methylbutan-2-one. ¹H NMR (400 MHz, CDCl₃) δ 1.54 (s, 6H), 4.02 (s, 2H), 7.27 (d, J=8.9 Hz, 2H), 7.37 (d, J=8.9 Hz, 2H).

3-(4-Chlorophenyl)-3-methyl-2-oxobutyl acetate (intermediate 56). ¹H NMR (400 MHz, CDCl₃) δ 1.53 (s, 6H), 2.11 (s, 3H), 4.56 (s, 2H), 7.20-7.37 (m, 4H).

### Preparation of intermediate 57: 1-hydroxy-3-phenylpentan-2-one

A mixture of 2-phenylbutanoic acid (2.0 g, 12.2 mmol) and 7 mL of thionyl chloride in 15 mL of toluene was heated at 115° C. for 16 hours. Concentration of the reaction mixture gave an oily residue. To this residue was added 10 mL of toluene and the resulting mixture was concentrated to yield a yellow oil. 1,1,2-tris(trimethylsilyloxy)ethane (8.0 mL, 24.4 mmol) was added to the yellow oil. The reaction mixture was heated at 100° C. for 16 hours under nitrogen atmosphere. At 50° C., 10 mL of dioxane and 2 mL of 1N HCl were added. The resulting mixture was stirred at 80° C. for 2 hours. Concentration of the mixture gave a yellow oily residue. 10 mL of water and 15 mL of diethyl ether were added. The organic layer was washed with 5 mL each of saturated sodium bicarbonate solution and brine, and dried over magnesium sulfate. The solid was removed via filtration. Concentration of the filtrate afforded the desired product (intermediate 57, 1.74 g, 80% yield) as a yellow oil, which was used for the next step without further purification. ¹H NMR (400 MHz, CDCl₃) δ 0.85 (t, J=7.2 Hz, 3H), 1.77-1.88 (m, 1H), 2.09-2.17 (m, 1H), 3.52 (t, J=7.2 Hz, 1H), 4.21 (d, J=4.9 Hz, 2H), 7.18-7.37 (m, 5H).

### Preparation of intermediate 58: 1-(1,2-dihydrocyclobutabenzen-1-yl)-2-hydroxyethanone

Intermediate 58 was synthesized following the procedure used for intermediate 57, reacting 1-benzocyclobutenecarboxylic acid (1.0 g, 6.76 mmol) with 3.5 mL of thionyl chloride and 1,1,2-tris(trimethylsilyloxy)ethane (4.4 mL, 13.34 mmol) to yield the desired product (0.55 g, 65% yield) as a colorless oil. ¹H NMR (400 MHz, CDCl₃) δ 2.82-2.98 (m, 1H), 3.05-3.20 (m, 1H), 3.46-3.51 (m, 1H), 4.44-4.47 (m, 2H), 7.05-7.81 (m, 4H).

### Preparation of intermediate 59: 1-hydroxy-4-methyl-3-phenylpentan-2-one

Intermediate 59 was synthesized following the procedure used for intermediate 57, reacting 3-methyl-2-phenylbutanoic acid (1.0 g, 5.60 mmol) with 3.5 mL of thionyl chloride and 1,1,2-tris(trimethylsilyloxy)ethane (3.7 mL, 11.2 mmol) to yield the desired product (0.65 g, 60% yield) as a colorless oil. ¹H NMR (400 MHz, CDCl₃) δ 0.71 (d, J=6.8 Hz, 3H), 0.98 (d, J=6.8 Hz, 3H), 2.43-2.55 (m, 1H), 3.26 (d, J=10.7 Hz, 1H), 4.18 (d, J=19.2 Hz, 1H), 4.27 (d, J=19.2 Hz, 1H), 7.21-7.34 (m, 5H).

### Preparation of intermediate 60: 1-hydroxy-3-methyl-4-phenylbutan-2-one

Intermediate 60 was synthesized following the procedure used for intermediate 57, reacting 2-methyl-3-phenylpropanoic acid (1.0 g, 6.1 mmol) with 3.5 mL of thionyl chloride and 1,1,2-tris(trimethylsilyloxy)ethane (4.0 mL, 12.2 mmol) to yield the desired product (0.70 g, 64% yield) as a colorless oil. ¹H NMR (400 MHz, CDCl₃) δ 1.16 (d, J=7.0 Hz, 3H), 2.68 (dd, J=13.3, 7.0 Hz, 1H), 2.76-2.89 (m, 1H), 2.99 (dd, J=13.3, 7.6 Hz, 1H), 3.94 (dd, J=19.3, 4.2 Hz, 1H), 4.24 (dd, J=19.3, 4.2 Hz, 1H), 7.18-7.32 (m, 5H).

### Preparation of intermediate 61: hydroxy-4-phenylpentan-2-one

Intermediate 61 was synthesized following the procedure used for intermediate 44, reacting 3-phenylbutanoic acid (1.0 g, 6.1 mmol) with 3.5 mL of thionyl chloride and 1,1,2-tris(trimethylsilyloxy)ethane (4.0 mL, 12.2 mmol) to yield the desired product (0.80 g, 74% yield) as a colorless oil. ¹H NMR (400 MHz, CDCl₃) δ 1.30 (d, J=7.0 Hz, 3H), 2.64 (dd, J=15.7, 7.1 Hz, 1H), 2.73 (dd, J=15.7, 7.1 Hz, 1H), 3.01 (t, J=4.4 Hz, 1H), 3.30-3.42 (m, 1H), 4.01 (dd, J=19.2, 4.4 Hz, 1H), 4.14 (dd, J=19.2, 4.4 Hz, 1H), 7.17-7.34 (m, 5H).

### Preparation of intermediate 62: N-(2-ethylphenyl)-2-(hydroxyimino)acetamide

The procedure described above for the first step of intermediate 3 was followed, reacting 2-ethylaniline (2.0 mL, 2.0 g, 16.5 mmol) with chloral hydrate (3.28 g, 19.8 mmol), hydroxylamine hydrochloride (4.13 g, 59.4 mmol) and sodium sulfate (23 g, 165 mmol) to give a lumpy brown precipitate.

### Preparation of intermediate 63: 7-ethylindoline-2,3-dione

The procedure described by Yang et al. (see J. Am. Chem. Soc., 1996, 118: 9557) was followed. Intermediate 62 was pulverized and added in small portions, with stirring, to 15 mL of concentrated sulfuric acid that had been heated to 90° C. in a 50 mL Erlenmeyer flask. The acetamide was added slowly to keep the temperature of the reaction mixture below 105° C. After the addition was complete, the purplish-black solution was allowed to stir at 90° C. for 15 minutes, cooled to 60° C., and poured onto 15 g of crushed ice in a beaker. Additional ice was added until the outside of the beaker felt cold to touch. The orange-brown precipitate was collected by filtration and dried under vacuum overnight to yield indoline-2,3-dione which was pure enough to use in the next step (intermediate 63, 0.77 g, 27% yield). Intermediate 63 could also be recrystallized from ethanol to yield pure product as orange-red needles. ¹H NMR (400 MHz, DMSO-d₆) δ 1.14 (t, J=7.5 Hz, 3H), 2.56 (q, J=7.6 Hz, 2H), 7.03 (t, J=7.5 Hz, 1H), 7.35 (d, J=7.3 Hz, 1H), 7.46 (d, J=7.6 Hz, 1H), 11.11 (s, 1H).

### Preparation of intermediate 64: N-(2-sec-butylphenyl)-2-(hydroxyimino)acetamide

The procedure described above for the first step of intermediate 3 was followed, reacting 2-sec-butylaniline (10.4 mL, 10 g, 67 mmol) with chloral hydrate (13.3 g, 80.4 mmol), hydroxylamine hydrochloride (16.8 g, 0.241 mol), and sodium sulfate (76 g, 0.54 mol). Product did not precipitate in solid form, so the cooled reaction mixture was extracted with three portions of ethyl acetate, and the ethyl acetate solution was washed with brine, dried over anhydrous magnesium sulfate, filtered, and concentrated under reduced pressure to yield intermediate 64 as a sticky dark brown oil of sufficient purity to be used in the cyclization step. ¹H NMR (400 MHz, DMSO-d₆) δ 0.75 (t, J=7.3 Hz, 3H), 1.14 (d, J=6.8 Hz, 3H), 1.51 (m, 2H), 2.86 (m, 1H), 7.24 (m, 4H), 7.68 (s, 1H), 9.57 (s, 1H), 12.16 (s, 1H).

### Preparation of intermediate 65: 7-sec-butylindoline-2,3-dione

To carry out the cyclization, 50 mL of concentrated sulfuric acid were added to a round-bottom flask containing intermediate 64, and the mixture was heated with stirring, open to air, to 80° C. for 30 minutes. The resulting mixture was cooled to room temperature, poured onto 250 mL of crushed ice, and allowed to stand for 30 minutes. The precipitate was collected by filtration, washed three times with water, and dried under vacuum to yield indoline-2,3-dione of sufficient purity to use in the next step (intermediate 65, 7.03 g, 52% yield from 2-sec-butylaniline). ¹H NMR (400 MHz, DMSO-d₆) δ 0.81 (t, J=7.3 Hz, 3H), 1.17 (d, J=6.8 Hz, 3H), 1.55 (m, 2H), 2.83 (m, 1H), 7.06 (t, J=7.6 Hz, 1H), 7.36 (d, J=7.1 Hz, 1H), 7.51 (d, J=7.6 Hz, 1H), 11.09 (s, 1H).

### Preparation of intermediate 66: N-(2-tert-butylphenyl)-2-(hydroxyimino)acetamide

The procedure described above for the first step of intermediate 3 was followed, reacting 2-tert-butylaniline (10.4 mL, 10.0 g, 67.0 mmol) with chloral hydrate (13.3 g, 80.4 mmol), hydroxylamine hydrochloride (16.8 g, 0.241 mol), and sodium sulfate (114 g, 0.804 mol). Ethyl acetate extraction of the cooled reaction mixture gave, after evaporation, crude acetamide of sufficient purity to be used in the next step (intermediate 66, 13.6 g, 92% yield).

### Preparation of intermediate 67: 7-tert-butylindoline-2,3-dione

The procedure described above for intermediate 65 was followed, heating intermediate 66 with 45 mL of concentrated sulfuric acid. Indoline-2,3-dione of sufficient purity to be used in the next step was obtained (intermediate 67, 6.92 g, 55% yield). ¹H NMR (400 MHz, DMSO-d₆) δ 1.32 (s, 9H), 7.04 (t, 1H), 7.39 (d, J=7.3 Hz, 1H), 7.55 (dd, J=7.8, 1.3 Hz, 1H), 10.76 (s, 1H).

### Preparation of intermediate 68: N-(2-fluorophenyl)-2-(hydroxyimino)acetamide

The procedure described above for the first step of intermediate 3 was followed, reacting 2-fluoroaniline (8.7 mL, 10 g, 90 mmol) with chloral hydrate (17.9 g, 0.108 mol) and hydroxylamine hydrochloride (22.5 g, 0.324 mol) in the presence of sodium sulfate (128 g, 0.900 mol). Pure intermediate 68 was collected by filtration and dried under vacuum (11.7 g, 71% yield). ¹H NMR (400 MHz, DMSO-d₆) δ 7.20 (m, 2H), 7.29 (m, 1H), 7.74 (s, 1H), 7.86 (m, 1H), 9.81 (s, 1H), 12.30 (s, 1H).

### Preparation of intermediate 69: 7-fluoroindoline-2,3-dione

The procedure described above for intermediate 63 was followed, heating intermediate 68 (11.7 g) in 60 mL of concentrated sulfuric acid. The indoline-2,3-dione obtained was of sufficient purity to be used directly in the next step (intermediate 69, 6.87 g, 65% yield). ¹H NMR (400 MHz, DMSO-d₆) δ 7.08 (ddd, 1H), 7.38 (dt, J=7.5, 0.8 Hz, 1H), 7.54 (ddd, J=10.4, 8.3, 1.0 Hz, 1H), 11.56 (s, 1H).

### Preparation of intermediate 70: N-(2-bromophenyl)-2-(hydroxyimino)acetamide

The procedure described above for the first step of intermediate 3 was followed, reacting 2-bromoaniline (10 g, 58 mmol) with chloral hydrate (11.5 g, 69.7 mmol) and hydroxylamine hydrochloride (14.5 g, 0.209 mol) in the presence of sodium sulfate (99 g, 0.70 mol). The lumpy brown precipitate was collected by filtration and dried under vacuum (intermediate 70, 11.98 g, 85% yield). ¹H NMR (400 MHz, DMSO-d₆) δ 7.16 (t, 1H), 7.41 (t, J=7.7 Hz, 1H), 7.69 (m, 2H), 7.91 (d, J=8.1 Hz, 1H), 9.46 (s, 1H), 12.45 (s, 1H).

### Preparation of intermediate 71: 7-bromoindoline-2,3-dione

The procedure described above for intermediate 13 was followed, heating intermediate 70 (3.11 g, 12.8 mmol) in 10 mL of concentrated sulfuric acid to give a reddish-brown powder (intermediate 71, 2.22 g, 77% yield). ¹H NMR (400 MHz, DMSO-d₆) δ 7.02 (t, J=7.8 Hz, 1H), 7.52 (d, J=6.6 Hz, 1H), 7.79 (d, J=8.1 Hz, 1H), 11.32 (s, 1H).

### Preparation of intermediate 72: 2-(hydroxyimino)-N-(2-methylphenyl)acetamide

The procedure described above for the first step of intermediate 3 was followed, reacting o-toluidine (10 mL, 10 g, 93 mmol) with chloral hydrate (19 g, 0.11 mol) and hydroxylamine hydrochloride (23 g, 0.34 mol) in the presence of sodium sulfate (133 g, 0.933 mol), to give intermediate 72 as a fluffy, off-white powder (10.9 g, 65% yield).

### Preparation of intermediate 73: 7-methylindoline-2,3-dione

The procedure described above for intermediate 13 was followed, heating intermediate 72 in 45 mL of concentrated sulfuric acid to give an orange powder (intermediate 73, 5.96 g, 61% yield). ¹H NMR (400 MHz, DMSO-d₆) δ 2.19 (s, 3H), 6.99 (t, J=7.6 Hz, 1H), 7.34 (d, J=7.6 Hz, 1H), 7.43 (d, J=7.6 Hz, 1H), 11.09 (s, 1H).

### Preparation of intermediate 74: 2-(hydroxyimino)-N-(3-methylphenyl)acetamide

The procedure described above for the first step of intermediate 3 was followed, reacting m-toluidine (10 mL, 10 g, 93 mmol) with chloral hydrate (19 g, 0.11 mol) and hydroxylamine hydrochloride (23 g, 0.34 mol) in the presence of sodium sulfate (133 g, 0.933 mol), to give intermediate 74 (14.4 g, 87% yield).

### Preparation of intermediates 75 and 76: 6-methylindoline-2,3-dione/4-methylindoline-2,3-dione

The procedure described above for intermediate 13 was followed, heating intermediate 74 in 60 mL of concentrated sulfuric acid to give an inseparable mixture of 6-methylisatin and 4-methylisatin, an orange powder (intermediates 75 and 76, 3.44 g, 26% yield). ¹H NMR (400 MHz, DMSO-d₆) δ 2.35 (s, 1.5H), 2.44 (s, 1.5H), 6.71 (m, 1H), 6.87 (t, 1H), 7.42 (m, 1H), 10.99 (s, 1H).

### Preparation of Intermediate 77: 2-hydroxy-1-(1-p-tolyl-cyclopropyl)-ethanone

Intermediate 77 was prepared following the procedure for intermediate 51, using as starting material 1-p-tolyl-cyclopropanecarboxylic acid. The crude mixture was taken forward to the next step.

### Preparation of Intermediate 78: 1-(1-(4-chlorophenyl)cyclopropyl)-2-hydroxyethanone

In a 1 L round-bottom flask, 1-(4-chlorophenyl)cyclopropanecarboxylic acid (20 g, 0.10 mol) was taken up in 175 mL of toluene. Thionyl chloride (75 mL, 122 g, 1.0 mol) was added and the solution was heated at reflux temperature overnight under nitrogen. After cooling, toluene and excess thionyl chloride were removed by evaporation and azeotroping with three additional 100 mL-portions of toluene. The acid chloride was heated overnight at 100° C. with tris(trimethylsiloxy)ethylene (67 mL, 59 g, 0.20 mol) under nitrogen. The reaction mixture was subsequently cooled to 50° C. and diluted with 100 mL of 1,4-dioxane and 20 mL of 1 M hydrochloric acid. The resulting mixture was heated at 80° C. for 2 hours. The organic solvents were removed under reduced pressure and the remaining mixture was diluted with 150 mL of water and extracted with three portions of diethyl ether. The combined organic layers were washed with two portions of 5% sodium carbonate solution, dried over anhydrous magnesium sulfate, filtered, and concentrated to give a yellow oil (intermediate 78, 17.9 g, 83% yield). This could be further purified by flash chromatography over silica gel (6-50% ethyl acetate in hexanes). ¹H NMR (400 MHz, CDCl₃) δ 1.28 (q, J=4.0 Hz, 2H), 1.74 (q, J=3.5 Hz, 2H), 3.16 (t, J=4.7 Hz, 1H), 4.05 (d, J=4.8 Hz, 2H), 7.29-7.32 (m, 2H), 7.33-7.37 (m, 2H).

### Preparation of Intermediate 79: iodo-7-(trifluoromethyl)indoline-2,3-dione

The iodination methodology described by C. Lamas, J. Barluenga et al. (see J. Org. Chem., 1996, 61: 5804) was followed. Intermediate 6 (8.79 g, 40.9 mmol) was taken up in 105 mL of anhydrous dichloromethane in a 500 mL round-bottom flask. Bis(pyridine)iodonium(I) tetrafluoroborate (23 g, 61 mmol) was added, followed by trifluoromethanesulfonic acid (10.8 mL, 18.4 g, 0.123 mol). The mixture was stirred at room temperature for 40 minutes, until LC-MS analysis showed complete disappearance of starting material. The solution was treated with 105 mL of water and extracted with two 45 mL-portions of dichloromethane. The combined organic layers were washed with 5% aqueous sodium thiosulfate, dried over anhydrous magnesium sulfate, filtered, and concentrated to give pure product (intermediate 79, 12.0 g, 87% yield). ¹H NMR (400 MHz, DMSO-d₆) δ 8.03 (s, 1H), 8.11 (s, 1H), 11.55 (s, 1H).

### Preparation of Intermediate 80: 5-methyl-7-(trifluoromethyl)indoline-2,3-dione

The procedure described by Lisowski et al. (see J. Org. Chem., 2000, 65: 4193) was followed. Intermediate 79 (1.12 g, 3.28 mmol) and tetrakis(triphenylphosphine)palladium (190 mg, 0.16 mmol) were taken up in 100 mL of ethylene glycol dimethyl ether in a 500 mL round-bottom flask. This solution was purged three times by opening to vacuum followed by backfilling with nitrogen. Methylboronic acid (390 mg, 6.6 mmol) was added, followed by a solution of sodium bicarbonate (0.55 g, 6.6 mmol) in 100 mL of water, and the evacuation/nitrogen backfill procedure was repeated once more. The mixture was heated at reflux temperature and monitored for product appearance/starting material disappearance by LC-MS analysis. After 1.5 hours, an additional 190 mg (0.16 mmol) of the palladium catalyst was added and the reaction allowed to be heated at reflux temperature overnight. The organic solvent was removed and the remaining aqueous mixture was partitioned between 100 mL each of 2 M hydrochloric acid and ethyl acetate. The aqueous layer was extracted with additional ethyl acetate and the combined organic layers were washed with brine, dried over anhydrous magnesium sulfate, filtered, and concentrated to give the crude product, which was purified by flash chromatography over silica gel (0-6% ethyl acetate in dichloromethane) to give intermediate 80 of sufficient purity (the product contained about 20% of the deiodinated side-product, 7-(trifluoromethyl)isatin). ¹H NMR (400 MHz, DMSO-d₆) δ 3.33 (s, 3H), 7.62 (s, 1H), 7.68 (s, 1H), 11.35 (s, 1H).

### Preparation of Intermediate 81: N-(4-chloro-2-(trifluoromethyl)phenyl)-2-(hydroxyimino)acetamide

The methodology reported by L. Kuyper et al. (see J. Med. Chem. 2001, 44: 4339) was used. In a 1 L round-bottom flask, anhydrous sodium sulfate (85 g) was dissolved in 230 mL of boiling water, with stirring. A hot solution of 4-chloro-2-(trifluoromethyl)aniline (6.5 g, 33 mmol) in 50 mL of 1 M hydrochloric acid, 2 mL of concentrated hydrochloric acid and 30 mL of ethanol was added. An additional 60 mL of ethanol was added. Chloral hydrate (6.6 g, 40 mmol) was added, followed by hydroxylamine hydrochloride (7.6 g, 0.11 mol) in 30 mL of water. The mixture was heated at reflux temperature and ethanol was added until the aniline was dissolved again. Heating was continued for 3 hours. With the flask open to atmosphere, the reaction mixture was heated at reflux temperature overnight. The reaction mixture was cooled to 0° C. and the off-white precipitate was collected by filtration. This precipitate, which contained a large amount of sodium sulfate, was taken up in 300 mL of water, stirred at room temperature for 1 hour, filtered, taken up in 200 mL of water, stirred for 30 minutes, filtered, and dried under vacuum to give an off-white powder (intermediate 81, 2.65 g, 30% yield). ¹H NMR (400 MHz, DMSO-d₆) δ 7.66 (s, 1H), 7.76-7.86 (m, 3H), 9.63 (s, 1H), 12.44 (s, 1H).

### Preparation of Intermediate 82: 5-chloro-7-(trifluoromethyl)indoline-2,3-dione

The procedure of M. Kollmar et al. (see Org. Synth., “2-Amino-3-fluorobenzoic acid”) was followed. In a 50 mL Erlenmeyer flask, 4 mL of concentrated sulfuric acid was heated to 70° C., with stirring. Intermediate 81 was added gradually, maintaining the temperature below 90° C. The reaction mixture was heated at 90° C. for an additional hour. It was cooled rapidly to 20° C., poured to a vigorously stirred mixture of 35 mL of ice water and 7 mL of ethyl acetate. Once all the ice had melted, the layers were separated, and the aqueous layer was extracted with additional ethyl acetate. The combined organic layers were washed with brine, dried over anhydrous magnesium sulfate, filtered, and concentrated to give a brownish-black solid, which was purified by flash chromatography over silica gel (0-6% ethyl acetate in dichloromethane) to give intermediate 82 of sufficient purity (0.633 g, 42% yield). ¹H NMR (400 MHz, DMSO-d₆) δ 7.87 (d, J=2.0 Hz, 1H), 7.94 (d, J=2.0 Hz, 1H), 11.58 (s, 1H).

### Preparation of Intermediate 83: 5-phenyl-7-(trifluoromethyl)indoline-2,3-dione

The procedure described above for the synthesis of intermediate 80 was followed, reacting intermediate 77 (2.0 g, 5.9 mmol) with phenylboronic acid (0.79 g, 6.5 mmol) in the presence of tetrakis(triphenylphosphine)palladium (339 mg, 0.29 mmol) and sodium bicarbonate (0.98 g, 12 mmol). LC-MS analysis showed complete disappearance of starting material after 1 hour. After 2 hours, the reaction mixture was cooled to room temperature and worked up as described above to give pure product (intermediate 83, 0.98 g, 57% yield). ¹H NMR (400 MHz, DMSO-d₆) δ 7.41 (t, J=7.2 Hz, 1H), 7.49 (t, J=7.6 Hz, 2H), 7.75 (ddd, J=7.6, 2.2, 1.9 Hz, 2H), 8.06 (d, J=4.6 Hz, 2H), 11.56 (s, 1H).

### Preparation of Intermediate 84: tert-butyl-4-(trifluoromethyl)phenylcarbamate

In a 250 mL round-bottom flask, 4-(trifluoromethyl)aniline (7.7 mL, 10 g, 62 mmol) and di-tert-butyldicarbonate (13.6 g, 62.1 mmol) were taken up in 60 mL of anhydrous tetrahydrofuran and was heated at reflux temperature overnight. After cooling to room temperature, the solvent was removed and the residue was taken up in 250 mL of ethyl acetate. This solution was washed with three 125 mL-portions of 0.5 M citric acid and 125 mL of brine, dried over anhydrous magnesium sulfate, filtered, and concentrated. The crude product, a white solid, was purified by flash chromatography over silica gel (2-20% ethyl acetate in hexanes) to give a fluffy white solid (intermediate 84, 14.4 g, 89% yield). ¹H NMR (400 MHz, DMSO-d₆) δ 1.49 (s, 9H), 7.59-7.63 (m, 2H), 7.64-7.68 (m, 2H), 9.79 (s, 1H).

### Preparation of Intermediate 85: ethyl 2-(2-tert-butoxycarbonylamino)-5-(trifluoromethyl)phenyl)-2-oxoacetate

The procedure described by Hewawasam et al (see Tetrahedron Lett. 1994, 35: 7303) was followed. Intermediate 84 (9.62 g, 36.8 mmol) was placed in a 500 mL round-bottom flask, azeotroped with hexanes, and dried under vacuum overnight. Then, under nitrogen atmosphere, 55 mL of anhydrous tetrahydrofuran was added by syringe and the solution cooled to −78° C. (dry ice/acetone). A solution of sec-butyllithium in cyclohexane (1.4 M, 63 mL, 88 mmol) was added in rapid drops via syringe. The reaction mixture was warmed to 40° C. (dry ice/acetonitrile) for 2 hours. After the resulting mixture was cooled to −78° C., diethyl oxalate (6.0 mL, 6.5 g, 49 mmol) was added rapidly in one portion by syringe. The reaction mixture was allowed to stir at −78° C. for 45 minutes, and was quenched with 15 mL of 1 M hydrochloric acid. Additional hydrochloric acid was added until the mixture was acidic and the resulting mixture was extracted with two portions of diethyl ether. The combined ether layers were washed with brine, dried over anhydrous magnesium sulfate, filtered, concentrated, and purified by flash chromatography over silica gel (1-10% ethyl acetate in hexanes) to give a viscous light yellow oil (intermediate 85, 4.46 g, 34% yield). ¹H NMR (400 MHz, DMSO-d₆) δ 1.28 (t, J=7.2 Hz, 3H), 1.44 (s, 9H), 4.28 (q, J=7.2 Hz, 2H), 7.69 (d, J=8.3 Hz, 1H), 7.91 (d, J=2.0 Hz, 1H), 7.92-7.96 (m, 1H), 10.18 (s, 1H).

### Preparation of Intermediate 86: 5-(trifluoromethyl)indoline-2,3-dione

The procedure described by Hewawasam et al (see Tetrahedron Lett., 1994, 35: 7303) was followed. Intermediate 85 was taken up in 90 mL each of tetrahydrofuran and 3 M hydrochloric acid, and the solution was heated at reflux temperature overnight, until LC-MS and t.l.c. analysis (5% ethyl acetate in dichloromethane) showed complete conversion to product. Upon removal of the organic solvent, the product precipitated out of solution. Solids were collected by filtration, washed with water, and dried under vacuum to give fluffy, bright yellow crystals (intermediate 86, 2.22 g, 85% yield). ¹H NMR (400 MHz, DMSO-d₆) δ 7.08 (d, J=8.3 Hz, 1H), 7.81 (s, 1H), 7.90-7.95 (m, 1H), 11.39 (s, 1H).

### Preparation of Intermediate 87: 7-iodo-5-(trifluoromethyl)indoline-2,3-dione

The procedure described above for intermediate 77 was followed, reacting intermediate 86 (2.22 g, 10.3 mmol) with bis(pyridine)iodonium(I) tetrafluoroborate (5.75 g, 15.5 mmol) in the presence of trifluoromethanesulfonic acid (2.7 mL, 4.6 g, 31 mmol), to give pure product as a bright yellow powder (intermediate 87, 3.27 g, 93% yield). ¹H NMR (400 MHz, DMSO-d₆) δ 7.80 (s, 1H), 8.28 (dd, J=1.8, 0.8 Hz, 1H), 11.38 (s, 1H).

### Preparation of Intermediate 88: 7-methyl-5-(trifluoromethyl)indoline-2,3-dione

The procedure described above for intermediate 80 was followed, reacting intermediate 87 (0.746 g, 2.19 mmol) with methylboronic acid (0.26 g, 4.4 mmol) in the presence of tetrakis(triphenylphosphine)palladium (127 mg, 0.110 mmol) and sodium bicarbonate (0.37 g, 4.4 mmol). After the reaction mixture was heated at reflux temperature overnight, additional aliquot of palladium catalyst (127 mg, 0.110 mmol) was added, and the reaction mixture was heated at reflux temperature for additional 5 hours. It was worked up and purified as described above, to give product of sufficient purity (intermediate 88, 0.259 g, 52% yield). ¹H NMR (400 MHz, DMSO-d₆) δ 2.27 (s, 3H), 7.63 (s, 1H), 7.82 (s, 1H), 11.44 (s, 1H).

### Preparation of Intermediate 89: 5-ethyl-7-(trifluoromethyl)indoline-2,3-dione

The procedure described above for intermediate 80 was followed, reacting intermediate 77 (1.47 g, 4.30 mmol) with a solution of triethylborane in tetrahydrofuran (1.0 M, 8.6 mL, 8.6 mmol) in the presence of dichloro[1,1′-bis(diphenylphosphino)ferrocene]palladium (II) dichloromethane adduct (176 mg, 0.215 mmol) and cesium carbonate (4.20 g, 12.9 mmol). Flash chromatography over silica gel (0-6% ethyl acetate in dichloromethane) gave product of sufficient purity (intermediate 89, 0.417 g, 40% yield). ¹H NMR (400 MHz, DMSO-d₆) δ 1.17 (t, J=7.6 Hz, 3H), 2.65 (q, J=7.6 Hz, 2H), 7.66 (s, 1H), 7.69 (s, 1H), 11.35 (s, 1H).

### Preparation of Intermediate 90: 7-ethyl-5-(trifluoromethyl)indoline-2,3-dione

The procedure described above for intermediate 80 was followed, reacting intermediate 87 (1.60 g, 4.70 mmol) with a solution of triethylborane in tetrahydrofuran (1.0 M, 9.4 mL, 9.4 mmol) in the presence of dichloro[1,1′-bis(diphenylphosphino)ferrocene]palladium (ii) dichloromethane adduct (192 mg, 0.235 mmol) and cesium carbonate (4.58 g, 14.1 mmol). The crude product was purified by flash chromatography over silica gel (1-10% ethyl acetate in dichloromethane) to give a yellow-orange solid (intermediate 90, 0.439 g, 38% yield). ¹H NMR (400 MHz, DMSO-d₆) δ 1.16 (t, J=7.5 Hz, 3H), 2.64 (q, J=7.6 Hz, 2H), 7.65 (s, 1H), 7.80 (d, J=0.8 Hz, 1H), 11.45 (s, 1H).

### Preparation of Intermediate 91: 8-phenyl-5-(trifluoromethyl)indoline-2,3-dione

The procedure described above for intermediate 80 was followed, reacting intermediate 87 (1.60 g, 4.70 mmol) with phenylboronic acid (0.63 g, 5.2 mmol) in the presence of tetrakis(triphenylphosphine)palladium (272 mg, 0.235 mmol) and sodium bicarbonate (0.79 g, 9.4 mmol). The crude product was purified by flash chromatography over silica gel (0-6% ethyl acetate in dichloromethane) to give a yellow-orange solid (0.585 g, 43% yield): ¹H NMR (400 MHz, DMSO-d₆) δ 7.45-7.55 (m, 5H), 7.84 (dd, J=11.2, 1.4 Hz, 2H), 11.28 (s, 1H).

### Preparation of Intermediate 92: 1-(1-phenylcyclopropyl)-2-hydroxyethanone

The procedure described above for intermediate 78 was followed, reacting 1-phenylcyclopropanecarboxylic acid (20 g, 0.12 mol) successively with thionyl chloride (90 mL, 150 g, 1.2 mol) and tris(trimethylsiloxy)ethylene (70 mL, 62 g, 0.21 mol). The crude product was purified by flash chromatography over silica gel (2-20% ethyl acetate in hexanes) to give a nearly colorless oil (intermediate 92, 9.44 g, 44% yield). ¹H NMR (400 MHz, CDCl₃) δ 1.31 (q, J=3.7 Hz, 2H), 1.74 (q, J=3.6 Hz, 2H), 3.18 (t, J=4.9 Hz, 1H), 4.06 (d, J=5.1 Hz, 2H), 7.33-7.38 (m, 5H).

### Preparation of Intermediate 93: 5-bromo-7-(trifluoromethyl)indoline-2,3-dione

Intermediate 6 (4.56 g, 21.2 mmol) was taken up in 45 mL of acetic acid in a 250 mL round-bottom flask, and bromine (5.4 mL, 17 g, 0.11 mol) was added. The solution was stirred overnight at room temperature. LC-MS analysis showed that complete conversion to product had not occurred. Additional bromine was added (1.1 mL, 3.4 g, 21 mmol) and the resulting mixture was stirred for additional 5 hours. The reaction mixture was poured onto crushed ice and allowed to stand until the ice had melted. The precipitate was collected by filtration, washed repeatedly with water, and dried under vacuum to give fine, bright orange crystals (intermediate 93, 5.12 g, 82% yield). ¹H NMR (400 MHz, DMSO-d₆) δ 7.96 (d, J=2.0 Hz, 1H), 8.04 (d, J=2.0 Hz, 1H), 11.59 (s, 1H).

### Preparation of Intermediate 94: tert-butyl 2,4-bis(trifluoromethyl)phenylcarbamate

In a 100 mL 2-necked round-bottom flask fitted with a condenser, 2,4-bis(trifluoromethyl)aniline (5.33 g, 23.3 mmol) was taken up in 25 mL of anhydrous tetrahydrofuran. The solution was cooled to 0° C. and sodium hydride in mineral oil was added (1.03 g, 60 wt %, 0.615 g NaH, 25.6 mmol). The mixture was stirred for 30 minutes at 0° C. and di-tert-butyldicarbonate (10.2 g, 46.6 mmol) was added. The reaction mixture was stirred at room temperature for 1.5 hours and was heated at reflux temperature overnight. Flash chromatography over silica gel (0-4% ethyl acetate in hexanes) gave pure material (intermediate 94, 3.14 g, 41% yield). ¹H NMR (400 MHz, DMSO-d₆) δ 1.46 (s, 9H), 7.82 (d, J=8.6 Hz, 1H), 7.99 (s, 1H), 8.04 (dd, J=8.5, 1.6 Hz, 1H), 8.98 (s, 1H).

### Preparation of Intermediate 95: ethyl 2-(2-(tert-butoxycarbonylamino)-3,5-bis(trifluoromethyl)phenyl)-2-oxoacetate

The procedure described above for intermediate 85 was followed, reacting intermediate 94 (3.14 g, 9.54 mmol) with a solution of sec-butyllithium in cyclohexane (1.4 M, 16.3 mL, 22.9 mmol) and diethyl oxalate (1.6 mL, 1.7 g, 11 mmol). Flash chromatography over silica gel (2-20% ethyl acetate in hexanes) gave pure product (intermediate 95, 1.91 g, 47% yield). ¹H NMR (400 MHz, DMSO-d₆) δ 1.29 (t, J=7.1 Hz, 3H), 1.40 (s, 9H), 4.32 (q, J=7.1 Hz, 2H), 8.26 (s, 1H), 8.43 (s, 1H), 9.69 (s, 1H).

### Preparation of Intermediate 96: 5,7-bis(trifluoromethyl)indoline-2,3-dione

The procedure described above for intermediate 86 was followed: hydrolysis of intermediate 95 (1.83 g, 4.27 mmol) gave a bright yellow powder (intermediate 96, 0.931 g, 77% yield). ¹H NMR (400 MHz, DMSO-d₆) δ 8.11 (s, 1H), 8.16 (s, 1H), 11.86 (s, 1H).

### Preparation of Intermediate 97: (1-phenyl-cyclopropyl)-acetic acid

Intermediate 97 was prepared according to the procedure described by Wilt et al. (see J. Org. Chem., 1966, 31: 3018).

### Preparation of Intermediate 98: 1-hydroxy-3-(1-phenyl-cyclopropyl)-propan-2-one

Intermediate 98 was synthesized following the procedure for intermediate 51 reacting (1-phenyl-cyclopropyl)-acetic acid (intermediate 97, 0.3 g, 1.70 mmol) with 3 mL of thionyl chloride and 1,1,2-tris(trimethylsilyloxy)ethane (1.2 mL, 3.40 mmol) to yield the desired product (0.2 g, 62% yield) as a colorless oil. ¹H NMR (400 MHz, CDCl₃) δ 0.91-0.97 (m, 2H), 1.21-1.28 (m, 2H), 2.63 (s, 2H), 4.42 (s, 2H), 7.23-7.39 (m, 5H).

### Preparation of Intermediate 99: 1-benzyl-cyclopropanecarboxylic acid

Intermediate 99 was prepared according to the procedure described by Bartha et al. (see Revue Romaine de Chimie, 1986, 31: 519). A mixture of zinc dust (5.67 g, 86.6 mmol) and cuprous chloride (8.6 g, 86.6 mmol) in 100 mL of diethyl ether was stirred and heated at reflux temperature for 30 minutes under nitrogen. 2-Benzyl-acrylic acid methyl ester (3.85 g, 21.9 mmol) and diiodomethane (2.3 mL, 28.1 mmol, in which 100 mg iodine was dissolved) were quickly added. The reaction mixture was stirred at reflux temperature for 6 hours. At room temperature, saturated ammonium chloride (30 mL) was added. The solid was removed via filtration. The organic layer was separated and the aqueous layer was extracted with two 30 mL-portions of diethyl ether. The combined organic layers were concentrated to give a light yellow oil, which was saponified with potassium hydroxide in methanol. Intermediate 99 was purified via column chromatography (silica gel, ethyl acetate:hexane=1:5). Intermediate 99 (0.9 g, 23% yield) was obtained as a light yellow oil. ¹H NMR (400 MHz, CDCl₃) δ 0.85-0.90 (m, 2H), 1.33-1.37 (m, 2H), 3.62 (s, 2H), 7.14-7.34 (m, 5H).

### Preparation of Intermediate 100: 1-(1-benzyl-cyclopropyl)-2-hydroxy-ethanone

Intermediate 100 was synthesized following the procedure for intermediate 51, reacting 1-benzyl-cyclopropanecarboxylic acid (intermediate 99, 0.9 g, 5.1 mmol) with 5 mL of thionyl chloride and 1,1,2-tris(trimethylsilyloxy)ethane (3.7 mL, 10.2 mmol) to yield the desired product (0.8 g, 82% yield) as a colorless oil. ¹H NMR (400 MHz, CDCl₃) δ 0.87-0.90 (m, 2H), 1.34-1.38 (m, 2H), 3.55 (s, 2H), 3.70-3.71 (s, 2H), 7.15-7.31 (m, 5H).

### Preparation of Intermediate 101: 1-(2-(trifluoromethyl)phenyl)cyclopropane carbonitrile

This compound was prepared following the procedure described by Jonczyk et al. (see Org. Prep. Proc. Int., 1995, 27(3): 355-359). To a 25 mL round-bottom flask equipped with a condenser was added 2-(2-(trifluoromethyl)phenyl)acetonitrile (1.0 g, 5.4 mmol, 1.0 eq.), 1-bromo-2-chloroethane (0.67 mL, 8.1 mmol, 1.5 eq.), and triethylbenzyl ammonium chloride (0.024 g, 0.11 mmol, 0.02 eq.). The resulting mixture was heated to 50° C. and sodium hydroxide (1.3 g, 32.4 mmol, 6.0 eq. dissolved into 1.0 mL of water) was added dropwise. The mixture was stirred at 50° C. for 16 hours, cooled to room temperature, and poured into 50 mL of water. This suspension was extracted with three 25 mL-portions of methylene chloride, and the combined organic layers were washed with three 50 mL-portions of 1.2 N HCl solution, three 50 mL-portions of water, and 50 mL of saturated sodium chloride solution. The organic layer was dried over magnesium sulfate, filtered, and the solvent was removed in vacuo. The crude material was purified by silica gel chromatography (Biotage Flash 40, 10% ethyl acetate/hexanes) to give the desired product as a light yellow oil (intermediate 101, 0.92 g, 81% yield). ¹H NMR (400 MHz, CDCl₃) δ 1.30-1.52 (m, 2H), 1.65-1.86 (m, 2H), 7.42-7.52 (m, 1H), 7.53-7.60 (m, 2H), 7.71 (d, J=7.58 Hz, 1H).

### Preparation of Intermediate 102: 1-(2-(trifluoromethyl)phenyl)cyclopropane carboxylic acid

To a 50 mL round-bottom flask equipped with a condenser was added intermediate 101 (1-(2-(trifluoromethyl)phenyl)cyclopropanecarbonitrile, 0.92 g, 4.4 mmol, 1.0 eq.) and 20 mL of 4.0 N LiOH solution. This suspension was heated at reflux temperature and allowed to stir for 3 days. The resulting mixture was cooled to room temperature and poured into 250 mL of 1.2 N HCl. This suspension was extracted with three 75 mL-portions of ethyl acetate and the combined organic layers were washed with three 200 mL-portions of water and 200 mL of saturated sodium chloride solution. The organic layer was dried over magnesium sulfate, filtered, and solvent was removed in vacuo. The desired product was obtained as a white solid (intermediate 102, 0.87 g, 86% yield). ¹H NMR (400 MHz, CDCl₃) δ 1.18-1.45 (m, 2H). 1.58-1.94 (m, J=81.09 Hz, 2H), 7.31-7.42 (m, 1H), 7.43-7.54 (m, 2H), 7.64 (d, J=7.83 Hz, 1H).

### Preparation of Intermediate 103: 2-chloro-1-(1-(2-(trifluoromethyl)phenyl)cyclopropyl)ethanone

To a 50 mL round-bottom flask equipped with a condenser was added intermediate 102 (1-(2-(trifluoromethyl)cyclopropanecarboxylic acid, 0.83 g, 3.61 mmol, 1.0 eq.) and 25 mL of thionyl chloride. The resulting solution was heated at reflux temperature and allowed to stir for 4 hours. Upon cooling to room temperature, all of the volatiles were removed in vacuo. The resulting brown oil was redissolved into 10 mL of THF and added dropwise to 100 mL of ethereal diazomethane solution cooled to 0° C. This mixture was allowed to warm slowly to room temperature and stir for 12 hours. The solution was cooled back down to 0° C. and HCl gas was bubbled through for 3 minutes. Crushed ice was added to the mixture and stirring was continued for 15 minutes. The layers were separated and the aqueous layer was extracted with two 50 mL-portions of diethyl ether. The combined organic layers were washed with three 100 mL-portions of saturated sodium bicarbonate solution, three 100 mL-portions of water, and 100 mL of saturated sodium chloride solution. The solution was dried over magnesium sulfate, filtered, and the solvent was removed in vacuo to give intermediate 103 as a colorless oil (0.339 g, 36% yield). ¹H NMR (400 MHz, CDCl₃) δ 0.85-1.83 (m, 4H), 3.98 (d, J=6.32 Hz, 2H), 7.42-7.55 (m, 1H), 7.56-7.65 (m, 2H), 7.74 (d, J=7.58 Hz, 1H).

### Preparation of Intermediate 104: 2-oxo-2-(1-(2-(trifluoromethyl)phenyl)cyclopropyl)ethyl acetate

To a 5 mL microwave-reaction vial was added intermediate 103 (2-chloro-1-(1-(2-(trifluoromethyl)phenyl)cyclopropyl)ethanone, 0.339 g, 1.35 mmol, 1.0 eq.) and 2 mL of acetone. To the resulting solution was added acetic acid (0.1 mL, 1.76 mmol, 1.3 eq.) and triethylamine (0.25 mL, 1.76 mmol, 1.3 eq.). The vial was sealed and heated to 150° C. in a microwave reactor for 30 minutes. The resulting suspension was poured into 50 mL of water and extracted with three 25 mL-portions of ethyl acetate. The combined organic layers were washed with three 75 mL-portions water and 75 mL of saturated sodium chloride solution. The organic layer was dried over magnesium sulfate, filtered, and solvent was removed to give a brown oil. This was purified by silica gel chromatography (Biotage Flash 40, 0-10% ethyl acetate/hexanes) to give the desired product as a white solid (intermediate 104, 0.235 g, 64% yield). ¹H NMR (400 MHz, CDCl₃) δ 1.30-1.42 (m, J=12.88 Hz, 2H), 1.44-1.63 (m, 2H), 2.10 (s, 3H), 4.23-4.42 (m, 1H), 4.53-4.72 (m, 1H), 7.45-7.53 (m, 1H), 7.56-7.67 (m, 2H), 7.73 (d, J=8.59 Hz, 1H)

### Preparation of Intermediate 105: 5-isopropylindoline-2,3-dione

Intermediate 105 was synthesized by method used for intermediate 63 using as starting material 4-isopropylaniline in 75% yield. ¹H NMR (400 MHz, DMSO-d₆) δ 1.17 (d, J=6.8 Hz, 6H), 2.81-2.93 (m, 1H), 6.84 (d, J=8.1 Hz, 1H), 7.38 (d, J=1.8 Hz, 1H), 7.49 (dd, J=8.2, 1.9 Hz, 1H), 10.94 (br s, 1H).

### Preparation of Exemplified Compounds

### Compound 1: 2-(1-(4-chlorophenyl)cyclopropyl)-3-hydroxy-8-(trifluoromethoxy)quinoline-4-carboxylic acid

Intermediate 13 (1.00 g, 4.32 mmol) was taken up in 1 mL of ethanol and 3.4 mL of 10 M sodium hydroxide and the resulting mixture was heated at reflux temperature for 20 minutes. A solution of intermediate 55 in 7 mL of ethanol was added dropwise via syringe and the resulting mixture was heated overnight. It was cooled to room temperature and ethanol was removed under reduced pressure. The residue was diluted with water, acidified to pH 1 by slow addition of 1 M hydrochloric acid, and extracted with ethyl acetate. The combined ethyl acetate layers were concentrated to give a dark material which was purified by preparative HPLC (water/acetonitrile with 0.1% triethylamine). The purified triethylammonium salt was taken up in 20% acetonitrile in water and acidified with concentrated hydrochloric acid. Pure product precipitated out of solution as an off-white powder was collected to give Compound 1 (83 mg, 4.5% yield). ¹H NMR (400 MHz, DMSO-d₆) δ 1.35-1.39 (m, 2H), 1.49-1.54 (m, 2H), 7.16 (d, J=8.6 Hz, 2H), 7.28 (d, J=8.6 Hz, 2H), 7.57 (d, 1H), 7.62 (t, 1H), 8.69 (d, J=7.3 Hz, 1H).

### Compound 2: 2-(1-(4-chlorophenyl)cyclopropyl)-8-ethyl-3-hydroxyquinoline-4-carboxylic acid

Following the procedure described for the preparation of Compound 1, intermediate 63 (0.38 g, 2.2 mmol) was reacted with intermediate 55 (0.71 g, 2.8 mmol). Acidification of the purified product did not give a solid precipitate and the aqueous acetonitrile mixture was extracted with ethyl acetate. The combined ethyl acetate layers were concentrated and lyophilized to give a fluffy, bright yellow solid (Compound 2, 140 mg, 18% yield). ¹H NMR (400 MHz, DMSO-d₆) δ 1.32 (t, J=7.5 Hz, 3H), 1.36-1.42 (m, 2H), 1.50-1.61 (m, 2H), 3.23 (q, J=7.5 Hz, 2H), 7.19 (d, 2H), 7.29 (d, J=8.6 Hz, 2H), 7.45 (d, 1H), 7.51 (t, 1H), 8.35 (d, J=8.3 Hz, 1H).

### Compound 3: 8-sec-butyl-2-(1-(4-chlorophenyl)cyclopropyl)-3-hydroxyquinoline-4-carboxylic acid

Compound 3 was prepared following the procedure described for the preparation of Compound 2, using as starting material intermediate 65 (0.44 g, 2.2 mmol) and intermediate 55 (0.71 g, 2.8 mmol), as a fluffy, bright yellow solid (81 mg, 9.5% yield). ¹H NMR (400 MHz, DMSO-d₆) δ 0.82 (t, J=7.3 Hz, 3H), 1.32 (d, J=7.1 Hz, 3H), 1.35-1.44 (m, 2H), 1.46-1.60 (m, 2H), 1.63-1.85 (m, 2H), 4.10 (q, 1H), 7.19 (d, J=8.6 Hz, 2H), 7.29 (d, J=8.6 Hz, 2H), 7.44 (d, J=7.1 Hz, 1H), 7.55 (t, 1H), 8.32 (d, J=8.3 Hz, 1H).

### Compound 4: 8-tert-butyl-2-(1-(4-chlorophenyl)cyclopropyl)-3-hydroxyquinoline-4-carboxylic acid

Compound 4 was prepared following the procedure described for the preparation of Compound 2, using as starting materials intermediate 67 (0.44 g, 2.2 mmol) and intermediate 55 (0.71 g, 2.8 mmol), as a fluffy, light brown solid (59 mg, 3.4% yield). ¹H NMR (400 MHz, DMSO-d₆) δ 1.36-1.42 (m, 2H), 1.48-1.54 (m, 2H), 1.65 (s, 9H), 7.22 (d, 2H), 7.30 (d, 2H), 7.45-7.54 (m, 2H), 8.26 (dd, J=7.5, 2.2 Hz, 1H).

### Compound 5: 8-chloro-2-(1-(4-chlorophenyl)cyclopropyl)-3-hydroxyquinoline-4-carboxylic acid

Compound 5 was prepared following the procedure described for the preparation of Compound 2, using as starting materials 7-chloroindoline-2,3-dione (Advanced Synthesis, 0.39 g, 2.2 mmol) and intermediate 55 (0.71 g, 2.8 mmol), as a fluffy, bright yellow solid (93 mg, 11% yield). ¹H NMR (400 MHz, DMSO-d₆) δ 1.32-1.38 (m, 2H), 1.51-1.58 (m, 2H), 7.15 (d, J=8.6 Hz, 2H), 7.27 (d, J=8.3 Hz, 2H), 7.47 (t, 1H), 7.64 (d, J=7.6 Hz, 1H), 8.87 (d, J=8.3 Hz, 1H).

### Compound 6: 2-(1-(4-chlorophenyl)cyclopropyl)-3-hydroxy-8-phenylquinoline-4-carboxylic acid

Compound 6 was prepared following the procedure described for the preparation of Compound 1, using as starting materials intermediate 11 (7-phenylindoline-2,3-dione, 0.48 g, 2.2 mmol) and intermediate 55 (0.71 g, 2.8 mmol). The cooled reaction mixture was filtered to remove Pd black left over from the Suzuki coupling step, acidified with 1 M hydrochloric acid, and extracted with ethyl acetate. The crude product was purified by preparative HPLC as described above and acidification of an aqueous acetonitrile solution of the purified triethylammonium salt gave a bright yellow powder, which was collected by filtration and dried under vacuum to give Compound 5 (249 mg, 28% yield). ¹H NMR (400 MHz, DMSO-d₆) δ 1.26-1.31 (m, 2H), 1.46-1.52 (m, 2H), 7.06 (d, J=8.6 Hz, 2H), 7.24 (d, J=8.6 Hz, 2H), 7.39 (t, J=7.3 Hz, 1H), 7.48 (t, J=7.6 Hz, 2H), 7.58-7.70 (m, 4H), 8.54 (dd, J=8.6, 1.3 Hz, 1H).

### Compound 7: 2-(1-(4-chlorophenyl)cyclopropyl)-8-fluoro-3-hydroxyquinoline-4-carboxylic acid

Compound 7 was prepared following the procedure described for the preparation of Compound 1, using as starting materials intermediate 69 (495 mg, 3.00 mmol) and intermediate 55 (0.99 g, 3.9 mmol). The cooled reaction mixture was acidified with 2 M hydrochloric acid and extracted with ethyl acetate. The crude product was purified by preparative HPLC (water/acetonitrile with 0.1% triethylamine). Fractions containing Compound 7 were combined, concentrated to remove acetonitrile, chilled in an ice-water bath, and acidified with concentrated hydrochloric acid. White precipitate was collected by filtration, washed with water, and dried under vacuum to give Compound 7 (300 mg, 28% yield). ¹H NMR (400 MHz, DMSO-d₆) δ 1.35-1.45 (m, 2H), 1.45-1.55 (m, 2H), 7.16 (dt, J=9.0, 2.8 Hz, 2H), 7.28 (dt, J=9.1, 2.5 Hz, 2H), 7.39 (ddd, 1H), 7.57 (dt, J=8.2, 5.6 Hz, 1H), 8.39 (d, J=8.8 Hz, 1H).

### Compound 8: 2-(1-(4-chlorophenyl)cyclopropyl)-3-hydroxyquinoline-4-carboxylic acid

Following the procedure described for the preparation of Compound 1, indoline-2,3-dione (Aldrich, 441 mg, 3.00 mmol) was reacted with intermediate 55 (0.99 g, 3.9 mmol). Acidification of the cooled reaction mixture with concentrated hydrochloric acid produced a bright yellow precipitate, which was collected by filtration, washed with water, dried under vacuum, and recrystallized from acetonitrile/ethanol. Compound 8 was obtained as a fine, bright yellow crystalline material (272 mg, 27% yield). ¹H NMR (400 MHz, DMSO-d₆) δ 1.37-1.44 (m, 2H), 1.50-1.57 (m, 2H), 7.19 (dt, 2H), 7.29 (dt, J=9.1, 2.7 Hz, 2H), 7.56-7.67 (m, 2H), 7.99-8.04 (m, 1H), 8.72 (s, 1H).

### Compound 9: 8-bromo-2-(1-(4-chlorophenyl)cyclopropyl)-3-hydroxyquinoline-4-carboxylic acid

Following the procedure described for the preparation of Compound 1, intermediate 71 (678 mg, 3.00 mmol) was reacted with intermediate 55 (0.99 g, 3.9 mmol). The cooled reaction mixture was acidified to pH 4 with glacial acetic acid and extracted with ethyl acetate. The combined ethyl acetate layers were concentrated to give the crude product, where was purified by preparative HPLC (water/acetonitrile with 0.1% triethylamine). Fractions containing Compound 9 were combined, concentrated to remove acetonitrile, acidified with concentrated hydrochloric acid, and extracted with ethyl acetate. The combined ethyl acetate layers were lyophilized to give a bright yellow powder (Compound 9, 303 mg, 24% yield). ¹H NMR (400 MHz, DMSO-d₆) δ 1.36-1.47 (m, 2H), 1.54-1.65 (m, 2H), 7.17 (dt, J=9.0, 2.8 Hz, 2H), 7.29 (dt, J=9.1, 2.5 Hz, 2H), 7.49 (dd, J=8.6, 7.3 Hz, 1H), 7.95 (dd, J=7.5, 1.1 Hz, 1H), 8.58 (dd, J=8.6, 1.3 Hz, 1H).

### Compound 10: 2-(1-(4-chlorophenyl)cyclopropyl)-3-hydroxy-6,8-dimethylquinoline-4-carboxylic acid

Following the procedure described for the preparation of Compound 1,5,7-dimethylindoline-2,3-dione (Lancaster, 526 mg, 3.00 mmol) was reacted with intermediate 55 (0.99 g, 3.9 mmol). Acidification of the cooled reaction mixture with concentrated hydrochloric acid gave a bright yellow precipitate, which was collected by filtration, dried under vacuum, and purified by preparative HPLC (water/acetonitrile with 0.1% triethylamine). Fractions containing Compound 10 were concentrated to remove acetonitrile, acidified with concentrated hydrochloric acid, and extracted with ethyl acetate. The combined ethyl acetate layers were lyophilized to give a bright yellow powder (Compound 10, 298 mg, 27% yield). ¹H NMR (400 MHz, DMSO-d₆) δ 1.33-1.42 (m, 2H), 1.47-1.57 (m, 2H), 2.44 (s, 3H), 2.69 (s, 3H), 7.15 (dt, J=9.0, 2.8 Hz, 2H), 7.27 (dt, J=9.1, 2.77 Hz, 2H), 7.30 (s, 1H), 8.13 (s, 1H).

### Compound 11: 2-(1-(4-chlorophenyl)cyclopropyl)-3-hydroxy-8-methylquinoline-4-carboxylic acid

Compound 11 was prepared following the procedure described for the preparation of Compound 10, using as starting materials intermediate 73 (7-methylindoline-2,3-dione, 313 mg, 1.94 mmol) and intermediate 55 (0.64 g, 2.5 mmol), as a yellow powder (247 mg, 36% yield). ¹H NMR (400 MHz, DMSO-d₆) δ 1.36-1.43 (m, 2H), 1.50-1.58 (m, 2H), 2.74 (s, 3H), 7.16 (dt, J=9.0, 2.8 Hz, 2H), 7.28 (dt, J=9.0, 2.5 Hz, 2H), 7.43-7.51 (m, 2H), 8.36 (dd, J=8.2, 1.4 Hz, 1H).

### Compound 12: 2-(1-(4-chlorophenyl)cyclopropyl)-7-ethyl-3-hydroxyquinoline-4-carboxylic acid

Compound 12 was prepared following the procedure for Compound 10, using 4-ethylindoline-2,3-dione (Advanced Synthesis, 924 mg, 5.27 mmol) and intermediate 55 as starting materials, as a fluffy yellow solid (5.3 mg, 0.3% yield). ¹H NMR (400 MHz, DMSO-d₆) δ 1.28 (t, J=7.6 Hz, 3H), 1.35-1.42 (m, 2H), 1.48-1.54 (m, 2H), 2.78 (q, J=7.5 Hz, 2H), 7.20 (d, 2H), 7.28 (d, 2H), 7.49 (d, J=9.9 Hz, 1H), 7.80 (s, 1H), 8.73 (s, 1H).

### Compound 13: 2-(1-(4-chlorophenyl)cyclopropyl)-3-hydroxy-7-methylquinoline-4-carboxylic acid

Following the procedure described for the preparation of Compound 1, a mixture of intermediates 75 and 76 (3.34, 20.1 mmol) was reacted with intermediate 55 (6.80 g, 26.9 mmol). Acidification of the cooled reaction mixture with 1 M hydrochloric acid produced a bright yellow precipitate, which was collected by filtration, washed with water, dried under vacuum, and triturated with boiling acetonitrile/ethanol to give Compound 14 (1.64 g, 22% yield). ¹H NMR (400 MHz, DMSO-d₆) δ 1.36-1.42 (m, 2H), 1.49-1.54 (m, 2H), 2.48 (s, 3H), 7.16-7.23 (m, 2H), 7.26-7.32 (m, 2H), 7.46 (d, J=9.9 Hz, 1H), 7.80 (s, 1H), 8.69 (s, 1H).

### Compound 14: 8-ethyl-3-hydroxy-2-(1-phenylcyclopropyl)quinoline-4-carboxylic acid

Intermediate 63 (0.38 g, 2.2 mmol) in ethanol was treated with 10.0 N aqueous sodium hydroxide solution (9.0 eq.) and the mixture was heated at reflux temperature. To this solution was added a solution of intermediate 8 (0.6 g, 2.8 mmol) in ethanol over 60 minutes. The resulting mixture was allowed to stir at reflux temperature for an additional 3 hours. Upon cooling to room temperature, ethanol was removed under reduced pressure. The mixture was acidified to pH 1 with 1M HCl and poured into water. The crude solid obtained was purified by reverse-phase HPLC (water/acetonitrile/0.1% triethyl amine). Fractions containing Compound 14 were combined and lyophilized to give the desired product (0.146 g, 20% yield). ¹H NMR (400 MHz, DMSO-d₆) δ 1.32 (t, J=7.5 Hz, 3H), 1.34-1.41 (m, 2H), 1.46-1.54 (m, 2H), 3.23 (q, J=7.3 Hz, 2H), 7.09-7.29 (m, 5H), 7.39-7.55 (m, 2H), 8.37 (dd, J=8.5, 1.39 Hz, 1H).

### Compound 15: 8-sec-butyl-3-hydroxy-2-(1-phenylcyclopropyl)quinoline-4-carboxylic acid

Following the procedure described for the preparation of Compound 14, intermediate 65 (0.373 g, 1.8 mmol) was reacted with intermediate 8 (0.5 g, 2.3 mmol) to give Compound 15 (0.116 g, 18% yield). ¹H NMR (400 MHz, DMSO-d₆) δ 0.82 (t, J=7.3 Hz, 3H), 1.32 (d, J=6.8 Hz, 3H), 1.34-1.41 (m, 2H), 1.42-1.55 (m, 2H), 1.61-1.90 (m, 2H), 3.06-3.13 (m, 1H), 7.07-7.27 (m, 5H), 7.41 (d, J=7.1 Hz, 1H), 7.47-7.60 (m, 1H), 8.39 (d, J=8.3 Hz, 1H).

### Compound 16: 7-chloro-2-(1-(4-chlorophenyl)cyclopropyl)-3-hydroxyquinoline-4-carboxylic acid

Following the procedure described for the preparation of Compound 14, 6-chloroindoline-2,3-dione (0.182 g, 1 mmol) was reacted with intermediate 55 (0.316 g, 1.25 mmol) to yield Compound 16 (0.06 g, 19% yield). ¹H NMR (400 MHz, DMSO-d₆) δ 1.28-1.44 (m, 2H), 1.44-1.58 (m, 2H), 7.17 (d, J=8.6 Hz, 2H), 7.28 (d, J=8.3 Hz, 2H), 7.62 (dd, J=9.2, 2.2 Hz, 1H), 8.01 (d, J=2.3 Hz, 1H), 8.75 (d, J=9.4 Hz, 1H).

### Compound 17: 2-(1-(4-chlorophenyl)cyclopropyl)-6-fluoro-3-hydroxyquinoline-4-carboxylic acid

Following the procedure described for the preparation of Compound 14, 5-fluoroindoline-2,3-dione (0.165 g, 1 mmol) was reacted with intermediate 55 (0.316 g, 1.25 mmol) to yield Compound 17 (0.1 g, 28% yield). ¹H NMR (400 MHz, DMSO-d₆) δ 1.30-1.41 (m, 2H), 1.43-1.55 (m, 2H), 7.14-7.21 (m, 2H), 7.23-7.31 (m, 2H), 7.37-7.50 (m, 1H), 8.02 (dd, J=9.1, 6.1 Hz, 1H), 8.58 (d, J=12.6 Hz, 1H).

### Compound 18: 6-bromo-2-(1-(4-chlorophenyl)cyclopropyl)-3-hydroxyquinoline-4-carboxylic acid

Following the procedure described for the preparation of Compound 14, 5-bromoindoline-2,3-dione (0.226 g, 1 mmol) was reacted with intermediate 55 (0.316 g, 1.25 mmol) to yield Compound 18 (0.12 g, 29% yield). ¹H NMR (400 MHz, DMSO-d₆) δ 1.29-1.38 (m, 2H), 1.43-1.54 (m, 2H), 7.11-7.22 (m, 2H), 7.22-7.32 (m, 2H), 7.60 (dd, J=8.84, 2.27 Hz, 1H), 7.86 (d, J=8.84 Hz, 1H), 9.17 (d, J=2.02 Hz, 1H)

### Compound 19: 2-(1-(4-chlorophenyl)cyclopropyl)-3-hydroxy-6-methylquinoline-4-carboxylic acid

Following the procedure described for the preparation of Compound 14, 5-methylindoline-2,3-dione (0.161 g, 1 mmol) was reacted with intermediate 55 (0.316 g, 1.25 mmol) to yield Compound 19 (0.10 g, 28% yield). ¹H NMR (400 MHz, DMSO-d₆) δ 1.34-1.44 (m, 2H), 1.46-1.61 (m, 2H), 2.50 (s, 3H), 7.19 (d, J=8.3 Hz, 2H), 7.24-7.34 (m, 2H), 7.34-7.48 (m, 1H), 7.89 (d, J=8.3 Hz, 1H), 8.57 (br s, 1H).

### Compound 20: 2-(1-(4-chlorophenyl)cyclopropyl)-3-hydroxy-6-methoxyquinoline-4-carboxylic acid

Following the procedure described for the preparation of Compound 14, 5-methoxyindoline-2,3-dione (0.177 g, 1 mmol) was reacted with intermediate 55 (0.316 g, 1.25 mmol) to yield Compound 20 (0.07 g, 19% yield). ¹H NMR (400 MHz, DMSO-d₆) δ 1.39 (s, 2H), 1.43-1.57 (m, 2H), 3.87 (s, 3H), 7.06-7.37 (m, 5H), 7.92 (d, J=9.1 Hz, 1H), 8.35 (br s, 1H).

### Compound 21: 2-(1-(4-chlorophenyl)cyclopropyl)-3-hydroxy-6-(trifluoromethoxy)quinoline-4-carboxylic acid

Following the procedure described for the preparation of Compound 14, 5-(trifluoromethoxy)indoline-2,3-dione (0.231 g, 1 mmol) was reacted with intermediate 55 (0.316 g, 1.25 mmol) to yield Compound 22 (0.148 g, 35% yield). ¹H NMR (400 MHz, DMSO-d₆) δ 1.28-1.41 (m, 2H), 1.41-1.60 (m, 2H), 7.09-7.22 (m, 2H), 7.22-7.34 (m, 2H), 7.43 (d, J=11.4 Hz, 1H), 8.02 (d, J=9.1 Hz, 1H), 8.99 (s, 1H).

### Compound 22: 6-chloro-2-(1-(4-chlorophenyl)cyclopropyl)-3-hydroxyquinoline-4-carboxylic acid

Following the procedure described for the preparation of Compound 14, 5-chloroindoline-2,3-dione (0.182 g, 1 mmol) was reacted intermediate 55 (0.316 g, 1.25 mmol) to yield Compound 22 (0.101 g, 27% yield). ¹H NMR (400 MHz, DMSO-d₆) δ 1.30-1.43 (m, 2H), 1.43-1.58 (m, 2H), 7.07-7.22 (m, 2H), 7.23-7.37 (m, 2H), 7.57 (dd, J=8.8, 2.3 Hz, 1H), 7.98 (d, J=8.8 Hz, 1H), 8.85 (d, J=1.8 Hz, 1H).

### Compound 23: 2-(1-(4-chlorophenyl)cyclopropyl)-3,6-dihydroxyquinoline-4-carboxylic acid

Following the procedure described for the preparation of Compound 14, 5-hydroxyindoline-2,3-dione (0.163 g, 1 mmol) was reacted with intermediate 55 (0.316 g, 1.25 mmol) to yield Compound 23 (0.09 g, 25% yield). ¹H NMR (400 MHz, DMSO-d₆) δ 1.40 (s, 2H), 1.47-1.56 (m, 2H), 7.13 (dd, J=9.0, 2.7 Hz, 1H), 7.16-7.25 (m, 2H), 7.25-7.33 (m, 2H), 7.84-7.92 (m, 1H), 8.24 (br s, 1H), 10.20 (br s, 1H).

### Compound 24: 2-(1-(4-chlorophenyl)cyclopropyl)-3-hydroxy-6-(trifluoromethyl)quinoline-4-carboxylic acid

Following the procedure described for the preparation of Compound 14, 5-(trifluoromethyl)indoline-2,3-dione (0.215 g, 1 mmol) was reacted with intermediate 55 (0.316 g, 1.25 mmol) to yield Compound 24 (0.041 g, 10% yield). ¹H NMR (400 MHz, DMSO-d₆) δ 1.28-1.45 (m, 2H), 1.47-1.67 (m, 2H), 7.10-7.23 (m, 2H), 7.24-7.38 (m, 2H), 7.79 (s, 1H), 8.16 (d, J=8.8 Hz, 1H), 9.26 (s, 1H).

### Compound 25: 2-(1-(4-chlorophenyl)cyclopropyl)-3-hydroxy-6-isopropylquinoline-4-carboxylic acid

Following the procedure described for the preparation of Compound 14, Intermediate 105 (5-isopropylindoline-2,3-dione, 0.189 g, 1 mmol) was reacted with intermediate 55 (0.316 g, 1.25 mmol) to yield Compound 25 (0.06 g, 16% yield). ¹H NMR (400 MHz, DMSO-d₆) δ 1.28 (d, J=6.8 Hz, 6H), 1.40 (s, 2H), 1.46-1.58 (m, 2H), 2.86-3.17 (m, 1H), 7.11-7.22 (m, 2H), 7.22-7.32 (m, 2H), 7.53 (dd, J=8.6, 1.77 Hz, 1H), 7.95 (d, J=8.6 Hz, 1H), 8.62 (br s, 1H).

### Compound 26: 7-chloro-3-hydroxy-2-(1-Phenylcyclopropyl)quinoline-4-carboxylic acid

Following the procedure described for the preparation of Compound 14, 6-chloroindoline-2,3-dione (0.156 g, 0.86 mmol) was reacted with intermediate 8 (0.234 g, 1.1 mmol) to yield Compound 26 (0.07 g, 20% yield). ¹H NMR (400 MHz, DMSO-d₆) δ 1.31-1.43 (m, 2H), 1.44-1.55 (m, 2H), 6.99-7.32 (m, 5H), 7.61 (dd, J=9.5, 2.2 Hz, 1H), 8.01 (d, J=2.5 Hz, 1H), 8.78 (d, J=9.4 Hz, 1H).

### Compound 27: 6-ethyl-3-hydroxy-2-(1-phenylcyclopropyl)quinoline-4-carboxylic acid

Following the procedure described for the preparation of Compound 14, 5-ethylindoline-2,3-dione (0.1 g, 0.57 mmol) was reacted intermediate 8 (0.156 g, 0.72 mmol) to yield Compound 27 (0.066 g, 18% yield). ¹H NMR (400 MHz, DMSO-d₆) δ 1.26 (t, J=7.6 Hz, 3H), 1.50 (s, 2H), 2.66-2.93 (m, 2H), 7.06-7.33 (m, 5H), 7.48 (dd, J=8.6, 1.52 Hz, 1H), 7.94 (d, J=8.6 Hz, 1H), 8.58 (s, 1H).

### Compound 28: 7-ethyl-3-hydroxy-2-(1-phenylcyclopropyl)quinoline-4-carboxylic acid

Following the procedure described for the preparation of Compound 14, 6-ethylindoline-2,3-dione (0.175 g, 1 mmol) was reacted with intermediate 8 (0.273 g, 1.25 mmol) to yield Compound 28 (0.07 g, 21% yield). ¹H NMR (400 MHz, DMSO-d₆) δ 1.28 (t, J=7.5 Hz, 3H), 1.35-1.44 (m, 2H), 1.47-1.53 (m, 2H), 2.79 (q, J=7.5 Hz, 2H), 7.07-7.31 (m, 5H), 7.50 (d, J=1.0 Hz, 1H), 7.83 (s, 1H), 8.69 (s, 1H).

### Compound 29: 3-hydroxy-2-(1-phenylcyclopropyl)-6-(trifluoromethoxy)quinoline-4-carboxylic acid

Following the procedure described for the preparation of Compound 14, 5-(trifluoromethoxy)indoline-2,3-dione (0.231 g, 1 mmol) was reacted with intermediate 8 (0.273 g, 1.25 mmol) to yield Compound 29 (0.11 g, 26% yield). ¹H NMR (400 MHz, DMSO-d₆) δ 1.27-1.43 (m, 2H), 1.42-1.54 (m, 2H), 7.01-7.31 (m, 5H), 7.46 (dd, J=9.1, 2.1 Hz, 1H), 8.05 (d, J=9.1 Hz, 1H), 8.95 (s, 1H).

### Compound 30: 6-chloro-3-hydroxy-2-(1-phenylcyclopropyl)quinoline-4-carboxylic acid

Following the procedure described for the preparation of Compound 14, 5-chloroindoline-2,3-dione (0.182 g, 1 mmol) was reacted with intermediate 8 (0.273 g, 1.25 mmol) to yield Compound 30 (0.09 g, 27% yield). ¹H NMR (400 MHz, DMSO-d₆) δ 1.25-1.44 (m, 2H), 1.43-1.58 (m, 2H), 6.98-7.32 (m, 5H), 7.57 (dd, J=8.8, 2.3 Hz, 1H), 8.00 (d, J=8.8 Hz, 1H), 8.86 (s, 1H).

### Compound 31: 3-hydroxy-8-methyl-2-(1-phenylcyclopropyl)quinoline-4-carboxylic acid

Following the procedure described for the preparation of Compound 14, intermediate 73 (7-methylindoline-2,3-dione, 0.161 g, 1 mmol) was reacted with intermediate 8 (0.273 g, 1.25 mmol) to yield Compound 31 (0.064 g, 20% yield). ¹H NMR (400 MHz, MeOD) δ 1.32-1.37 (m, 2H), 1.49-1.61 (m, 2H), 2.78 (s, 3H), 7.04-7.16 (m, 1H), 7.15-7.31 (m, 4H), 7.31-7.48 (m, 2H), 8.74 (dd, J=7.6, 2.3 Hz, 1H).

### Compound 32: 3-hydroxy-2-(1-phenylcyclopropyl)-6-(trifluoromethyl)quinoline-4-carboxylic acid

Following the procedure described for the preparation of Compound 14, 5-(trifluoromethyl)indoline-2,3-dione (0.215 g, 1 mmol) was reacted with intermediate 8 (0.273 g, 1.25 mmol) to yield Compound 32 (0.041 g, 11% yield). ¹H NMR (400 MHz, DMSO-d₆) δ 1.25-1.36 (m, 2H), 1.38-1.53 (m, 2H), 6.94-7.32 (m, 4H), 7.59 (dd, J=8.6, 2.0 Hz, 1H), 8.02 (d, J=8.3 Hz, 1H), 8.87 (br s, 1H), 9.72 (s, 1H).

### Compound 33: 3-hydroxy-6-methyl-2-(1-phenylcyclopropyl)quinoline-4-carboxylic acid

Following the procedure described for the preparation of Compound 14, 5-methylindoline-2,3-dione (0.161 g, 1 mmol) was reacted with intermediate 8 (0.273 g, 1.25 mmol) to yield Compound 33 (0.13 g, 40% yield). ¹H NMR (400 MHz, DMSO-d₆) δ 1.39-1.42 (m, 2H), 1.48-1.52 (m, 2H), 2.50 (s, 3H), 7.07-7.36 (m, 5H), 7.44 (dd, J=8.6, 1.8 Hz, 1H), 7.93 (d, J=8.1 Hz, 1H), 8.60 (s, 1H).

### Compound 34: 3-hydroxy-2-(1-phenylcyclopropyl)-8-(trifluoromethyl)quinoline-4-carboxylic acid

Following the procedure described for the preparation of Compound 14, intermediate 6 (7-trifluoromethyl-1H-indole-2,3-dione, 0.40 g, 1.86 mmol) was reacted with intermediate 8 (2-oxo-2-(1-phenylcyclopropyl)ethyl acetate, 0.45 g, 2.05 mmol) to yield Compound 34 as a light yellow solid (0.20 g, 29% yield). ¹H NMR (400 MHz, MeOH-D₄) δ 1.68 (dd, J=7.0, 4.7 Hz, 2H), 7.46 (dd, J=7.0, 4.7 Hz, 2H), 7.51-7.59 (m, 2H), 7.66 (dd, J=8.6, 7.3 Hz, 2H), 7.87 (dd, J=8.4, 1.5 Hz, 1H), 8.14 (d, J=9.0 Hz, 1H), 9.91 (d, J=9.0 Hz, 1H).

### Compound 35: 3-hydroxy-2-(1-phenylcyclopropyl)-8-(thiophen-3-yl)quinoline-4-carboxylic acid

Following the procedure described for the preparation of Compound 14, intermediate 54 (7-(thiophen-3-yl)indoline-2,3-dione, 0.30 g, 1.30 mmol) was reacted with intermediate 8 (2-oxo-2-(1-phenylcyclopropyl)ethyl acetate, 0.31 g, 1.40 mmol) to yield Compound 35 as a light yellow solid (0.12 g, 24% yield). ¹H NMR (400 MHz, MeOH-D₄) δ 1.52 (dd, J=7.1, 4.0 Hz, 2H), 1.73-1.78 (dd, J=7.1, 4.0 Hz, 2H), 7.25-7.34 (m, 1H), 7.35-7.43 (m, 1H), 7.43-7.50 (m, 1H), 7.62-7.72 (m, 2H), 7.83-7.87 (m, 2H), 7.88-7.93 (m, 2H), 8.13-8.18 (m, 1H), 9.41-9.47 (m, 1H).

### Compound 36: 2-(1-(4-chlorophenyl)cyclopropyl)-3-hydroxy-7,8,9,10-tetrahydrobenzo[h]quinoline-4-carboxylic acid

Following the procedure described for the preparation of Compound 14, intermediate 3 (0.16 g, 0.80 mmol) was reacted with intermediate 55 (2-(1-(4-chlorophenyl)cyclopropyl)-2-oxoethyl acetate, 0.22 g, 0.88 mmol) to yield Compound 36 as a yellow solid (33.3 mg, 10.6% yield). ¹H NMR (400 MHz, DMSO-D₆) 11.22-1.32 (m, 2H), 1.40-1.48 (m, 2H), 1.72-1.91 (m, 4H), 2.75-2.87 (m, 2H), 3.17-3.26 (m, 2H), 7.13-7.18 (m, 3H), 7.24 (d, J=8.1 Hz, 2H), 7.37-7.48 (m, 1H), 8.85-9.08 (m, 2H).

### Compound 37: 2-(1-(4-chlorophenyl)cyclopropyl)-3-hydroxy-8-(thiophen-3-yl)quinoline-4-carboxylic acid

Following the procedure described for the preparation of Compound 14, intermediate 54 (0.19 g, 0.83 mmol) was reacted with intermediate 55 (2-(1-(4-chlorophenyl)cyclopropyl)-2-oxoethyl acetate, 0.23 g, 0.91 mmol) to yield Compound 37 as a yellow solid (110 mg, 31.4% yield). ¹H NMR (400 MHz, CDCl₃) δ 1.62 (dd, J=6.8, 4.7 Hz, 2H), 2.38-2.65 (m, 2H), 7.16 (d, J=8.9 Hz, 2H), 7.22 (d, J=8.9 Hz, 2H), 7.37 (dd, J=5.1, 3.1 Hz, 1H), 7.49 (dd, J=8.6, 7.2 Hz, 1H), 7.66 (dd, J=7.2, 1.5 Hz, 1H), 7.70 (dd, J=5.1, 1.2 Hz, 1H), 7.97 (dd, J=3.1, 1.2 Hz, 1H), 9.27 (dd, J=8.6, 1.5 Hz, 1H).

### Compound 38: 2-(1-(4-chlorophenyl)cyclopropyl)-3-hydroxy-8-(trifluoromethyl)quinoline-4-carboxylic acid

Following the procedure described for the preparation of Compound 14, intermediate 6 (7-(trifluoromethyl)indoline-2,3-dione, 0.41 g, 1.91 mmol) was reacted with intermediate 55 (2-(1-(4-chlorophenyl)cyclopropyl)-2-oxoethyl acetate, 0.53 g, 2.10 mmol) to yield Compound 38 as a yellow solid (190 mg, 24.4% yield). ¹H NMR (400 MHz, MeOH-D₄) δ 1.58 (dd, J=7.5, 4.6 Hz, 2H), 1.82 (dd, J=7.5, 4.6 Hz, 2H), 7.44 (d, J=8.7 Hz, 2H), 7.53 (d, J=8.7 Hz, 2H); 7.87 (dd, J=8.7, 7.6 Hz, 1H), 8.14 (d, J=7.6 Hz, 1H), 9.29 (d, J=8.7 Hz, 1H).

### Compound 39: 2-(1-(4-chlorophenyl)cyclopropyl)-3-hydroxy-8-isopropylquinoline-4-carboxylic acid

Following the procedure described for the preparation of Compound 14, intermediate 5 (7-isopropylindoline-2,3-dione, 0.16 g, 0.83 mmol) was reacted with intermediate 55 (2-(1-(4-chlorophenyl)cyclopropyl)-2-oxoethyl acetate, 0.19 g, 0.91 mmol) to yield Compound 39 as a yellow solid (134 mg, 42.3% yield). ¹H NMR (400 MHz, CDCl₃) δ 1.33 (dd, J=6.9, 4.6 Hz, 2H), 1.37 (d, J=6.9 Hz, 6H), 1.60 (dd, J=6.9, 4.6 Hz, 2H), 4.37 (sept, J=6.9 Hz, 1H), 7.15 (d, J=8.6 Hz, 2H), 7.24 (d, J=8.6 Hz, 2H), 7.33 (dd, J=7.3, 1.2 Hz, 1H), 7.41 (dd, J=8.5, 7.3 Hz, 1H), 9.00 (dd, J=8.5 Hz, 1H).

### Compound 40: 2-(1-(4-chlorophenyl)cyclopropyl)-3-hydroxy-7,8-dimethylquinoline-4-carboxylic acid

Following the procedure described for the preparation of Compound 14, intermediate 4 (6,7-dimethylindoline-2,3-dione, 70 mg, 0.39 mmol) was reacted with intermediate 55 (2-(1-(4-chlorophenyl)cyclopropyl)-2-oxoethyl acetate, 108 mg, 0.43 mmol) to yield Compound 40 as a yellow solid (42.5 mg, 29.7% yield). ¹H NMR (400 MHz, CDCl₃) δ 1.34 (dd, J=7.2, 4.0 Hz, 2H), 1.62 (dd, J=7.2, 4.0 Hz, 2H), 2.43-2.47 (s, 3H), 2.74-2.78 (s, 3H), 7.15 (d, J=8.5 Hz, 2H), 7.20 (d, J=8.5 Hz, 2H), 7.30 (d, J=9.0 Hz, 1H), 8.97 (d, J=9.0 Hz, 1H).

### Compound 41: 2-(1-(4-chlorophenyl)cyclopropyl)-8-(1,1,1,3,3,3-hexafluoro-2-hydroxypropan-2-yl)-3-hydroxyquinoline-4-carboxylic acid

Following the procedure described for the preparation of Compound 14, intermediate 16 (7-(1,1,1,3,3,3-hexafluoro-2-hydroxypropan-2-yl)indoline-2,3-dione, 240 mg, 0.77 mmol) was reacted with intermediate 55 (2-(1-(4-chlorophenyl)cyclopropyl)-2-oxoethyl acetate, 212 mg, 0.85 mmol) to yield Compound 41 as a white solid (28.5 mg, 7.3% yield). ¹H NMR (400 MHz, MeOH-D₄) δ 1.67-1.74 (m, 4H), 7.49 (dd, J=9.7 Hz, 2H), 7.57 (d, J=9.7 Hz, 2H), 7.92 (dd, J=8.4, 8.4 Hz, 1H), 8.12 (d, J=8.4 Hz, 1H), 9.27 (d, J=8.4 Hz, 1H).

### Compound 42: 3-hydroxy-2-(1-phenylcyclopropyl)-7,8,9,10-tetrahydrobenzo[h]quinoline-4-carboxylic acid

Following the procedure described for the preparation of Compound 14, intermediate 3 (6,7,8,9-tetrahydro-1H-benzo[g]indole-2,3-dione, 1.34 g, 5.4 mmol, 1.0 eq.) was reacted with intermediate 8 (2-oxo-2-(1-phenylcyclopropyl)ethyl acetate, 1.51 g, 6.93 mmol, 1.3 eq.) in the presence of 10.0 N aqueous sodium hydroxide solution (5.0 mL, 48.6 mmol, 9.0 eq.). Compound 42 was obtained as a yellow powder (0.2799 g, 14% yield). ¹H NMR (400 MHz, DMSO-d₆) δ 1.29-1.39 (m, 2H), 1.44-1.56 (m, 2H), 1.74-1.91 (m, 4H), 2.84 (t, J=5.43 Hz, 2H), 3.26 (t, J=6.06 Hz, 2H), 7.10-7.17 (m, 3H), 7.18-7.23 (m, 2H), 7.28 (d, J=8.59 Hz, 1H), 8.36 (d, J=9.35 Hz, 1H).

### Compound 43: 3-hydroxy-7,8-dimethyl-2-(1-phenylcyclopropyl)quinoline-4-carboxylic acid

Following the procedure described for the preparation of Compound 14, intermediate 8 (2-oxo-2-(1-phenylcyclopropyl)ethyl acetate, 1.65 g, 7.4 mmol, 1.3 eq.) was reacted with intermediate 4 (6,7-dimethylindoline-2,3-dione, 1.0 g, 5.71 mmol, 1.0 eq.) in the presence of 10.0 N aqueous sodium hydroxide solution (5.1 mL, 51.4 mmol, 9.0 eq.). Compound 43 was obtained as a yellow powder (0.732 g, 39% yield). ¹H NMR (400 MHz, DMSO-d₆) δ 1.29-1.41 (m, 2H), 1.46-1.62 (m, 2H), 2.42 (s, 3H), 2.71 (s, 3H), 7.10-7.16 (m, 3H), 7.18-7.26 (m, 2H), 7.41 (d, J=8.59 Hz, 1H), 8.28 (d, J=8.84 Hz, 1H).

### Compound 44: 3-hydroxy-8-isopropyl-2-(1-phenylcyclopropyl)quinoline-4-carboxylic acid

Following the procedure described for the preparation of Compound 14, intermediate 8 (2-oxo-2-(1-phenylcyclopropyl)ethyl acetate, 0.80 g, 3.6 mmol, 0.7 eq.) was reacted with intermediate 5 (7-isopropylindoline-2,3-dione, 1.0 g, 5.29 mmol, 1.0 eq.) in the presence of 10.0 N aqueous sodium hydroxide solution (4.8 mL, 47.6 mmol, 9.0 eq.). Compound 44 was obtained as a yellow powder (0.724 g, 40% yield). ¹H NMR (400 MHz, DMSO-d₆) δ 1.35 (d, J=7.07 Hz, 6H), 1.36-1.40 (m, 2H), 1.43-1.53 (m, 2H), 3.53-5.07 (h, J=8.59 Hz, 1H), 7.00-7.30 (m, 5H), 7.40-7.48 (m, 1H), 7.48-7.59 (m, 1H), 8.37 (d, J=8.59 Hz, 1H).

### Compound 45: 3-hydroxy-8-phenyl-2-(1-Phenylcyclopropyl)quinoline-4-carboxylic acid

Following the procedure described for the preparation of Compound 14, intermediate 8 (2-oxo-2-(1-phenylcyclopropyl)ethyl acetate, 0.133 g, 0.61 mmol, 1.3 eq.) was reacted with intermediate 11 (7-phenylindoline-2,3-dione, 0.105 g, 0.47 mmol, 1.0 eq.) in the presence of 10.0 N aqueous sodium hydroxide solution (0.47 mL, 4.2 mmol, 9.0 eq.). Compound 45 was obtained as a yellow powder (0.032 g, 18% yield). ¹H NMR (400 MHz, DMSO-d₆) δ 1.22-1.32 (m, 2H), 1.37-1.50 (m, 2H), 6.98-7.06 (m, 1H), 7.07-7.13 (m, 1H), 7.14-7.24 (m, 2H), 7.33-7.42 (m, 1H), 7.48 (t, J=7.45 Hz, 2H), 7.53-7.59 (m, 1H), 7.60-7.70 (m, 4H), 8.64 (d, J=7.83 Hz, 1H).

### Compound 46: 3-hydroxy-2-(1-phenylcyclopropyl)-8-(trifluoromethoxy)quinoline-4-carboxylic acid

Following the procedure described for the preparation of Compound 14, intermediate 8 (2-oxo-2-(1-phenylcyclopropyl)ethyl acetate, 0.368 g, 1.69 mmol, 1.3 eq.) was reacted with intermediate 13 (7-(trifluoromethoxy)indoline-2,3-dione, 0.300 g, 1.30 mmol, 1.0 eq.) in the presence of 10.0 N aqueous sodium hydroxide solution (1.17 mL, 11.68 mmol, 9.0 eq.). Compound 46 was obtained as a yellow powder (0.076 g, 15% yield). ¹H NMR (400 MHz, DMSO-d₆) δ 1.30-1.39 (m, 2H), 1.42-1.53 (m, 2H), 6.99-7.18 (m, 3H), 7.18-7.27 (m, 2H), 7.46-7.73 (m, 2H), 8.74 (d, J=7.58 Hz, 1H).

### Compound 47: 8-chloro-3-hydroxy-2-(1-phenylcyclopropyl)quinoline-4-carboxylic acid

Following the procedure described for the preparation of Compound 14, intermediate 8 (2-oxo-2-(1-phenylcyclopropyl)ethyl acetate, 0.780 g, 3.58 mmol, 1.3 eq.) was reacted with 7-chloroindoline-2,3-dione (0.500 g, 2.75 mmol, 1.0 eq.) in the presence 10.0 N aqueous sodium hydroxide solution (2.48 mL, 24.78 mmol, 9.0 eq.). Compound 47 was obtained as a yellow powder (0.308 g, 33% yield). ¹H NMR (400 MHz, DMSO-d₆) δ 1.33-1.40 (m, 2H), 1.48-1.59 (m, 2H), 6.88-7.36 (m, 5H), 7.53 (t, J=8.59 Hz, 1H), 7.72 (dd, J=7.45, 1.14 Hz, 1H), 8.64 (d, J=8.59 Hz, 1H).

### Compound 48: 6-(1,1,1,3,3,3-hexafluoro-2-hydroxypropan-2-yl)-3-hydroxy-2-(1-phenylcyclopropyl)quinoline-4-carboxylic acid

Following the procedure described for the preparation of Compound 14, intermediate 15 (5-(1,1,1,3,3,3-hexafluoro-2-hydroxypropan-2-yl)indoline-2,3-dione, 0.50 g, 1.6 mmol, 1.0 eq.) was reacted with intermediate 8 (2-oxo-2-(1-phenylcyclopropyl)ethyl acetate, 0.383 g, 1.76 mmol, 1.1 eq.) in the presence of 10.0 N aqueous sodium hydroxide solution (1.4 mL, 14.4 mmol, 9.0 eq.). Compound 48 was obtained as a yellow powder (0.103 g, 14% yield). ¹H NMR (400 MHz, DMSO-d₆) δ 1.35-1.42 (m, 2H), 1.46-1.54 (m, 2H), 7.01-7.30 (m, 5H), 7.73-7.86 (m, 1H), 8.10 (d, J=8.84 Hz, 1H), 8.91 (s, 1H), 9.34 (s, 1H).

### Compound 49: 8-(1,1,1,3,3,3-hexafluoro-2-hydroxypropan-2-yl)-3-hydroxy-2-(1-phenylcyclopropyl)quinoline-4-carboxylic acid

Following the procedure described for the preparation of Compound 14, intermediate 16 (7-(1,1,1,3,3,3-hexafluoro-2-hydroxypropan-2-yl)indoline-2,3-dione, 5.64 g, 18.02 mmol, 1.0 eq.) was reacted intermediate 8 (2-oxo-2-(1-phenylcyclopropyl)ethyl acetate, 5.11 g, 23.42 mmol, 1.3 eq.) in the presence of 10.0 N aqueous sodium hydroxide solution (16.22 mL, 162.0 mmol, 9.0 eq.). Compound 49 was obtained as a yellow powder (2.52 g, 30% yield). ¹H NMR (400 MHz, DMSO-d₆) δ 1.33-1.40 (m, 2H), 1.40-1.49 (m, 2H), 7.13-7.30 (m, 5H), 7.65-7.71 (m, 1H), 7.72-7.79 (m, 1H), 9.06 (d, J=8.34 Hz, 1H).

### Compound 50: 3-hydroxy-2-(1-(4-methoxyphenyl)cyclopropyl)-8-(trifluoromethyl)quinoline-4-carboxylic acid

Following the procedure described for the preparation of Compound 14, intermediate 6 (7-(trifluoromethyl)indoline-2,3-dione, 0.100 g, 0.45 mmol, 1.0 eq.) was reacted with intermediate 18 (2-(1-(4-methoxyphenyl)cyclopropyl)-2-oxoethyl acetate, 0.144 g, 0.59 mmol, 1.3 eq.) in the presence of 10.0 N aqueous sodium hydroxide solution (0.5 mL, 5.4 mmol, 9.0 eq.). Compound 50 was obtained as a yellow powder (0.041 g, 17% yield). ¹H NMR (400 MHz, DMSO-d₆) δ 1.18-1.32 (m, 2H), 1.37-1.50 (m, 2H), 3.68 (s, 3H), 6.80 (d, J=9.09 Hz, 2H), 7.17 (d, J=8.84 Hz, 2H), 7.66 (dd, J=8.59, 6.82 Hz, 2H), 7.91 (d, J=6.82 Hz, 1H), 9.00 (d, J=8.59 Hz, 1H).

### Compound 51: 3-hydroxy-2-(1-(4-methoxyphenyl)cyclopropyl)-7,8,9,10-tetrahydrobenzo[h]quinoline-4-carboxylic acid

Following the procedure described for the preparation of Compound 14, intermediate 18 (2-(1-(4-methoxyphenyl)cyclopropyl)-2-oxoethyl acetate, 0.500 g, 2.04 mmol, 1.3 eq.) was reacted with intermediate 3 (6,7,8,9-tetrahydro-1H-benzo[g]indole-2,3-dione, 0.396 g, 1.57 mmol, 1.0 eq.) in the presence of 10.0 N aqueous sodium hydroxide (1.4 mL, 14.1 mmol, 9.0 eq.). Compound 51 was obtained as a yellow powder (0.057 g, 9.2% yield). ¹H NMR (400 MHz, DMSO-d₆) δ 1.19-1.30 (m, 2H), 1.39-1.47 (m, 4H), 1.75-1.96 (m, 4H), 2.84 (t, J=5.94 Hz, 2H), 3.27 (t, J=6.06 Hz, 2H), 3.68 (s, 3H), 6.78 (d, J=8.59 Hz, 2H), 7.14 (d, J=8.59 Hz, 2H), 7.28 (d, J=8.84 Hz, 1H), 8.27 (d, J=8.59 Hz, 1H).

### Compound 52: 3-hydroxy-8-(trifluoromethyl)-2-(1-(4-(trifluoromethyl)phenyl)cyclopropyl)quinoline-4-carboxylic acid

Following the procedure described for the preparation of Compound 14, intermediate 21 (2-hydroxy-1-(1-(4-(trifluoromethyl)phenyl)cyclopropyl)ethanone, 0.149 g, 0.6 mmol, 1.3 eq.) was reacted with intermediate 6 (7-(trifluoromethyl)indoline-2,3-dione, 0.101 g, 0.47 mmol, 1.0 eq.) in the presence of 10.0 N aqueous sodium hydroxide (0.4 mL, 4.23 mmol, 9.0 eq.). Compound 52 was obtained as a yellow powder (0.086 g, 33% yield). ¹H NMR (400 MHz, CDCl₃) δ 1.39-1.51 (m, 2H), 1.57-1.65 (m, 2H), 7.34 (d, J=8.08 Hz, 2H), 7.60 (d, J=8.34 Hz, 2H), 7.65-7.78 (m, 1H), 7.95 (d, J=7.33 Hz, 1H), 8.99 (d, J=8.59 Hz, 1H).

### Compound 53: 2-(1-(4-bromophenyl)cyclopropyl)-3-hydroxy-8-(trifluoromethyl)quinoline-4-carboxylic acid

Following the procedure described for the preparation of Compound 14, intermediate 25 (2-(1-(4-bromophenyl)cyclopropyl)-2-oxoethyl acetate, 0.091 g, 0.31 mmol, 1.3 eq.) was reacted with intermediate 6 (7-(trifluoromethyl)indoline-2,3-dione, 0.051 g, 0.24 mmol, 1.0 eq.) in the presence of 10.0 N aqueous sodium hydroxide solution (0.2 mL, 2.13 mmol, 9.0 eq.). Compound 53 was obtained as a yellow powder (0.033 g, 24% yield). ¹H NMR (400 MHz, DMSO-d₆) δ 1.28-1.38 (m, 2H), 1.45-1.54 (m, 2H), 7.12 (d, J=8.59 Hz, 2H), 7.41 (d, J=8.59 Hz, 2H), 7.63 (t, J=8.59 Hz, 1H), 7.86 (d, J=7.33 Hz, 1H), 9.18 (d, J=8.59 Hz, 1H).

### Compound 54: 2-(1-(3-chlorophenyl)cyclopropyl)-3-hydroxy-8-(trifluoromethyl)quinoline-4-carboxylic acid

Following the procedure described for the preparation of Compound 14, intermediate 28 (1-(1-(3-chlorophenyl)cyclopropyl)-2-hydroxyethanone, 0.255 g, 1.21 mmol, 1.3 eq.) was reacted with intermediate 6 (7-(trifluoromethyl)indoline-2,3-dione, 0.200 g, 0.93 mmol, 1.0 eq.) in the presence 10.0 N aqueous sodium hydroxide (0.84 mL, 8.4 mmol, 9.0 eq.). Compound 54 was obtained as a yellow powder (0.058 g, 15% yield). ¹H NMR (400 MHz, DMSO-d₆) δ 1.32-1.42 (m, 2H), 1.44-1.54 (m, 2H), 6.95-7.37 (m, 4H), 7.63 (t, J=7.96 Hz, 1H), 7.86 (d, J=7.33 Hz, 1H), 9.19 (d, J=8.59 Hz, 1H).

### Compound 55: 2-(1-(2-chlorophenyl)cyclopropyl)-3-hydroxy-8-(trifluoromethyl)quinoline-4-carboxylic acid

Following the procedure described for the preparation of Compound 14, intermediate 32 (2-(1-(2-chlorophenyl)cyclopropyl)-2-oxoethyl acetate, 0.306 g, 1.21 mmol, 1.3 eq.) was reacted with intermediate 6 (7-(trifluoromethyl)indoline-2,3-dione, 0.200 g, 0.93 mmol, 1.0 eq.) in the presence of 10.0 N aqueous sodium hydroxide (0.84 mL, 8.4 mmol, 9.0 eq.). Compound 55 was obtained as a yellow powder (0.029 g, 8% yield). ¹H NMR (400 MHz, DMSO-d₆) δ 1.14-1.23 (m, 2H), 1.77-1.89 (m, 2H), 7.14-7.22 (m, 1H), 7.23-7.33 (m, 2H), 7.38-7.50 (m, 1H), 7.61 (d, J=7.07 Hz, 1H), 7.72 (dd, J=7.71, 1.64 Hz, 1H), 9.61 (d, J=8.08 Hz, 1H).

### Compound 56: 3-hydroxy-2-(1-(4-(trifluoromethoxy)phenyl)cyclopropyl-8-(trifluoromethyl)quinoline-4-carboxylic acid

Following the procedure described for the preparation of Compound 14, intermediate 35 (2-hydroxy-1-(1-(4-(trifluoromethoxy)phenyl)cyclopropyl)ethanone, 0.315 g, 0.93 mmol, 1.3 eq.) was reacted with intermediate 6 (7-(trifluoromethyl)indoline-2,3-dione, 0.200 g, 0.93 mmol, 1.0 eq.) in the presence of 10.0 N aqueous sodium hydroxide (0.84 mL, 8.4 mmol, 9.0 eq.). Compound 56 was obtained as a yellow powder (0.142 g, 33% yield). ¹H NMR (400 MHz, DMSO-d₆) δ 1.33-1.44 (m, 2H), 1.47-1.58 (m, 2H), 7.19-7.25 (m, 2H), 7.25-7.31 (m, 2H), 7.60-7.76 (m, 1H), 7.92 (d, J=7.58 Hz, 1H), 9.03 (d, J=8.34 Hz, 1H).

### Compound 57: 3-hydroxy-8-(trifluoromethyl)-2-(1-(3-(trifluoromethyl)phenyl)cyclopropyl)quinoline-4-carboxylic acid

Following the procedure described for the preparation of Compound 14, intermediate 38 (2-hydroxy-1-(1-(3-(trifluoromethyl)phenyl)cyclopropyl)ethanone, 0.687 g, 2.82 mmol, 1.3 eq.) was reacted with intermediate 6 (7-(trifluoromethyl)indoline-2,3-dione, 0.466 g, 2.17 mmol, 1.0 eq.) in the presence of 10.0 N aqueous sodium hydroxide (1.9 mL, 19.5 mmol, 9.0 eq.). Compound 57 was obtained as a yellow powder (0.369 g, 30% yield). ¹H NMR (400 MHz, DMSO-d₆) δ 1.39-1.48 (m, 2H), 1.52-1.62 (m, 2H), 7.37-7.58 (m, 4H), 7.67 (t, J=8.34 Hz, 1H), 7.92 (d, J=7.07 Hz, 1H), 9.05 (d, J=8.34 Hz, 1H).

### Compound 58: 2-(1-(4-chlorophenyl)cyclobutyl)-3-hydroxy-8-(trifluoromethyl)quinoline-4-carboxylic acid

Following the procedure described for the preparation of Compound 14, intermediate 42 (2-(1-(4-chlorophenyl)cyclobutyl)-2-oxoethyl acetate, 0.476 g, 1.80 mmol, 1.3 eq.) was reacted with intermediate 6 (7-(trifluoromethyl)indoline-2,3-dione, 0.300 g, 1.40 mmol, 1.0 eq.) in the presence of 10.0 N sodium hydroxide (1.3 mL, 12.6 mmol, 9.0 eq.). Compound 58 was obtained as a white powder (0.293 g, 50% yield). ¹H NMR (400 MHz, DMSO-d₆) δ 1.73-2.06 (m, 2H), 2.55-2.78 (m, 2H), 2.95-3.25 (m, 2H), 7.35 (q, J=8.34 Hz, 4H), 7.69 (t, J=7.96 Hz, 1H), 7.97 (d, J=7.58 Hz, 1H), 8.92 (d, J=8.59 Hz, 1H).

### Compound 59: 3-hydroxy-2-(1-(thiophen-3-yl)cyclopropyl)-8-(trifluoromethyl)quinoline-4-carboxylic acid

Following the procedure described for the preparation of Compound 14, intermediate 45 (2-hydroxy-1-(1-(thiophen-3-yl)cyclopropyl)ethanone, 0.062 g, 0.34 mmol, 1.3 eq.) was reacted with intermediate 6 (7-(trifluoromethyl)indoline-2,3-dione, 0.056 g, 0.26 mmol, 1.0 eq.) in the presence of 10.0 N sodium hydroxide (0.24 mL, 2.36 mmol, 9.0 eq.). Compound 59 was obtained as a yellow powder (0.033 g, 26% yield). ¹H NMR (400 MHz, DMSO-d₆) δ 1.27-1.36 (m, 2H), 1.39-1.55 (m, 2H), 6.86 (dd, J=4.93, 1.39 Hz, 1H), 6.98 (dd, J=2.91, 1.39 Hz, 1H), 7.36 (dd, J=5.05, 3.03 Hz, 1H), 7.58-7.69 (m, 1H), 7.87 (d, J=7.07 Hz, 1H), 9.15 (d, J=8.59 Hz, 1H).

### Compound 60: 3-hydroxy-2-(1-(thiophen-2-yl)cyclopropyl)-8-(trifluoromethyl)quinoline-4-carboxylic acid

Following the procedure described for the preparation of Compound 14, intermediate 48 (2-hydroxy-1-(1-(thiophen-2-yl)cyclopropyl)ethanone, 0.387 g, 2.13 mmol, 1.3 eq.) was reacted with intermediate 6 (7-(trifluoromethyl)indoline-2,3-dione, 0.352 g, 1.64 mmol, 1.0 eq.) in the presence of 10.0 N sodium hydroxide (1.5 mL, 14.72 mmol, 9.0 eq.). Compound 60 was obtained as a yellow powder (0.251 g, 31% yield). ¹H NMR (400 MHz, DMSO-d₆) δ 1.33-1.43 (m, 2H), 1.49-1.63 (m, 2H), 6.82-6.85 (m, 1H), 6.86-6.89 (m, 1H), 7.24 (dd, J=5.05, 1.26 Hz, 1H), 7.61-7.74 (m, 1H), 7.92 (d, J=7.07 Hz, 1H), 9.01 (d, J=8.59 Hz, 1H).

### Compound 61: 2-(1-(4-fluorophenyl)cyclopropyl)-3-hydroxy-8-(trifluoromethyl)quinoline-4-carboxylic acid

Following the procedure described for the preparation of Compound 14, intermediate 51 (1-(1-(4-fluorophenyl)cyclopropyl)-2-hydroxyethanone, 0.590 g, 3.05 mmol, 1.3 eq.) was reacted with intermediate 6 (7-(trifluoromethyl)indoline-2,3-dione, 0.504 g, 2.34 mmol, 1.0 eq.) in the presence of 10.0 N sodium hydroxide (2.1 mL, 21.1 mmol, 9.0 eq.). Compound 61 was obtained as a yellow powder (0.132 g, 14% yield). ¹H NMR (400 MHz, DMSO-d₆) δ 1.24-1.35 (m, 2H), 1.40-1.51 (m, 2H), 6.93-7.12 (m, 2H), 7.17-7.34 (m, 2H), 7.62 (t, J=8.08 Hz, 1H), 7.86 (d, J=7.33 Hz, 1H), 9.17 (d, J=9.60 Hz, 1H).

### Compound 62: 2-(1-(4-fluorophenyl)cyclopropyl)-3-hydroxy-8-isopropylquinoline-4-carboxylic acid

Following the procedure described for the preparation of Compound 14, intermediate 51 (1-(1-(4-fluorophenyl)cyclopropyl)-2-hydroxyethanone, 0.223 g, 1.15 mmol, 1.3 eq.) was reacted with intermediate 5 (7-isopropylindoline-2,3-dione, 0.167 g, 0.88 mmol, 1.0 eq.) in the presence of 10.0 N sodium hydroxide (0.8 mL, 7.95 mmol, 9.0 eq.). Compound 62 was obtained as a yellow powder (0.126 g, 39% yield). ¹H NMR (400 MHz, DMSO-d₆) δ 1.28-1.39 (m, 8H), 1.43-1.53 (m, 2H), 4.15-4.34 (m, 1H), 6.94-7.12 (m, 2H), 7.19-7.29 (m, 2H), 7.41-7.47 (d, J=8.08 Hz, 1H), 7.48-7.56 (t, J=8.08 Hz, 1H), 8.40 (d, J=8.08 Hz, 1H).

### Compound 63: 3-hydroxy-8-(trifluoromethyl)-2-(1-(2-(trifluoromethyl)phenyl)cyclopropyl)quinoline-4-carboxylic acid

Following the procedure described for the preparation of Compound 14, intermediate 6 (7-(trifluoromethyl)indoline-2,3-dione, 0.136 g, 0.63 mmol, 1.0 eq.) was reacted with intermediate 104 (2-oxo-2-(1-(2-(trifluoromethyl)phenyl)cyclopropyl)ethyl acetate, 0.235 g, 0.82 mmol, 1.3 eq.) in the presence of 10.0 N aqueous sodium hydroxide solution (5.6 mL, 5.7 mmol, 9.0 eq.). Compound 63 was obtained as a yellow powder (0.043 g, 15% yield). ¹H NMR (400 MHz, DMSO-d₆) δ 1.32-1.45 (m, 2H), 1.92-2.06 (m, 2H), 7.44 (t, J=7.71 Hz, 1H), 7.51-7.68 (m, 3H), 7.80 (d, J=7.07 Hz, 1H), 7.85 (d, J=7.83 Hz, 1H), 9.08 (d, J=8.59 Hz, 1H)

### Compound 64: 3-hydroxy-6,8-dimethyl-2-(1-phenylcyclopropyl)quinoline-4-carboxylic acid

Following the procedure described for the preparation of Compound 14, 5,7-dimethylindoline-2,3-dione (0.50 g, 2.86 mmol, 1.0 eq.) was reacted with intermediate 8 (2-oxo-2-(1-phenylcyclopropyl)ethyl acetate, 0.81 g, 3.71 mmol, 1.3 eq.) in the presence of 10.0 N aqueous sodium hydroxide solution (2.5 mL, 25.7 mmol, 9.0 eq.). Compound 64 was obtained as a yellow powder (0.492 g, 52% yield). ¹H NMR (400 MHz, DMSO-d₆) δ 1.28-1.39 (m, 2H), 1.45-1.59 (m, 2H), 2.44 (s, 3H), 2.69 (s, 3H), 7.05-7.17 (m, 3H), 7.17-7.25 (m, 2H), 7.29 (s, 1H), 8.17 (s, 1H).

### Compound 65: 8-ethyl-2-(1-(4-fluorophenyl)cyclopropyl)-3-hydroxyquinoline-4-carboxylic acid

Following the procedure described for the preparation of Compound 14, intermediate 63 (7-ethylindoline-2,3-dione, 0.139 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 65 was obtained as a yellow powder (0.055 g, 20% yield). ¹H NMR (400 MHz, DMSO-d₆) δ 1.24-1.38 (m, 5H), 1.42-1.54 (m, 2H), 3.22 (q, J=7.33 Hz, 2H), 6.92-7.12 (m, 2H), 7.19-7.31 (m, 2H), 7.39-7.42 (m, 1H), 7.44-7.50 (m, 1H), 8.45 (d, J=8.34 Hz, 1H).

### Compound 66: 7-ethyl-2-(1-(4-fluorophenyl)cyclopropyl)-3-hydroxyquinoline-4-carboxylic acid

Following the procedure described for the preparation of Compound 14, 6-ethylindoline-2,3-dione (0.139 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 66 was obtained as a yellow powder (0.050 g, 18% yield). ¹H NMR (400 MHz, DMSO-d₆) δ 1.28 (t, J=7.58 Hz, 3H), 1.36 (t, J=5.05 Hz, 2H), 1.43-1.54 (m, 2H), 2.78 (q, J=7.66 Hz, 2H), 7.05 (t, J=8.84 Hz, 2H), 7.27 (dd, J=8.34, 5.56 Hz, 2H), 7.48 (d, J=8.84 Hz, 1H), 7.79 (s, 1H), 8.80 (s, 1H).

### Compound 67: 6-chloro-2-(1-(4-fluorophenyl)cyclopropyl)-3-hydroxyquinoline-4-carboxylic acid

Following the procedure described for the preparation of Compound 14, 5-chloroindoline-2,3-dione (0.145 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 67 was obtained as a yellow powder (0.130 g, 45% yield). ¹H NMR (400 MHz, DMSO-d₆) δ 1.32 (dd, J=8.00, 4.00 Hz, 4H), 1.45 (dd, J=8.00, 4.00 Hz, 4H), 7.04 (dd, J=8.84, 5.56 Hz, 2H), 7.24 (dd, J=8.84, 5.56 Hz, 2H), 7.49 (dd, J=8.84, 2.53 Hz, 1H), 7.93 (d, J=8.84 Hz, 1H), 9.01 (s, 1H).

### Compound 68: 7-chloro-2-(1-(4-fluorophenyl)cyclopropyl)-3-hydroxyquinoline-4-carboxylic acid

Following the procedure described for the preparation of Compound 14, 6-chloroindoline-2,3-dione (0.145 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 68 was obtained as a yellow powder (0.109 g, 38% yield). ¹H NMR (400 MHz, DMSO-d₆) δ 1.33 (dd, J=8.00, 4.00 Hz, 2H), 1.45 (dd, J=8.00, 4.00 Hz, 2H), 6.97-7.10 (m, 2H), 7.15-7.34 (m, 2H), 7.57 (dd, J=9.09, 2.27 Hz, 1H), 7.97 (d, J=2.53 Hz, 1H), 8.87 (d, J=9.09 Hz, 1H).

### Compound 69: 2-(1-(4-fluorophenyl)cyclopropyl)-3-hydroxy-6,8-dimethylquinoline-4-carboxylic acid

Following the procedure described for the preparation of Compound 14, 5,7-dimethylindoline-2,3-dione (0.140 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 69 was obtained as a yellow powder (0.147 g, 52% yield). ¹H NMR (400 MHz, DMSO-d₆) δ 1.32 (dd, J=8.00, 4.00 Hz, 2H), 1.49 (dd, J=8.00, 4.00 Hz, 2H), 2.43 (s, 3H), 2.69 (s, 3H), 7.04 (t, J=8.59 Hz, 2H), 7.20 (dd, J=8.59, 5.56 Hz, 2H), 7.28 (s, 1H), 8.18 (s, 1H).

### Compound 70: 6-ethyl-2-(1-(4-fluorophenyl)cyclopropyl)-3-hydroxyquinoline-4-carboxylic acid

Following the procedure described for the preparation of Compound 14, 5-ethylindoline-2,3-dione (0.100 g, 0.6 mmol, 1.0 eq.) was reacted with intermediate 51 (1-(1-(4-fluorophenyl)cyclopropyl)-2-hydroxyethanone, 0.200 g, 1.03 mmol, 1.8 eq.) in the presence of 10.0 N aqueous sodium hydroxide solution (0.93 mL, 9.3 mmol, 9.0 eq.). Compound 70 was obtained as a yellow powder (0.099 g, 47% yield). ¹H NMR (400 MHz, DMSO-d₆) δ 1.25 (t, J=7.58 Hz, 3H), 1.33-1.42 (m, 2H), 1.44-1.54 (m, 2H), 2.78 (q, J=7.49 Hz, 2H), 7.05 (t, J=8.84 Hz, 2H), 7.26 (dd, J=8.21, 5.68 Hz, 2H), 7.46 (dd, J=8.72, 1.64 Hz, 1H), 7.93 (d, J=8.59 Hz, 1H), 8.65 (s, 1H).

### Compound 71: 2-(1-(4-fluorophenyl)cyclopropyl)-3-hydroxy-8-(thiophen-3-yl)quinoline-4-carboxylic acid

Following the procedure described for the preparation of Compound 14, intermediate 54 (7-(thiophen-3-yl)indoline-2,3-dione, 0.183 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 71 was obtained as a yellow powder (0.157 g, 48% yield). ¹H NMR (400 MHz, DMSO-d₆) δ 1.32 (dd, J=4.00, 2.00 Hz, 2H), 1.52 (dd, J=4.00, 2.00 Hz, 2H), 7.57-7.66 (m, 2H), 7.72 (d, J=5.05 Hz, 1H), 7.78 (d, J=6.57 Hz, 1H), 8.08 (d, J=2.27 Hz, 1H), 8.49 (d, J=8.59 Hz, 1H).

### Compound 72: 6-bromo-2-(1-(4-fluorophenyl)cyclopropyl)-3-hydroxyquinoline-4-carboxylic acid

Following the procedure described for the preparation of Compound 14, 5-bromoindoline-2,3-dione (0.181 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 72 was obtained as a yellow powder (0.145 g, 45% yield). ¹H NMR (400 MHz, DMSO-d₆) δ 1.33 (dd, J=4.00, 2.00 Hz, 2H), 1.46 (dd, J=4.00, 2.00 Hz, 2H), 7.04 (t, J=8.84 Hz, 2H), 7.24 (dd, J=8.72, 5.43 Hz, 2H), 7.63 (dd, J=8.84, 2.02 Hz, 1H), 7.88 (d, J=8.84 Hz, 1H), 9.10 (d, J=1.26 Hz, 1H).

### Compound 73: 8-chloro-2-(1-(4-fluorophenyl)cyclopropyl)-3-hydroxyquinoline-4-carboxylic acid

Following the procedure described for the preparation of Compound 14, 7-chloroindoline-2,3-dione (0.145 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 73 was obtained as a yellow powder (0.045 g, 16% yield). ¹H NMR (400 MHz, DMSO-d₆) δ 1.34 (dd, J=8.00, 4.00 Hz, 2H), 1.53 (dd, J=8.00, 4.00 Hz, 2H), 7.04 (t, J=8.97 Hz, 2H), 7.21 (dd, J=8.72, 5.43 Hz, 2H), 7.51 (t, J=8.21 Hz, 1H), 7.69 (d, J=7.58 Hz, 1H), 8.69 (d, J=8.59 Hz, 1H).

### Compound 74: 7-bromo-2-(1-(4-fluorophenyl)cyclopropyl)-3-hydroxyquinoline-4-carboxylic acid

Following the procedure described for the preparation of Compound 14, 6-bromoindoline-2,3-dione (0.181 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 74 was obtained as a yellow powder (0.142 g, 44% yield). ¹H NMR (400 MHz, DMSO-d₆) δ 1.33 (dd, J=4.00, 2.00 Hz, 2H), 1.45 (dd, J=8.00, 4.00 Hz, 2H), 7.04 (t, J=8.97 Hz, 2H), 7.24 (dd, J=8.84, 5.56 Hz, 2H), 7.68 (dd, J=9.35, 2.27 Hz, 1H), 8.13 (d, J=2.27 Hz, 1H), 8.78 (d, J=9.35 Hz, 1H).

### Compound 75: 8-bromo-2-(1-(4-fluorophenyl)cyclopropyl)-3-hydroxyquinoline-4-carboxylic acid

Following the procedure described for the preparation of Compound 14, intermediate 71 (7-bromoindoline-2,3-dione, 0.181 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 75 was obtained as a yellow powder (0.160 g, 50% yield). ¹H NMR (400 MHz, DMSO-d₆) δ 1.33 (dd, J=4.00, 2.00 Hz, 4H), 1.53 (dd, J=8.00, 4.00 Hz, 2H), 7.04 (t, J=8.72 Hz, 2H), 7.21 (dd, J=8.59, 5.56 Hz, 2H), 7.40 (t, J=8.00 Hz, 1H), 7.84 (dd, J=7.45, 1.14 Hz, 1H), 8.86 (d, J=8.84 Hz, 1H).

### Compound 76: 2-(1-(4-fluorophenyl)cyclopropyl)-8-(1,1,1,3,3,3-hexafluoro-2-hydroxypropan-2-yl)-3-hydroxyquinoline-4-carboxylic acid

Following the procedure described for the preparation of Compound 14, intermediate 16 (7-(1,1,1,3,3,3-hexafluoro-2-hydroxypropan-2-yl)indoline-2,3-dione, 0.250 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 76 was obtained as a yellow powder (0.152 g, 39% yield). ¹H NMR (400 MHz, DMSO-d₆) δ 1.39 (d, J=4.29 Hz, 4H), 7.09 (t, J==8.72 Hz, 2H), 7.29 (dd, J=8.59, 5.56 Hz, 2H), 7.67 (t, J=7.60 Hz, 1H), 7.70-7.80 (m, 1H), 9.11 (d, J=8.34 Hz, 1H).

### Compound 77: 2-(1-4-fluorophenyl)cyclopropyl)-3-hydroxy-8-phenylquinoline-4-carboxylic acid

Following the procedure described for the preparation of Compound 14, intermediate 11 (7-phenylindoline-2,3-dione, 0.178 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 77 was obtained as a yellow powder (0.132 g, 41% yield). ¹H NMR (400 MHz, DMSO-d₆) δ 1.25 (dd, J=8.00, 4.00 Hz, 2H), 1.45 (dd, J=8.00, 4.00 Hz, 2H), 7.01 (t, J=8.97 Hz, 2H), 7.11 (dd, J=8.72, 5.43 Hz, 2H), 7.39 (t, J=7.33 Hz, 1H), 7.48 (t, J=7.45 Hz, 2H), 7.55-7.61 (m, 1H), 7.65 (t, J=7.71 Hz, 3H), 8.58 (dd, J=8.46, 1.14 Hz, 1H).

### Compound 78: 2-(1-(4-fluorophenyl)cyclopropyl)-3-hydroxy-8-methylquinoline-4-carboxylic acid

Following the procedure described for the preparation of Compound 14, intermediate 73 (7-methylindoline-2,3-dione, 0.129 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 78 was obtained as a yellow powder (0.122 g, 45% yield). ¹H NMR (400 MHz, DMSO-d₆) δ 1.34 (dd, J=4.00, 2.00 Hz, 32H), 1.51 (dd, J=4.00, 2.00 Hz, 2H), 2.73 (s, 3H), 7.04 (t, J=8.97 Hz, 2H), 7.22 (dd, J=8.72, 5.43 Hz, 2H), 7.35-7.66 (m, 2H), 8.41 (s, 1H).

### Compound 79: 2-(1-(4-fluorophenyl)cyclopropyl)-3-hydroxy-6-methoxyquinoline-4-carboxylic acid

Following the procedure described for the preparation of Compound 14, 5-methoxyindoline-2,3-dione (0.142 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 79 was obtained as a yellow powder (0.053 g, 19% yield). ¹H NMR (400 MHz, DMSO-d₆) δ 1.36 (s, 2H), 1.47 (s, 2H), 7.05 (t, J=8.84 Hz, 2H), 7.22 (dd, J=9.22, 2.65 Hz, 2H), 7.28 (dd, J=7.33, 5.05 Hz, 1H), 7.92 (d, J=9.09 Hz, 1H), 8.42 (s, 1H).

### Compound 80: 2-(1-(4-fluorophenyl)cyclopropyl)-3-hydroxy-7,8,9,10-tetrahydrobenzo[h]quinoline-4-carboxylic acid

Following the procedure described for the preparation of Compound 14, intermediate 3 (6,7,8,9-tetrahydro-1H-benzo[g]indole-2,3-dione, 0.202 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 80 was obtained as a yellow powder (0.034 g, 11% yield). ¹H NMR (400 MHz, DMSO-d₆) δ 1.32 (dd, J=4.00, 2.00 Hz, 2H), 1.48 (dd, J=4.00, 2.00 Hz, 2H), 1.73-1.92 (m, 4H), 2.84 (t, J=5.68 Hz, 2H), 3.26 (t, J=5.68 Hz, 2H), 7.04 (t, J=8.84 Hz, 2H), 7.22 (dd, J=8.72, 5.43 Hz, 2H), 7.28 (d, J=8.84 Hz, 1H), 8.34 (d, J=8.84 Hz, 1H).

### Compound 81: 2-(1-(4-fluorophenyl)cyclopropyl)-3-hydroxy-7,8-dimethylquinoline-4-carboxylic acid

Following the procedure described for the preparation of Compound 14, intermediate 4 (6,7-dimethylindoline-2,3-dione, 0.140 g, 0.8 mmol, 1.0 eq.) was reacted with intermediate 51 (1-(1-(4-fluorophenyl)cyclopropyl)-2-hydroxyethanone, 0.200 g, 1.03 mmol, 1.3 eq.) in the presence of 10.0 N aqueous sodium hydroxide solution (0.93 mL, 9.3 mmol, 9.0 eq.). Compound 81 was obtained as a yellow powder (0.105 g, 37% yield). ¹H NMR (400 MHz, DMSO-d₆) δ 1.34 (dd, J=4.00, 2.00 Hz, 2H), 1.51 (dd, J=4.00, 2.00 Hz, 2H), 2.42 (s, 3H), 2.70 (s, 3H), 7.04 (t, J=8.84 Hz, 2H), 7.21 (dd, J=8.59, 5.56 Hz, 2H), 7.40 (d, J=8.84 Hz, 1H), 8.31 (d, J=8.59 Hz, 1H).

### Compound 82: 8-ethyl-2-(1-p-tolylcyclopropyl)-3-hydroxyquinoline-4-carboxylic acid

Following the procedure described for the preparation of Compound 14, intermediate 63 (7-ethylindoline-2,3-dione, 0.140 g, 0.8 mmol, 1.0 eq.) was reacted with intermediate 77 (1-(1-(4-methylphenyl)cyclopropyl)-2-hydroxyethanone, 0.297 g, 1.03 mmol, 1.3 eq. at 66% purity) in the presence of 10.0 N aqueous sodium hydroxide solution (0.93 mL, 9.3 mmol, 9.0 eq.). Compound 82 was obtained as a yellow powder (0.129 g, 46% yield). ¹H NMR (400 MHz, DMSO-d₆) δ 1.23-1.36 (m, 5H), 1.40-1.60 (m, 2H), 2.22 (s, 3H), 3.23 (q, J=7.49 Hz, 2H), 6.89-7.14 (m, 4H), 7.40-7.44 (m, 1H), 7.48 (t, J=7.60 Hz, 1H), 8.40 (d, J=7.33 Hz, 1H).

### Compound 83: 8-methyl-2-(1-p-tolylcyclopropyl)-3-hydroxyquinoline-4-carboxylic acid

Following the procedure described for the preparation of Compound 14, intermediate 73 (7-methylindoline-2,3-dione, 0.129 g, 0.8 mmol, 1.0 eq.) was reacted with intermediate 77 (1-(1-(4-methylphenyl)cyclopropyl)-2-hydroxyethanone, 0.297 g, 1.03 mmol, 1.3 eq. at 66% purity) in the presence of 10.0 N aqueous sodium hydroxide solution (0.93 mL, 9.3 mmol, 9.0 eq.). Compound 83 was obtained as a yellow powder (0.138 g, 52% yield). ¹H NMR (400 MHz, DMSO-d₆) δ 1.31 (dd, J=8.00, 4.00 Hz, 2H), 1.49 (dd, J=8.00, 4.00 Hz, 2H), 2.22 (s, 3H), 2.73 (s, 3H), 6.85-7.13 (m, 4H), 7.35-7.53 (m, 2H), 8.39 (d, J=8.34 Hz, 1H).

### Compound 84: 3-hydroxy-6,8-dimethyl-2-(1-p-tolylcyclopropyl)quinoline-4-carboxylic acid

Following the procedure described for the preparation of Compound 14, 5,7-dimethylindoline-2,3-dione (0.140 g, 0.8 mmol, 1.0 eq.) was reacted with intermediate 77 (1-(1-(4-methylphenyl)cyclopropyl)-2-hydroxyethanone, 0.297 g, 1.03 mmol, 1.3 eq. at 66% purity) in the presence of 10.0 N aqueous sodium hydroxide solution (0.93 mL, 9.3 mmol, 9.0 eq.). Compound 84 was obtained as a yellow powder (0.154 g, 55% yield). ¹H NMR (400 MHz, DMSO-d₆) δ 1.29 (dd, J=4.00, 2.00 Hz, 2H), 1.46 (dd, J=4.00, 2.00 Hz, 2H), 2.22 (s, 3H), 2.43 (s, 3H), 2.69 (s, 3H), 6.83-7.09 (m, 4H), 7.27 (s, 1H), 8.20 (s, 1H)

### Compound 85: 8-(1,1,1,3,3,3-hexafluoro-2-hydroxypropan-2-yl)-3-hydroxy-2-(1-p-tolylcyclopropyl)quinoline-4-carboxylic acid

Following the procedure described for the preparation of Compound 14, intermediate 16 (7-(1,1,1,3,3,3-hexafluoro-2-hydroxypropan-2-yl)indoline-2,3-dione, 0.250 g, 0.8 mmol, 1.0 eq.) was reacted with intermediate 77 (1-(1-(4-methylphenyl)cyclopropyl)-2-hydroxyethanone, 0.297 g, 1.03 mmol, 1.3 eq. at 66% purity) in the presence of 10.0 N aqueous sodium hydroxide solution (0.93 mL, 9.3 mmol, 9.0 eq.). Compound 85 was obtained as a yellow powder (0.118 g, 30% yield). ¹H NMR (400 MHz, DMSO-d₆) δ 1.32 (d, J=8.00 Hz, 2H), 1.37 (d, J=8.00 Hz, 2H), 2.22 (s, 3H), 6.96-7.08 (m, 2H), 7.09-7.15 (m, 2H), 7.66 (t, J=7.60 Hz, 1H), 7.72 (d, J=7.60 Hz, 1H), 9.15 (d, J=7.83 Hz, 1H).

### Compound 86: 3-hydroxy-8-isopropyl-2-(1-p-tolylcyclopropyl)quinoline-4-carboxylic acid

Following the procedure described for the preparation of compound 14, intermediate 5 (7-isopropylindoline-2,3-dione, 0.1787 g, 0.946 mmol, 1.0 equiv) was reacted with intermediate 77 (1-(1-(4-methylphenyl)cyclopropyl)-2-hydroxyethanone, 0.297 g, 1.03 mmol, 1.3 eq at 66% purity) in the presence of 10.0 N aqueous sodium hydroxide solution (0.93 mL, 9.3 mmol, 9.0 eq). Compound 86 was obtained as a yellow powder. ¹H NMR (400 MHz, DMSO-d₆) δ 1.31 (dd, J=8.00, 4.00 Hz, 2H), 1.34 (d, J=7.07 Hz, 6H), 1.45 (dd, J=8.00, 4.00 Hz, 2H), 2.22 (s, 3H), 4.12-4.35 (m, 1H), 7.02 (d, J=7.90 Hz, 2H), 7.08 (d, J=7.90 Hz, 2H), 7.42 (d, J=6.32 Hz, 1H), 7.50 (t, J=7.63 Hz, 1H), 8.44 (d, J=8.59 Hz, 1H).

### Compound 87: 8-ethyl-3-hydroxy-2-(1-(4-(trifluoromethyl)phenyl)cyclopropyl)quinoline-4-carboxylic acid

Following the procedure described for the preparation of Compound 14, intermediate 63 (7-ethylindoline-2,3-dione, 0.200 g, 1.14 mmol, 1.0 eq.) was reacted with intermediate 21 (2-hydroxy-1-(1-(4-(trifluoromethyl)phenyl)cyclopropyl)ethanone, 0.3624 g, 1.484 mmol, 1.3 eq.) in the presence of 10.0 M aqueous sodium hydroxide solution (1.026 mL, 10.26 mmol, 9.0 eq.). Compound 87 was obtained as a yellow powder (0.0613 g, 13.4% yield). ¹H NMR (400 MHz, CDCl₃) δ 1.18-1.29 (m, 2H), 1.36 (t, J=7.20 Hz, 3H), 1.40-1.47 (m, 2H), 3.23-3.33 (m, 2H), 7.40-7.46 (m, 3H), 7.46-7.54 (m, 4H), 8.58 (d, J=8.45 Hz 1H).

### Compound 88: 3-hydroxy-8-isopropyl-2-(1-(4-(trifluoromethyl)phenyl)cyclopropyl)quinoline-4-carboxylic acid

Following the procedure described for the preparation of Compound 14, intermediate 5, (7-isopropylindoline-2,3-dione, 0.1787 g, 0.946 mmol, 1.0 eq.) was reacted with intermediate 21 (2-hydroxy-1-(1-(4-(trifluoromethyl)phenyl)cyclopropyl)ethanone, 0.300 g, 1.23 mmol, 1.3 eq.) in the presence of 10.0 M aqueous sodium hydroxide solution (0.85 mL, 8.5 mmol, 9.0 eq.). Compound 88 was obtained as a yellow powder (0.0320 g, 8.14% yield). ¹H NMR (400 MHz, DMSO-d₆) δ 1.34 (d, J=7.07 Hz, 6H), 1.44-1.51 (m, 2H), 1.57-1.64 (m, 2H), 4.20-4.31 (m, 1H), 7.33 (d, J=8.08 Hz, 2H), 7.47 (d, 2H), 7.52-7.62 (m, 3H), 8.36 (d, J=8.59 Hz, 1H).

### Compound 89: 7-ethyl-3-hydroxy-2-(1-(4-(trifluoromethyl)phenyl)cyclopropyl)quinoline-4-carboxylic acid

Following the procedure described for the preparation of Compound 14, 6-ethylindoline-2,3-dione (0.1752 g, 1.0 mmol, 1.0 eq.) was reacted with intermediate 21 (2-hydroxy-1-(1-(4-(trifluoromethyl)phenyl)cyclopropyl)ethanone, 0.3175 g, 1.30 mmol, 1.3 eq.) in the presence of 10.0 M aqueous sodium hydroxide solution (0.90 mL, 9.00 mmol, 9.0 eq.). Compound 89 was obtained as a yellow powder (0.0314 g, 7.82% yield). ¹H NMR (400 MHz, DMSO-d₆) δ 1.28 (t, J=7.58 Hz, 3H), 1.44-1.51 (m, 2H), 1.55-1.62 (m, 2H), 2.79 (q, J=7.49 Hz, 2H), 7.33 (d, J=8.08 Hz, 2H), 7.50 (dd, J=8.84, 1.52 Hz, 1H), 7.58 (d, J=8.34 Hz, 2H), 7.81 (s, 1H), 8.69 (d, 1H).

### Compound 90: 3-hydroxy-6-(trifluoromethoxy)-2-(1-(4-(trifluoromethyl)phenyl)cyclopropyl)quinoline-4-carboxylic acid

Following the procedure described for the preparation of Compound 14, 5-(trifluoromethoxy)indoline-2,3-dione (0.1893 g, 0.819 mmol, 1.0 eq.) was reacted with intermediate 21 (2-hydroxy-1-(1-(4-(trifluoromethyl)phenyl)cyclopropyl)ethanone, 0.260 g, 1.06 mmol, 1.3 eq.) in the presence of 10.0 M aqueous sodium hydroxide solution (0.90 mL, 9.0 mmol, 9.0 eq.). Compound 90 was obtained as a yellow powder (0.0637 g, 17.0% yield). ¹H NMR (400 MHz, DMSO-d₆) δ 1.43-1.52 (m, 2H), 1.56-1.64 (m, 2H), 7.33 (d, J=8.08 Hz, 2H), 7.50-7.62 (m, 3H), 8.11 (d, J=9.09 Hz, 1H), 8.78 (d, J=1.52 Hz, 1H).

### Compound 91: 3-hydroxy-8-(thiophen-3-yl)-2-(1-(4-(trifluoromethyl)phenyl)cyclopropyl)quinoline-4-carboxylic acid

Following the procedure described for the preparation of Compound 14, intermediate 54 (7-(thiophen-3-yl)indoline-2,3-dione, 0.18775 g, 0.819 mmol, 1.0 eq.) was reacted with intermediate 21 (2-hydroxy-1-(1-(4-(trifluoromethyl)phenyl)cyclopropyl)ethanone, 0.260 g, 1.06 mmol, 1.3 eq.) in the presence of 10.0 M aqueous sodium hydroxide solution (0.90 mL, 9.0 mmol, 9.0 eq.). Compound 91 was obtained as a yellow powder (0.0835 g, 22.38% yield). ¹H NMR (400 MHz, DMSO-d₆) δ 1.40-1.47 (m, 2H), 1.59-1.67 (m, 2H), 7.56 (d, J=8.34 Hz, 2H), 7.60-7.65 (m, 2H), 7.69 (dd, J=5.05, 1.26 Hz, 1H), 7.77 (dd, J=7.33, 1.26 Hz, 1H), 8.04 (dd, J=3.03, 1.26 Hz, 1H), 8.54 (d, 2H).

### Compound 92: 3-hydroxy-8-phenyl-2-(1-(4-(trifluoromethyl)phenyl)cyclopropyl)quinoline-4-carboxylic acid

Following the procedure described for the preparation of Compound 14, intermediate 11 (7-phenylindoline-2,3-dione, 0.033 g, 0.142 mmol, 1.0 eq.) was reacted with intermediate 21 (2-hydroxy-1-(1-(4-(trifluoromethyl)phenyl)cyclopropyl)ethanone, 0.045 g, 0.184 mmol, 1.3 eq.) in the presence of 10.0 M aqueous sodium hydroxide solution (0.13 mL, 1.3 mmol, 9.0 eq.). Compound 92 was obtained as a yellow powder (0.016 g, 25.07% yield). ¹H NMR (400 MHz, DMSO-d₆) δ 1.32-1.38 (m, 2H), 1.48-1.59 (m, 2H), 7.21 (d, J=8.08 Hz, 2H), 7.38 (t, J=7.45 Hz, 2H), 7.47 (t, J=7.58 Hz, 2H), 7.47 (t, 1H), 7.51-7.56 (m, 2H), 7.60-7.67 (m, 3H), 7.71 (d, 1H), 8.78 (d, J=8.08 Hz, 1H).

### Compound 93: 3-hydroxy-2-(1-(4-(trifluoromethyl)phenyl)cyclopropyl)-7,8,9,10-tetrahydrobenzo[h]quinoline-4-carboxylic acid

Following the procedure described for the preparation of Compound 14, intermediate 3 (6,7,8,9-tetrahydro-1H-benzo[g]indole-2,3-dione, 0.2269 g, 1.127 mmol, 1.0 eq.) was reacted with intermediate 21 (2-hydroxy-1-(1-(4-(trifluoromethyl)phenyl)cyclopropyl)ethanone, 0.358 g, 1.466 mmol, 1.3 eq.) in the presence of 10.0 M aqueous sodium hydroxide solution (1.01 mL, 10.1 mmol, 9.0 eq.). Compound 93 was obtained as a yellow powder (0.014 g, 5% yield). ¹H NMR (400 MHz, DMSO-d₆) δ 1.18-1.27 (m, 2H), 1.39-1.45 (m, 2H), 1.49-1.55 (m, 2H), 1.55-1.62 (m, 2H), 1.76-1.90 (m, 4H), 7.25 (d, J=8.84 Hz, 1H), 7.30 (d, J=7.83 Hz, 1H), 7.56 (d, J=8.34 Hz, 1H), 7.60-7.68 (m, 2H), 7.68-7.76 (m, 2H), 8.54 (d, J=9.35 Hz, 1H).

### Compound 94: 2-(1-(4-chlorophenyl)cyclopropyl)-3-hydroxy-6-methyl-8-(trifluoromethyl)quinoline-4-carboxylic acid

Following the procedure described for the preparation of Compound 14, intermediate 80 was reacted intermediate 78 in the presence of sodium hydroxide solution. Compound 94 was obtained as a fluffy, pale yellow solid. ¹H NMR (400 MHz, DMSO-d₆) δ 1.35-1.39 (m, 2H), 1.50-1.54 (m, 2H), 2.55 (s, 3H), 7.18 (dt, J=8.8, 2.5 Hz, 2H), 7.29 (dt, J=8.8, 2.5 Hz, 2H), 7.83 (s, 1H), 8.60 (s, 1H).

### Compound 95: 6-chloro-2-(1-(4-chlorophenyl)cyclopropyl)-3-hydroxy-8-(trifluoromethyl)quinoline-4-carboxylic acid

Following the procedure described for the preparation of Compound 14, intermediate 82 (0.63 g, 2.5 mmol) was reacted with intermediate 78 (0.70 g, 3.3 mmol). Compound 95 was obtained as a fluffy, pale yellow solid (28 mg, 2.5% yield). ¹H NMR (400 MHz, DMSO-d₆) δ 1.33-1.37 (m, 2H), 1.48-1.52 (m, 2H), 7.18 (ddd, 2H), 7.28 (ddd, J=8.8, 2.5, 2.3 Hz, 2H), 7.90 (d, J=2.3 Hz, 1H), 9.20 (d, J=2.0 Hz, 1H).

### Compound 96: 2-(1-(4-chlorophenyl)cyclopropyl)-3-hydroxy-6-phenyl-8-(trifluoromethyl)quinoline-4-carboxylic acid

Following the procedure described for the preparation of Compound 14, intermediate 83 (0.89 g, 3.1 mmol) was reacted with intermediate 78 (0.84 g, 4.0 mmol). Compound 96 was obtained as a fluffy, bright yellow solid (95 mg, 6.4% yield). ¹H NMR (400 MHz, DMSO-d₆) δ 1.37-1.41 (m, 2H), 1.53-1.57 (m, 2H), 7.20 (ddd, J=8.9, 2.7, 2.3 Hz, 2H), 7.28-7.32 (m, 2H), 7.45-7.50 (m, 1H), 7.56 (t, J=7.6 Hz, 2H), 7.77-7.81 (m, 2H), 8.17 (d, J=2.0 Hz, 1H), 9.20 (d, J=1.8 Hz, 1H).

### Compound 97: 2-(1-(4-chlorophenyl)cyclopropyl)-3-hydroxy-8-methyl-6-(trifluoromethyl)quinoline-4-carboxylic acid

Following the procedure described for the preparation of Compound 14, intermediate 88 (0.259 g, 1.13 mmol) was reacted with intermediate 78 (0.31 g, 1.5 mmol). Compound 97 was obtained as a fluffy, pale yellow solid (31.6 mg, 6.6% yield). ¹H NMR (400 MHz, DMSO-d₆) δ 1.36-1.41 (m, 2H), 1.55-1.59 (m, 2H), 2.80 (s, 3H), 7.15-7.20 (m, 2H), 7.28 (ddd, J=8.9, 2.5, 2.2 Hz, 2H), 7.69 (s, 1H), 8.99 (s, 1H).

### Compound 98: 2-(1-(4-chlorophenyl)cyclopropyl-6-ethyl-3-hydroxy-8-(trifluoromethyl)quinoline-4-carboxylic acid

Following the procedure described for the preparation of Compound 14, intermediate 89 (0.271 g, 1.11 mmol) was reacted with intermediate 78 (0.31 g, 1.5 mmol). Compound 98 was obtained as a fluffy, pale yellow solid (66 mg, 14% yield). ¹H NMR (400 MHz, DMSO-d₆) δ 1.27 (t, J=7.6 Hz, 3H), 1.33-1.39 (m, 2H), 1.48-1.54 (m, 2H), 2.85 (q, J=7.4 Hz, 2H), 7.14-7.20 (m, 2H), 7.25-7.32 (m, 2H), 7.85 (d, J=1.5 Hz, 1H), 8.67 (s, 1H).

### Compound 99: 2-(1-(4-chlorophenyl)cyclopropyl)-8-ethyl-3-hydroxy-6-(trifluoromethyl)quinoline-4-carboxylic acid

Following the procedure described for the preparation of Compound 14, intermediate 90 (0.377 g, 1.55 mmol) was reacted with intermediate 78 (0.39 g, 1.9 mmol). Compound 99 was obtained as a fluffy, pale yellow solid (106 mg, 16% yield). ¹H NMR (400 MHz, DMSO-d₆) δ 1.33 (t, J=7.5 Hz, 3H), 1.36-1.41 (m, 2H), 1.52-1.57 (m, 2H), 3.28 (q, J=7.4 Hz, 2H), 7.17-7.22 (m, 2H), 7.26-7.31 (m, 2H), 7.64 (d, J=2.0 Hz, 1H), 9.03 (s, 1H).

### Compound 100: 2-(1-(4-chlorophenyl)cyclopropyl)-3-hydroxy-8-phenyl-6-(trifluoromethyl)quinoline-4-carboxylic acid

Following the procedure described for the preparation of Compound 14, intermediate 91 (0.521 g, 1.79 mmol) was reacted with intermediate 78 (0.45 g, 2.2 mmol). Compound 100 was obtained as a fluffy yellow solid (196 mg, 23% yield). ¹H NMR (400 MHz, DMSO-d₆) δ 1.28-1.33 (m, 2H), 1.49-1.55 (m, 2H), 7.08 (d, J=8.6 Hz, 2H), 7.25 (d, J=8.6 Hz, 2H), 7.45 (t, J=7.3 Hz, 1H), 7.52 (t, J=7.3 Hz, 2H), 7.69 (d, J=7.1 Hz, 2H), 7.77 (d, J=1.8 Hz, 1H), 9.14 (s, 1H).

### Compound 101: 3-hydroxy-6-methyl-2-(1-phenylcyclopropyl)-8-(trifluoromethyl)quinoline-4-carboxylic acid

Following the procedure described for the preparation of Compound 14, intermediate 80 (0.415 g, 1.81 mmol) was reacted with intermediate 92 (0.42 g, 2.4 mmol). Compound 101 was obtained as a fluffy yellow solid (70 mg, 10% yield). ¹H NMR (400 MHz, DMSO-d₆) δ 1.33-1.39 (m, 2H), 1.45-1.52 (m, 2 μl), 2.55 (s, 3H), 7.11-7.20 (m, 3H), 7.21-7.27 (m, 2H), 7.83 (d, J=1.3 Hz, 1H), 8.61 (s, 1H).

### Compound 102: 3-hydroxy-6-phenyl-2-(1-Phenylcyclopropyl)-8-(trifluoromethyl)quinoline-4-carboxylic acid

Following the procedure described for the preparation of Compound 14, intermediate 83 (0.504 g, 1.73 mmol) was reacted with intermediate 92 (0.40 g, 2.3 mmol). Compound 102 was obtained as a fluffy bright yellow solid (75 mg, 9.7% yield). ¹H NMR (400 MHz, DMSO-d₆) δ 1.36-1.42 (m, 2H), 1.50-1.55 (m, 2H), 7.13-7.21 (m, 3H), 7.22-7.28 (m, 2H), 7.48 (t, J=7.2 Hz, 1H), 7.57 (t, J=7.7 Hz, 2H), 7.79 (d, J=7.1 Hz, 2H), 8.18 (d, J=1.8 Hz, 1H), 9.20 (d, J=1.8 Hz, 1H).

### Compound 103: 6-bromo-2-(1-(4-chlorophenyl)cyclopropyl)-3-hydroxy-8-(trifluoromethyl)quinoline-4-carboxylic acid

Following the procedure described for the preparation of Compound 14, intermediate 93 (0.438 g, 1.49 mmol) was reacted with intermediate 78 (0.41 g, 1.9 mmol). Compound 103 was obtained as a fluffy yellow solid (26 mg, 3.5% yield). ¹H NMR (400 MHz, DMSO-d₆) δ 1.32-1.37 (m, 2H), 1.48-1.52 (m, 2H), 7.18 (ddd, J=8.9, 2.5, 2.2 Hz, 2H), 7.28 (ddd, J=8.8, 2.4, 2.2 Hz, 2H), 7.96 (d, J=2.0 Hz, 1H), 9.41 (d, J=2.0 Hz, 1H).

### Compound 104: 6-ethyl-3-hydroxy-2-(1-phenylcyclopropyl)-8-(trifluoromethyl)quinoline-4-carboxylic acid

Following the procedure described for the preparation of Compound 14, intermediate 89 (0.417 g, 1.71 mmol) was reacted with intermediate 92 (0.39 g, 2.2 mmol). Compound 104 was obtained as a fluffy yellow solid (26 mg, 3.8% yield). ¹H NMR (400 MHz, DMSO-d₆) δ 1.27 (t, J=7.5 Hz, 3H), 1.32-1.38 (m, 2H), 1.46-1.51 (m, 2H), 2.85 (q, J=7.6 Hz, 2H), 7.11-7.18 (m, 3H) 7.20-7.26 (m, 2H), 7.84 (d, J=1.8 Hz, 1H), 8.67 (s, 1H).

### Compound 105: 3-hydroxy-2-(1-(4-chlorophenyl)cyclopropyl)-6,8-bis(trifluoromethyl)quinoline-4-carboxylic acid

Following the procedure described for the preparation of Compound 14, intermediate 96 (0.431 g, 1.52 mmol) was reacted with intermediate 78 (0.349 g, 1.98 mmol). Compound 105 was obtained as a fluffy yellow solid (13 mg, 1.9% yield). ¹H NMR (400 MHz, DMSO-d₆) δ 1.33-1.38 (m, 2H), 1.47-1.52 (m, 2H), 7.11-7.17 (m, 1H), 7.17-7.26 (m, 4H), 8.00 (d, J=1.8 Hz, 1H), 9.77 (s, 1H).

### Compound 106: 2-(1-(4-chlorophenyl)cyclopropyl-3-hydroxy-6,8-bis-(trifluoromethyl)quinoline-4-carboxylic acid

Following the procedure described for the preparation of Compound 14, intermediate 96 (0.431 g, 1.52 mmol) was reacted with intermediate 92 (0.417 g, 1.98 mmol). Compound 106 was obtained as a fluffy, pale yellow solid (6.5 mg, 0.9% yield). ¹H NMR (400 MHz, DMSO-d₆) δ 1.32-1.36 (m, 2H), 1.49-1.53 (m, 2H), 7.19 (ddd, J=8.9, 2.7, 2.3 Hz, 2H), 7.28 (ddd, J=9.0, 2.5, 2.4 Hz, 2H), 7.94 (d, J=1.8 Hz, 1H), 9.91 (s, 1H).

### Compound 107: 6-bromo-3-hydroxy-2-(1-phenylcyclopropyl)-8-(trifluoromethyl)quinoline-4-carboxylic acid

Following the procedure described for the preparation of Compound 14, intermediate 93 (0.400 g, 1.36 mmol) was reacted with intermediate 92 (0.31 g, 1.8 mmol). Compound 107 was obtained as a fluffy, pale yellow solid (5.5 mg, 0.9% yield). ¹H NMR (400 MHz, DMSO-d₆) δ 1.28-1.33 (m, 2H), 1.42-1.46 (m, 2H), 7.09-7.15 (m, 1H), 7.15-7.24 (m, 4H), 7.87 (d, J=2.3 Hz, 1H), 9.63 (d, J=2.3 Hz, 1H).

### Compound 108: 2-(1-(4-chlorophenyl)cyclopropyl)-3-hydroxyquinoline-4,8-dicarboxylic acid

Following the procedure described for the preparation of Compound 14, 2,3-dioxoindoline-7-carboxylic acid (0.502 g, 2.63 mmol) was reacted with intermediate 78 (0.72 g, 3.4 mmol). Compound 108 was obtained a fluffy pale yellow solid (8.4 mg, 0.8% yield). ¹H NMR (400 MHz, DMSO-d₆) δ1.41-1.47 (m, 2H), 1.51-1.56 (m, 2H), 7.23-7.28 (m, 2H), 7.28-7.34 (m, 2H), 7.71 (dd, J=8.6, 7.3 Hz, 1H), 8.26 (dd, J=7.2, 1.4 Hz, 1H), 9.23 (d, J=8.1 Hz, 1H).

### Compound 109: 2-[1-(4-chloro-phenyl)-cyclopropyl]-8-cyclopropyl-3-hydroxy-quinoline-4-carboxylic acid

Following the procedure described for the preparation of Compound 14, intermediate 94 (7-cyclopropyl-1H-indole-2,3-dione, 100 mg, 0.53 mmol) was reacted with intermediate 55 (acetic acid 2-[1-(4-chloro-phenyl)-cyclopropyl]-2-oxo-ethyl ester, 130 mg, 0.52 mmol). Compound 109 was obtained as a yellow solid (30 mg, 15.2% yield). ¹H NMR (400 MHz, DMSO-d₆) δ 0.81-0.86 (m, 2H), 1.07-1.14 (m, 2H), 1.34-1.39 (dd, J=6.57, 4.55 Hz, 2H), 1.53-1.57 (dd, J=6.57, 4.04 Hz, 2H), 3.22-3.30 (m, 1H), 7.00 (d, J=7.33 Hz, 1H), 7.16 (d, J=8.84 Hz, 2H), 7.27 (d, J=8.84 Hz, 2H), 7.44 (dd, J=7.33, 7.07 Hz, 1H), 8.37 (d, J=7.07 Hz, 1H).

### Compound 110: 8-cyclopropyl-3-hydroxy-2-(1-phenyl-cyclopropyl)-quinoline-4-carboxylic acid

Following the procedure described for the preparation of Compound 14, intermediate 94 (7-cyclopropyl-1H-indole-2,3-dione, 100 mg, 0.53 mmol) was reacted with intermediate 8 (acetic acid 2-oxo-2-(1-phenyl-cyclopropyl)-ethyl ester, 116 mg, 0.53 mmol). Compound 110 was obtained as a yellow solid (13.0 mg, 7.1% yield). ¹H NMR (400 MHz, DMSO-d₆) δ 0.82-0.87 (m, 2H), 1.08-1.14 (m, 2H), 1.36 (dd, J=6.82, 4.55 Hz, 2H), 1.53 (dd, J=6.82, 5.05 Hz, 2H), 3.22-3.30 (m, 1H), 7.00 (d, J=8.34 Hz, 1H), 7.12-7.17 (m, 2H), 7.19-7.26 (m, 3H), 7.45 (dd, J=8.34, 7.07 Hz, 1H), 8.32-8.39 (d, J=7.07 Hz, 1H).

### Compound 111: 3-hydroxy-2-(1-phenyl-cyclopropylmethyl)-8-trifluoromethyl-quinoline-4-carboxylic acid

Following the procedure described for the preparation of Compound 14, intermediate 6 (7-(trifluoromethyl)indoline-2,3-dione, 35 mg, 0.16 mmol) was reacted with intermediate 98 (1-hydroxy-3-(1-phenyl-cyclopropyl)-propan-2-one, 30 mg, 0.16 mmol). Compound III was obtained as a beige solid (5.0 mg, 8.0% yield). ¹H NMR (400 MHz, MeOD) δ 0.77 (dd, J=6.06, 4.29 Hz, 2H), 0.97 (dd, J=5.81, 4.29 Hz, 2H), 3.32 (s, 2H), 6.91-7.04 (m, 3H), 7.10-7.14 (m, 2H), 7.51 (dd, J=8.84, 7.33 Hz, 1H), 7.78 (d, J=7.33 Hz, 1H), 8.94 (d, J=8.84 Hz, 1H).

### Compound 112: 2-(1-benzyl-cyclopropyl)-3-hydroxy-8-trifluoromethyl-quinoline-4-carboxylic acid

Following the procedure described for the preparation of Compound 14, intermediate 6 (7-(trifluoromethyl)indoline-2,3-dione, 310 mg, 1.44 mmol) was reacted with intermediate 100 (1-(1-Benzyl-cyclopropyl)-2-hydroxy-ethanone, 273 mg, 1.44 mmol). Compound 112 was obtained as a yellow solid (19.0 mg, 3.4% yield). ¹H NMR (400 MHz, MeOD) δ 1.14-1.18 (m, 2H), 1.48-1.53 (m, 2H), 2.24 (s, 2H), 7.21-7.25 (m, 1H), 7.26-7.33 (m, 2H), 7.42-7.47 (m, 2H), 7.76-7.82 (m, 1H), 8.05 (d, J=7.33 Hz, 1H), 9.24 (d, J=8.84 Hz, 1H).

### Compound 113: 3-hydroxy-7,8-dimethyl-2-(1-p-tolyl-cyclopropyl)-quinoline-4-carboxylic acid

Following the procedure described for the preparation of Compound 14, intermediate 4 (6,7-dimethyl-1H-indole-2,3-dione, 263 mg, 1.5 mmol) was reacted with intermediate 77 (2-hydroxy-1-(1-p-tolyl-cyclopropyl)-ethanone, 357 mg, 1.88 mmol). Compound 113 was obtained as a yellow solid (165 mg, 31.7% yield). ¹H NMR (400 MHz, MeOD) δ 1.14-1.18 (m, 2H), 1.48-1.53 (m, 2H), 2.24 (s, 2H), 7.21-7.25 (m, 1H), 7.26-7.33 (m, 2H), 7.42-7.47 (m, 2H), 7.76-7.82 (m, 1H), 8.05 (d, J=7.33 Hz, 1H), 9.24 (d, J=8.84 Hz, 1H).

Other compounds that can act as inhibitors of selectins, such as p-selectin, can be synthesized according to the following procedures.

Compound 114: 3-hydroxy-2-(2-phenylpropan-2-yl)-7,8,9,10-tetrahydrobenzo[h]quinoline-4-carboxylic acid

To a 100 mL round bottom flask equipped with a condenser was added intermediate 3, 6,7,8,9-tetrahydro-1H-benzo[g]indole-2,3-dione (1.76 g, 7.0 mmol, 1.0 equiv) and 40 mL ethanol. To this solution was added 10.0 N aqueous sodium hydroxide solution (6.3 mL, 63.0 mmol, 9.0 equiv) and the mixture was heated to reflux in an oil bath. Stirring was continued at reflux for 30 minutes, at which point a solution of intermediate 2, 3-methyl-2-oxo-3-phenylbutyl acetate (2.0 g, 9.09 mmol, 1.3 equiv) in 10 mL ethanol was added dropwise over 20 minutes. The resulting mixture was allowed to stir at reflux for an additional 12 hours. Upon cooling to room temperature, the mixture was acidified with excess glacial acetic acid and poured into 200 mL water. The suspension was extracted with three 100 mL portions of ethyl acetate, and the combined organic layers were washed with three 200 mL portion of water and 250 mL saturated sodium bicarbonate solution. The organic layer was dried over magnesium sulfate, filtered and the solvent removed in vacuo to give a dark yellow oil. This was purified by reverse-phase HPLC (Base Method 3) and lyophilized to give the desired product as a yellow lyophilized powder (0.0315 g, 1.3%). ¹H NMR (400 MHz, DMSO-d₆) δ ppm 1.79 (s, 6H) 1.81-1.98 (m, 4H) 2.74-2.94 (m, 2H) 3.22-3.46 (m, 2H) 7.08-7.16 (m, 3H) 7.18-7.26 (m, 2H) 7.29 (d, J=8.84 Hz, 1H) 8.36 (d, J=9.09 Hz, 1H).

### Compound 115: 3-hydroxy-7,8-dimethyl-2-(2-phenylpropan-2-yl)quinoline-4-carboxylic acid

Following the procedure described for the preparation of Compound 114, intermediate 2, 3-methyl-2-oxo-3-phenylbutyl acetate (1.65 g, 7.4 mmol, 1.3 equiv), was treated with intermediate 4, 6,7-dimethylindoline-2,3-dione (1.0 g, 5.71 mmol, 1.0 equiv) and 10.0 N aqueous sodium hydroxide solution (5.1 mL, 51.4 mmol, 9.0 equiv). The desired product was isolated as a yellow lyophilized powder (0.190 g, 10%). ¹H NMR (400 MHz, DMSO-d₆) δ ppm 1.81 (s, 6H) 2.44 (s, 3H) 2.75 (s, 3H) 7.09-7.16 (m, 3H) 7.19-7.27 (m, 2H) 7.41 (d, J=8.84 Hz, 1H) 8.36 (d, J=8.84 Hz, 1H).

### Compound 116: 3-hydroxy-8-isopropyl-2-(2-phenylpropan-2-yl)quinoline-4-carboxylic acid

Following the procedure described for the preparation of Compound 114, intermediate 2, 3-methyl-2-oxo-3-phenylbutyl acetate (0.80 g, 3.6 mmol, 0.7 equiv), was treated with intermediate 5, 7-isopropylindoline-2,3-dione (1.0 g, 5.29 mmol, 1.0 equiv) and 10.0 N aqueous sodium hydroxide solution (4.8 mL, 47.6 mmol, 9.0 equiv). The desired product was isolated as a yellow lyophilized powder (0.068 g, 4%). ¹H NMR (400 MHz, DMSO-d₆) δ ppm 1.39 (d, J=7.07 Hz, 6H) 1.81 (s, 6H) 3.56-4.87 (h, J=7.07 Hz, 1H) 7.04-7.18 (m, 3H) 7.19-7.28 (m, 2H) 7.47 (d, J=8.34 1H) 7.53 (t, J=8.34 1H) 8.41 (d, J=8.34 Hz, 1H).

### Compound 117: 3-hydroxy-2-(2-phenylpropan-2-yl)-8-(trifluoromethyl)quinoline-4-carboxylic acid

Following the procedure described for the preparation of Compound 114, intermediate 2, 3-methyl-2-oxo-3-phenylbutyl acetate (2.01 g, 9.07 mmol, 1.3 equiv), was treated with intermediate 6, 7-(trifluoromethyl)indoline-2,3-dione (1.5 g, 6.98 mmol, 1.0 equiv) and 10.0 N aqueous sodium hydroxide solution (6.2 mL, 62.8 mmol, 9.0 equiv). The desired product was isolated as a yellow lyophilized powder (0.486 g, 19%). ¹H NMR (400 MHz, DMSO-d₆) δ ppm 1.78 (s, 6H) 6.96-7.19 (m, 3H) 7.19-7.30 (m, 2H) 7.69 (t, J=8.08 1H) 7.95 (d, J=6.82 Hz, 1H) 8.98 (d, J=8.08 Hz, 1H).

### Compound 118: 2-(2-(4-Chlorophenyl)propan-2-yl)-3-hydroxy-8-isopropylquinoline-4-carboxylic acid

Following the procedure described for the preparation of Compound 114, intermediate 5, 7-isopropylindoline-2,3-dione (74 mg, 0.39 mmol) was treated with 3-(4-chlorophenyl)-3-methyl-2-oxobutyl acetate (intermediate 56, 99 mg, 0.39 mmol) to yield the desired product (9.8 mg, 6.6%) as a yellow solid. ¹H NMR (400 MHz, MeOH-D₄) δ ppm 1.63 (d, J=7.1 Hz, 6H), 2.04-2.10 (s, 6H), 4.61 (sept, J=7.1 Hz, 1H), 7.37 (d, J=8.6 Hz, 2H), 7.43 (d, J=8.6 Hz, 2H), 7.66-7.76 (m, 2H), 8.82 (dd, J=8.6, 1.5 Hz, 1H).

### Compound 119: 2-(2-(4-Chlorophenyl)propan-2-yl)-3-hydroxy-8-(trifluoromethyl)quinoline-4-carboxylic acid

Following the procedure described for the preparation of Compound 114, intermediate 6, 7-(trifluoromethyl)indoline-2,3-dione (85 mg, 0.39 mmol) was treated with 3-(4-chlorophenyl)-3-methyl-2-oxobutyl acetate (intermediate 56, 99 mg, 0.39 mmol) to yield the desired product (15.0 mg, 9.4%) as a yellow solid. ¹H NMR (400 MHz, MeOH-D₄) δ ppm 2.01-2.10 (s, 6H), 7.36 (d, J=9.0 Hz, 2H), 7.43 (d, J=9.0 Hz, 2H), 7.86 (dd, J=8.6, 7.7 Hz, 1H), 8.15 (d, J=7.7 Hz, 1H), 9.25 (d, J=8.6 Hz, 1H).

### Compound 120: 2-(2-(4-Chlorophenyl)propan-2-yl)-3-hydroxy-7,8,9,10-tetrahydrobenzo[h]quinoline-4-carboxylic acid

Following the procedure described for the preparation of Compound 114, intermediate 3 (80 mg, 0.39 mmol) was treated with 3-(4-chlorophenyl)-3-methyl-2-oxobutyl acetate (intermediate 56, 99 mg, 0.39 mmol) to yield the desired product (6.2 mg, 4.0%) as a yellow solid. ¹H NMR (400 MHz, MeOH-D₄) δ ppm 2.03-2.07 (s, 6H), 2.08-2.24 (m, 4H), 3.09-3.17 (m, 2H), 3.57-3.62 (m, 2H), 7.36 (d, J=9.0 Hz, 2H), 7.42 (d, J=9.0 Hz, 2H), 7.49 (d, J=9.0 Hz, 1H), 8.77 (d, J=9.0 Hz, 1H).

### Compound 121: 2-(2-(4-Chlorophenyl)propan-2-yl)-3-hydroxy-7,8-dimethylquinoline-4-carboxylic acid

Following the procedure described for the preparation of Compound 114, intermediate 4, 6,7-dimethylindoline-2,3-dione (70 mg, 0.39 mmol) was treated with 3-(4-chlorophenyl)-3-methyl-2-oxobutyl acetate (intermediate 56, 99 mg, 0.39 mmol) to yield the desired product (11.9 mg, 8.3%) as a yellow solid. ¹H NMR (400 MHz, MeOH-D₄) δ ppm 2.04-2.09 (s, 6H), 2.66-2.72 (s, 3H), 2.98-3.05 (s, 3H), 7.36 (d, J=8.5 Hz, 2H), 7.42 (d, J=8.5 Hz, 2H), 7.57 (d, J=8.5 Hz, 1H), 8.78 (d, J=8.5 Hz, 1H).

### Compound 122: 2-(2-(4-chlorophenyl)propan-2-yl)-8-(1,1,1,3,3,3-hexafluoro-2-hydroxypropan-2-yl)-3-hydroxyquinoline-4-carboxylic acid

Following the procedure described for the preparation of Compound 114, intermediate 16, 7-(1,1,1,3,3,3-hexafluoro-2-hydroxypropan-2-yl)indoline-2,3-dione (130 mg, 0.39 mmol) was treated with 3-(4-chlorophenyl)-3-methyl-2-oxobutyl acetate (intermediate 56, 99 mg, 0.39 mmol) to yield the desired product (14.0 mg, 7.1%) as a yellow solid. ¹H NMR (400 MHz, MeOH-D₄) δ ppm 2.04-2.10 (m, 3H,) 2.24-2.29 (m, 3H), 7.36 (d, J=8.3 Hz, 2H), 7.48 (d, J=8.3 Hz, 2H), 7.96 (dd, J=9.3, 8.3 Hz, 1H), 8.16 (d, J=8.3 Hz, 1H), 9.19 (d, J=9.3 Hz, 1H).

### Compound 123: 3-hydroxy-2-(1-phenylethyl)-8-(trifluoromethyl)quinoline-4-carboxylic acid

Following the procedure described for the preparation of Compound 114, intermediate 40, 2-oxo-3-phenylbutyl acetate (0.922 g, 4.47 mmol, 1.3 equiv), was treated with intermediate 6, 7-(trifluoromethyl)indoline-2,3-dione (0.740 g, 3.44 mmol, 1.0 equiv) and 10.0 N sodium hydroxide (2.8 mL, 27.5 mmol, 8.0 equiv) to yield the desired product as an orange lyophilized powder (0.450 g, 36%). ¹H NMR (400 MHz, DMSO-d₆) δ ppm 1.68 (d, J=7.07 Hz, 3H) 4.87 (q, J=7.07 Hz, 1H) 6.90-7.41 (m, 5H) 7.69 (t, J=6.82 Hz, 1H) 7.97 (d, J=6.82 Hz, 1H) 8.84 (d, J=8.34 Hz, 1H).

### Compound 124: 2-(1-(4-chlorophenyl)ethyl)-3-hydroxy-7,8,9,10-tetrahydrobenzo[h]quinoline-4-carboxylic acid

Following the procedure described by Cragoe et al. (J. Org. Chem., 1953, 18, 561), to a mixture of intermediate 3 (0.16 g, 0.8 mmol) in 0.5 mL EtOH and 1 mL aq. 6 M KOH at 100° C. was added warm 3-(4-chlorophenyl)-2-oxobutyl acetate (intermediate 53, 0.21 g, 0.9 mmol) in 0.5 mL EtOH in small portions over 0.5 h period. After the addition was completed, the reaction mixture was refluxed for additional time until LC/MS indicated the reaction was complete. After removal of the solvent, the resulting yellow gum was dissolved in 1 mL DMSO. HPLC of the resulting DMSO solution under basic conditions (triethylamine) yielded the desired product as the triethylammonium salt. The salt was then dissolved in 1 mL acetonitrile and acidified with concentrated hydrochloric acid to pH˜1 at 0° C. Water (20 mL) was added, and the resulting suspension was stirred vigorously at 0° C. for 1 h. The yellow solid was collected via filtration, washed with water, and dried under vacuum to yield the desired product (17 mg, 5.6%). ¹H NMR (400 MHz, MeOH-D₄) δ ppm 1.69 (d, J=7.0 Hz, 3H), 1.80-1.97 (m, 4H), 2.79-2.88 (m, 2H), 3.25-3.35 (m, 2H), 4.81 (q, J=7.0 Hz, 1H), 7.12 (d, J=9.6 Hz, 1H), 7.18 (d, J=8.5 Hz, 2H), 7.34 (d, J=8.5 Hz, 2H), 8.80 (d, J=9.6 Hz, 1H).

### Compound 125: 3-Hydroxy-2-(1-phenylethyl)-7,8,9,10-tetrahydrobenzo[h]quinoline-4-carboxylic acid

To a 25 mL round bottom flask equipped with a condenser was added intermediate 3, 6,7,8,9-tetrahydro-1H-benzo[g]indole-2,3-dione (0.176 g, 0.7 mmol, 1.0 equiv) and 4 mL ethanol. To this solution was added 10.0 N aqueous sodium hydroxide solution (0.63 mL, 6.3 mmol, 9.0 equiv) and the mixture was heated to reflux in an oil bath. To this solution was added a solution of intermediate 40, 2-oxo-3-phenylbutyl acetate (0.187 g, 0.91 mmol, 1.3 equiv) in 1.0 mL ethanol over 60 minutes. The resulting mixture was allowed to stir at reflux for an additional 3 hours. Upon cooling to room temperature, and the ethanol removed under reduced pressure. The mixture was acidified to pH 1 with 1M HCl and poured into water. The crude solid obtained was purified by reverse-phase HPLC (water/acetonitrile/0.1% triethyl amine) and lyophilized to give the desired product as a yellow lyophilized powder (0.102 g, 42%). ¹H NMR (400 MHz, DMSO-d₆) δ ppm 1.68 (d, J=6.8 Hz, 3H) 1.75-1.96 (m, 4H) 2.84 (t, J=6.7 Hz, 2H) 3.30 (t, J=6.8 Hz, 1H) 4.71-4.93 (m, 1H) 7.14 (t, J=8.0 Hz, 1H) 7.20-7.29 (m, 3H) 7.33 (d, J=7.6 Hz, 2H) 8.14-8.38 (m, 1H).

### Compound 126: 3-Hydroxy-2-(1-phenylpropyl)-8-(trifluoromethyl)quinoline-4-carboxylic acid

Following the procedure described for the preparation of Compound 114, intermediate 6, 7-(trifluoromethyl)indoline-2,3-dione (200 mg, 0.93 mmol) was treated with 1-hydroxy-3-phenylpentan-2-one (intermediate 57, 180 mg, 1.00 mmol) to yield the desired product (100.7 mg, 28.7%) as a light yellow solid. ¹H NMR (400 MHz, MeOH-D₄) δ ppm 1.11 (t, J=7.5 Hz, 3H), 2.30-2.42 (m, 1H), 2.64-2.77 (m, 1H), 4.85 (t, J=8.2 Hz, 1H), 7.31-7.38 (m, 1H), 7.40-7.48 (m, 2H), 7.63 (d, 2H), 7.85 (dd, J=8.3, 7.6 Hz, 1H), 8.14 (d, J=7.6 Hz, 1H), 9.29 (d, J=8.3 Hz, 1H).

### Compound 127: 3-Hydroxy-8-isopropyl-2-(1-phenylpropyl)quinoline-4-carboxylic acid

Following the procedure described for the preparation of Compound 114, intermediate 5, 7-isopropylindoline-2,3-dione (124.7 mg, 0.66 mmol) was treated with 1-hydroxy-3-phenylpentan-2-one (intermediate 57, 130 mg, 0.73 mmol) to yield the desired product (30.8 mg, 13.4%) as a yellow solid. ¹H NMR (400 MHz, MeOH-D₄) δ ppm 1.14 (t, J=7.5 Hz, 3H), 1.63 (d, J=6.7 Hz, 6H), 2.28-2.45 (m, 1H), 2.64-2.81 (m, 1H), 4.54-4.70 (sept, J=6.7 Hz, 1H), 4.86 (t, J=7.5 Hz, 1H), 7.32-7.38 (m, 1H), 7.46 (dd, J=6.7, 6.7 Hz, 2H), 7.61 (d, J=7.6 Hz, 2H), 7.64-7.77 (m, 2H), 8.86 (d, J=8.4 Hz, 1H).

### Compound 128: 3-Hydroxy-7,8-dimethyl-2-(1-phenylpropyl)quinoline-4-carboxylic acid

Following the procedure described for the preparation of Compound 114, intermediate 4, 6,7-dimethylindoline-2,3-dione (130.0 mg, 0.66 mmol) with 1-hydroxy-3-phenylpentan-2-one (intermediate 57, 130 mg, 0.74 mmol) to yield the desired product (39.0 mg, 15.7%) as a white solid. ¹H NMR (400 MHz, MeOH-D₄) δ ppm 1.14 (t, J=7.7 Hz, 3H), 2.31-2.47 (m, 1H), 2.63-2.78 (m, 1H), 2.68 (s, 3H), 3.00 (s, 3H), 4.83 (t, J=7.7 Hz, 1H), 7.31-7.33 (m, 1H), 7.42-7.44 (m, 2H), 7.55 (d. J=8.6 Hz, 1H), 7.62 (d, J=8.2 Hz, 2H), 8.75 (d, J=9.0 Hz, 1H).

### Compound 129: 3-Hydroxy-2-(2-methyl-1-phenylpropyl)-8-(trifluoromethyl)quinoline-4-carboxylic acid

Following the procedure described for the preparation of Compound 114, intermediate 6, 7-(trifluoromethyl)indoline-2,3-dione (150 mg, 0.70 mmol) was treated with 1-hydroxy-4-methyl-3-phenylpentan-2-one (intermediate 59, 147 mg, 0.76 mmol) to yield the desired product (43.7 mg, 16.0%) as a white solid. ¹H NMR (400 MHz, MeOH-D₄) δ ppm 1.04 (d, J=6.5 Hz, 3H), 1.12 (d, J=6.5 Hz, 3H), 3.11-3.25 (m, 1H), 4.57 (d, J=10.6 Hz, 1H), 7.28-7.34 (m, 1H), 7.41 (dd, J=7.2, 7.1 Hz, 2H), 7.69 (d, J=7.7 Hz, 1H), 7.73 (d, J=8.3 Hz, 2H), 7.99 (d, J=7.1 Hz, 1H), 9.75 (d, J=8.9 Hz, 1H).

### Compound 130: 3-Hydroxy-8-isopropyl-2-(2-methyl-1-phenylpropyl)quinoline-4-carboxylic acid

Following the procedure described for the preparation of Compound 114, intermediate 5, 7-isopropylindoline-2,3-dione (119 mg, 0.63 mmol) was treated with 1-hydroxy-4-methyl-3-phenylpentan-2-one (intermediate 59, 130 mg, 0.68 mmol) to yield the desired product (8.1 mg, 3.5%) as a yellow solid. ¹H NMR (400 MHz, MeOH-D₄) δ ppm 1.08 (d, J=6.5 Hz, 3H), 1.15 (d, J=6.5 Hz, 3H), 1.64 (d, J=7.1 Hz, 3H), 1.66 (d, J=7.1 Hz, 3H), 3.15-3.28 (m, 1H), 4.60 (d, J=10.5 Hz, 1H), 4.60-4.70 (m, 1H), 7.31-7.38 (m, 1H), 7.40-7.47 (m, 2H), 7.64-7.74 (m, 4H), 8.80-8.88 (m, 1H).

### Compound 131: 3-Hydroxy-7,8-dimethyl-2-(2-methyl-1-phenylpropyl)quinoline-4-carboxylic acid

Following the procedure described for the preparation of Compound 114, intermediate 4, 6,7-dimethylindoline-2,3-dione (105 mg, 0.60 mmol) was treated with 1-hydroxy-4-methyl-3-phenylpentan-2-one (intermediate 59, 126 mg, 0.66 mmol) to yield the desired product (12.5 mg, 6.0%) as a yellow solid. ¹H NMR (400 MHz, MeOH-D₄) δ ppm 1.08 (d, J=6.7 Hz, 3H), 1.15 (d, J=6.7 Hz, 3H), 2.69 (s, 3H), 3.04 (s, 3H), 3.15-3.28 (m, 1H), 4.57 (d, J=10.8 Hz, 1H), 7.30-7.36 (m, 1H), 7.40-7.46 (m, 2H), 7.54 (d, J=8.9 Hz, 1H), 7.68-7.74 (m, 2H), 8.76 (d, J=8.9 Hz, 1H).

### Compound 132: 3-Hydroxy-2-(1-Phenylpropan-2-yl)-8-(trifluoromethyl)quinoline-4-carboxylic acid

Following the procedure described for the preparation of Compound 114, intermediate 6, 7-(trifluoromethyl)indoline-2,3-dione (150 mg, 0.70 mmol) was treated with 1-hydroxy-3-methyl-4-phenylbutan-2-one (intermediate 60, 136 mg, 0.76 mmol) to yield the desired product (51.6 mg, 19.6%) as a yellow solid. ¹H NMR (400 MHz, MeOH-D₄) δ ppm 1.50 (s, 3H), 3.07 (dd, J=13.4, 7.4 Hz, 1H), 3.57 (dd, J=13.4, 7.4 Hz, 1H), 4.10-4.23 (m, 1H), 7.25-7.42 (m, 5H), 7.80 (dd, J=8.5, 8.0 Hz, 1H), 8.09 (d, J=7.4 Hz, 1H), 9.35 (d, J=8.5 Hz, 1H).

### Compound 133: 3-Hydroxy-8-isopropyl-2-(1-phenylpropan-2-yl)quinoline-4-carboxylic acid

Following the procedure described for the preparation of Compound 114, intermediate 5, 7-isopropylindoline-2,3-dione (130 mg, 0.70 mmol) was treated with 1-hydroxy-3-methyl-4-phenylbutan-2-one (intermediate 60, 136 mg, 0.76 mmol) to yield the desired product (14.0 mg, 5.7%) as a yellow solid. ¹H NMR (400 MHz, MeOH-D₄) δ ppm 1.54-1.62 (m, 9H), 3.11 (dd, J=13.4, 7.6 Hz, 1H), 3.54-3.59 (dd, J=13.4, 6.9 Hz, 1H), 4.12-4.22 (m, 1H), 4.52-4.62 (m, 1H), 7.27-7.34 (m, 1H), 7.34-7.43 (m, 4H), 7.62-7.72 (m, 2H), 8.82 (dd, dd, J=8.3, 1.8 Hz, 1H).

### Compound 134: 3-Hydroxy-7,8-dimethyl-2-(1-phenylpropan-2-yl)quinoline-4-carboxylic acid

Following the procedure described for the preparation of Compound 114, intermediate 4, 6,7-dimethylindoline-2,3-dione (126 mg, 0.70 mmol) was treated with 1-hydroxy-3-methyl-4-phenylbutan-2-one (intermediate 60, 136 mg, 0.76 mmol) to yield the desired product (13.0 mg, 5.5%) as a yellow solid. ¹H NMR (400 MHz, MeOH-D₄) δ ppm 1.55 (d, J=6.8 Hz, 3H), 2.67 (s, 3H), 2.96 (s, 3H), 3.09 (dd, J=12.8, 7.2 Hz, 1H), 3.59 (dd, J=12.8, 7.2 Hz, 1H), 4.06-4.20 (m, 1H), 7.27-7.35 (m, 1H), 7.35-7.43 (m, 4H), 7.55 (d, J=8.8 Hz, 1H), 8.73 (d, J=8.8 Hz, 1H).

### Compound 135: 3-Hydroxy-2-(2-phenylpropyl)-8-(trifluoromethyl)quinoline-4-carboxylic acid

Following the procedure described for the preparation of Compound 114, intermediate 6, 7-(trifluoromethyl)indoline-2,3-dione (150 mg, 0.70 mmol) was treated with 1-hydroxy-4-phenylpentan-2-one (intermediate 61, 136 mg, 0.76 mmol) to yield the desired product (46.1 mg, 17.6%) as a white solid. ¹H NMR (400 MHz, MeOH-D₄) δ ppm 1.56 (d, J=7.1 Hz, 3H), 3.47 (dd, J=14.7, 8.4 Hz, 1H), 3.64 (dd, J=14.7, 6.7 Hz, 1H), 3.85-3.96 (m, 1H), 7.28-7.35 (m, 1H), 7.43 (dd, J=7.6, 7.6 Hz, 2H), 7.50 (d, J=7.6 Hz, 2H), 7.82 (dd, J=8.4, 7.6 Hz, 1H), 8.10 (d, J=7.6 Hz, 1H), 9.26 (d, J=8.4 Hz, 1H).

### Compound 136: 3-Hydroxy-8-isopropyl-2-(2-phenylpropyl)quinoline-4-carboxylic acid

Following the procedure described for the preparation of Compound 114, intermediate 5, 7-isopropylindoline-2,3-dione (130 mg, 0.70 mmol) was treated with 1-hydroxy-4-phenylpentan-2-one (intermediate 61, 136 mg, 0.76 mmol) to yield the desired product (22.4 mg, 9.2%) as a yellow solid. ¹H NMR (400 MHz, MeOH-D₄) δ ppm 1.32 (d, J=7.0 Hz, 3H), 1.36 (d, J=6.5 Hz, 3H), 1.42 (d, J=7.0 Hz, 3H), 3.37-3.40 (m, 1H), 3.41-3.50 (m, 1H), 3.63-3.72 (m, 1H), 4.24-4.36 (m, 1H), 7.13-7.19 (m, 1H), 7.22-7.35 (m, 4H), 7.42-7.55 (m, 2H), 8.70 (d, J=8.1 Hz, 1H).

### Compound 137: 3-Hydroxy-7,8-dimethyl-2-(2-phenylpropyl)quinoline-4-carboxylic acid

Following the procedure described for the preparation of Compound 114, intermediate 4, 6,7-dimethylindoline-2,3-dione (126 mg, 0.70 mmol) was treated with 1-hydroxy-4-phenylpentan-2-one (intermediate 61, 136 mg, 0.76 mmol) to yield the desired product (27.7 mg, 11.8%) as a yellow solid. ¹H NMR (400 MHz, MeOH-D₄) δ ppm 1.61 (d, J=7.2 Hz, 3H), 2.65 (s, 3H), 2.82 (s, 3H), 3.57-3.66 (m, 2H), 3.76-3.89 (m, 1H), 7.29-7.37 (m, 1H), 7.40-7.50 (m, 4H), 7.59 (d, J=8.6 Hz, 1H), 8.80-8.95 (m, 1H).

### Compound 138: 2-(4-Chlorobenzyl)-3-[(morpholin-4-ylcarbonyl)oxy]-7,8,9,10-tetrahydrobenzo[h]quinoline-4-carboxylic acid

A mixture of 2-(4-chlorobenzyl)-3-hydroxy-7,8,9,10-tetrahydrobenzo[h]quinoline-4-carboxylic acid (0.124 g, 0.338 mmol) (prepared as described in J. Med. Chem. 2007, 50, 40), 4-morpholinecarbonyl chloride (42 □L, 0.37 mmol), triethylamine (52 □L, 0.37 mmol), and 1.0 mL THF/1.0 mL pyridine was stirred at 25° C. for 16 h. Concentration of the reaction mixture gave an oily residue. HPLC purification of the residue under basic conditions afforded a white solid, which was acidified at 0° C. with 1N aq. HCl to pH˜1. The precipitate was collected by filtration, washed with water, and dried under vacuum to yield the product (12.5 mg, 7.7%) as a white solid. ¹H NMR (400 MHz, MeOD-D₆): δ 1.85-1.98 (m, 4H), 2.87-2.97 (m, 2H), 3.23-3.30 (m, 2H), 3.48-3.67 (m, 4H), 3.69-3.79 (m, 4H), 4.21-4.29 (m, 2H), 7.18-7.28 (m, 4H), 7.71-7.80 (m, 2H). HRMS (ESI+) calcd for C₂₆H₂₅ClN₂O₅ (MH⁺) 481.15248, found 481.1521.

### Biological Test

### Biacore P-Selectin/PSGL-1 Inhibition Assay

Surface plasmon resonance assays were performed on a Biacore 3000 instrument (Biacore Inc. Piscataway, N.J.) at 25° C. at a flow rate of 30 μL/minute and each assay consisted of a 60-second equilibration, a 60-μL sample injection (kinject), and a 300-second dissociation.

A purified, monomeric, truncated form of human PSGL-1, “19ek”, that contained all the necessary P-selectin binding determinants (see Goetz, et al., J. Cell Biol., 1997, 137: 509-519; and Sako, et al., Cell, 1995, 83: 323-331) was biotinylated via amine chemistry (Sulfo-NHS-LC-Biotin, Peirce) at a unique C-terminal lysine residue (see Somers, et al., Cell, 2000, 103: 467-479) and immobilized on a Biacore SA sensor chip (Biacore Inc.), using an HBS-EP buffer (Biacore Inc.), and the target 600-700 RU. The coated chip was re-equilibrated with an HBS-P buffer (Biacore Inc.) to which 1 mM CaCl₂ and 1 mM MgCl₂ (both from Fisher) were added to ensure sufficient calcium for the calcium-dependent interaction between the receptor and the ligand.

Test compounds were incubated for 1 hour in a 1.1× Biacore assay buffer. Each solution was centrifuged through a 0.2 □m filter, using a 96-well plate format (Millipore). Glycyrrhizin tri-sodium salt (TCI) was prepared as a positive control in parallel with the test compounds, in the same manner described above. Glycyrrhizin, a demonstrated antagonist of P-selectin (see Patton, J. T., GlycoTech Corporation, written communication, May 2000), has been shown to inhibit the P-selectin/PSGL-1 interaction with an IC₅₀ of 1 mM in this assay.

A soluble recombinant truncated form of human P-selectin, P-LE, comprised of the lectin and epidermal growth factor-like (EGF) domains expressed in CHO cells (see Somers, et al., Cell, 2000, 103: 467-479) was added to each filtered test compound solution. Final concentrations of reagents were 500 nM P.LE, 250 or 500 μM test compound (depending on structure) or 1 mM glycyrrhizin, 10% DMSO, and 1× Biacore buffer (100 mM HEPES, 150 mM NaCl, 1 mM CaCl₂, and 1 mM MgCl₂ (all reagents from Fisher)), with a pH of 7.4. Compounds active at 250 μM were titrated to further define activity. Test samples were supplied to the Biacore instrument in a 96-well plate.

The Biacore raw data file was exported as a text file to an Excel spreadsheet, where the buffer blanks bracketing the samples were averaged for each Biacore instrument flow cell (Fc), and subtracted from the averaged uninhibited P.LE samples and from all the other samples. The reference signal from Fcl (uncoated) was then subtracted from its corresponding active (coated) signal for each injection, a process known as double referencing (see Myszka, J. Mol. Recognit., 1999, 12(5): 279-284). The percent inhibition of binding was calculated by dividing the reference-subtracted inhibited signal by the reference-subtracted uninhibited signal, subtracting this value from 1, and multiplying the resulting value by 100. The replicate percent inhibition values were averaged and expressed as the mean ±standard deviation. The inter-experiment standard deviation of calculated percent inhibitions in the Biacore assay was ±5.

Assay results for representative compounds according to the invention are included in Table 1 below.

TABLE 1

| | | | % inhibition |
|---|---|---|---|
| Compound | Name | Structure | at 250 uM |
| 1 | 2-(1-(4-chlorophenyl)cyclopropyl)-3-hydroxy-8-(trifluoromethoxy)quinoline-4-carboxylic acid | 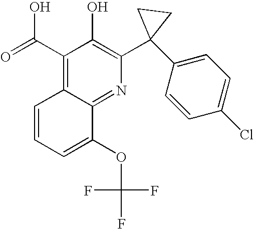 | 37 |
| 2 | 2-(1-(4-chlorophenyl)cyclopropyl)-8-ethyl-3-hydroxyquinoline-4-carboxylic acid | 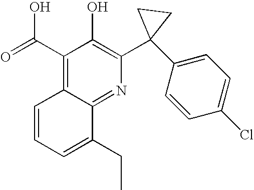 | 67 |
| 3 | 8-sec-butyl-2-(1-(4-chlorophenyl)cyclopropyl)-3-hydroxyquinoline-4-carboxylic acid | 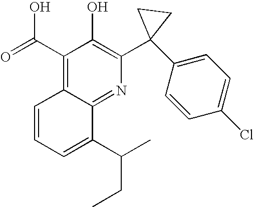 | 18 |
| 4 | 8-tert-butyl-2-(1-(4-chlorophenyl)cyclopropyl)-3-hydroxyquinoline-4-carboxylic acid | 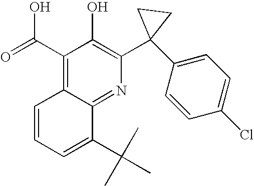 | ≦10 |
| 5 | 8-chloro-2-(1-(4-chlorophenyl)cyclopropyl)-3-hydroxyquinoline-4-carboxylic acid | 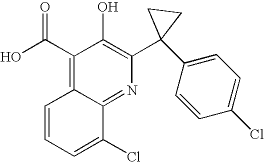 | 93 |
| 6 | 2-(1-(4-chlorophenyl)cyclopropyl)-3-hydroxy-8-phenylquinoline-4-carboxylic acid | 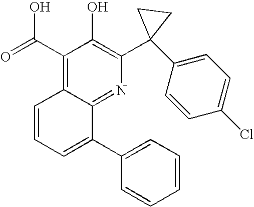 | 98 |
| 7 | 2-(1-(4-chlorophenyl)cyclopropyl)-8-fluoro-3-hydroxyquinoline-4-carboxylic acid | 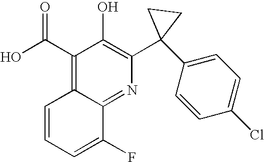 | ≦10 |
| 8 | 2-(1-(4-chlorophenyl)cyclopropyl)-3-hydroxyquinoline-4-carboxylic acid | 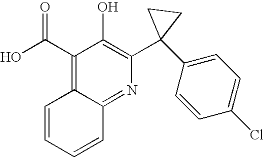 | ≦10 |
| 9 | 8-bromo-2-(1-(4-chlorophenyl)cyclopropyl)-3-hydroxyquinoline-4-carboxylic acid | 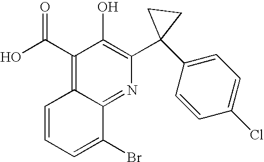 | 46 |
| 10 | 2-(1-4-chlorophenyl)cyclopropyl)-3-hydroxy-6,8-dimethylquinoline-4-carboxylic acid | 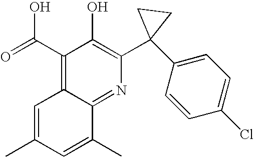 | 47 |
| 11 | 2-(1-(4-chlorophenyl)cyclopropyl)-3-hydroxy-8-methylquinoline-4-carboxylic acid | 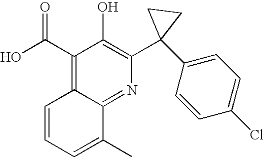 | 43 |
| 12 | 2-(1-(4-chlorophenyl)cyclopropyl)-7-ethyl-3-hydroxyquinoline-4-carboxylic acid | 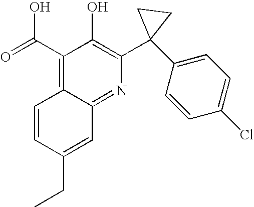 | 13 |
| 13 | 2-(1-(4-chlorophenyl)cyclopropyl)-3-hydroxy-7-methylquinoline-4-carboxylic acid | 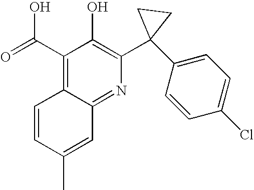 | ≦10 |
| 14 | 8-ethyl-3-hydroxy-2-(1-phenylcyclopropyl)quinoline-4-carboxylic acid | 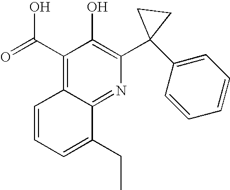 | 23 |
| 15 | 8-sec-butyl-3-hydroxy-2-(1-phenylcyclopropyl)quinoline-4-carboxylic acid | 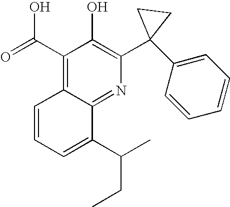 | 63 |
| 16 | 7-chloro-2-(1-(4-chlorophenyl)cyclopropyl)-3-hydroxyquinoline-4-carboxylic acid | 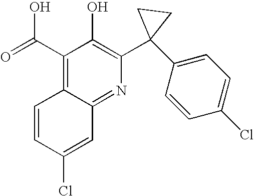 | 28 |
| 17 | 2-(1-(4-chlorophenyl)cyclopropyl)-6-fluoro-3-hydroxyquinoline-4-carboxylic acid | 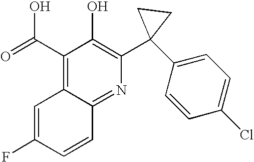 | ≦10 |
| 18 | 6-bromo-2-(1-(4-chlorophenyl)cyclopropyl)-3-hydroxyquinoline-4-carboxylic acid | 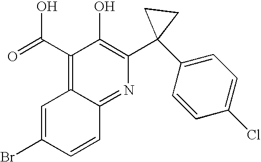 | 28 |
| 19 | 2-(1-(4-chlorophenyl)cyclopropyl)-3-hydroxy-6-methylquinoline-4-carboxylic acid | 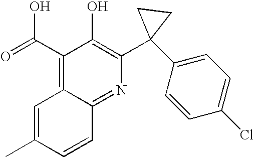 | ≦10 |
| 20 | 2-(1-(4-chlorophenyl)cyclopropyl)-3-hydroxy-6-methoxyquinoline-4-carboxylic acid | 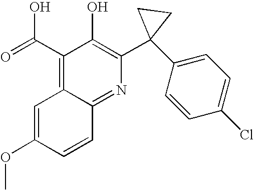 | ≦10 |
| 21 | 2-(1-(4-chlorophenyl)cyclopropyl)-3-hydroxy-6-(trifluoromethoxy)quinoline-4-carboxylic acid | 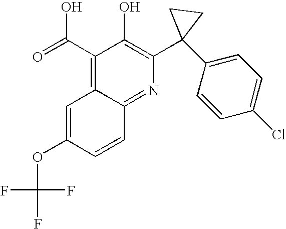 | 28 |
| 22 | 6-chloro-2-(1-(4-chlorophenyl)cyclopropyl)-3-hydroxyquinoline-4-carboxylic acid | 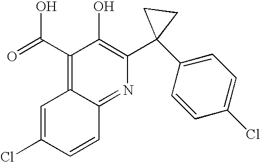 | 26 |
| 23 | 2-(1-(4-chlorophenyl)cyclopropyl)-3,6-dihydroxyquinoline-4-carboxylicacid | 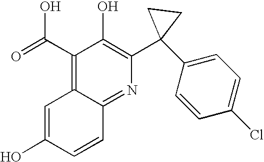 | ≦10 |
| 24 | 2-(1-(4-chlorophenyl)cyclopropyl)-3-hydroxy-6-(trifluoromethyl)quinoline-4-carboxylic acid | 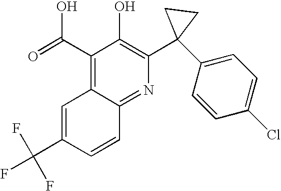 | 34 |
| 25 | 2-(1-(4-chlorophenyl)cyclopropyl)-3-hydroxy-6-(isopropylquinoline-4-carboxylic acid | 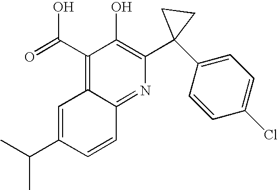 | 14 |
| 26 | 7-chloro-3-hydroxy-2-(1-phenylcyclopropyl)quinoline-4-carboxylic acid | 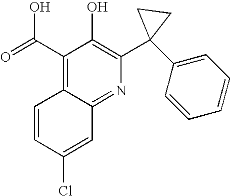 | ≦10 |
| 27 | 6-ethyl-3-hydroxy-2-(1-phenylcyclopropyl)quinoline-4-carboxylic acid | 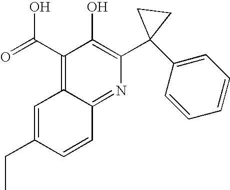 | ≦10 |
| 28 | 7-ethyl-3-hydroxy-2-(1-phenylcyclopropyl)quinoline-4-carboxylic acid | 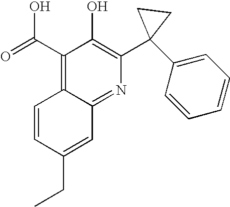 | ≦10 |
| 29 | 3-hydroxy-2-(1-phenylcyclopropyl)-6-(trifluoromethoxy)quinoline-4-carboxylic acid | 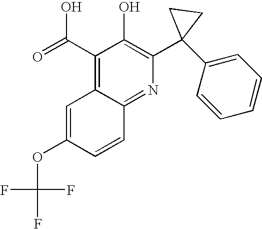 | 10 |
| 30 | 6-chloro-3-hydroxy-2-(1-phenylcyclopropyl)quinoline-4-carboxylic acid | 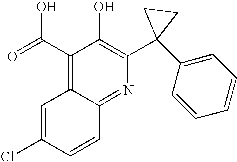 | ≦10 |
| 31 | 3-hydroxy-8-methyl-2-(1-phenylcyclopropyl)quinoline-4-carboxylic acid | 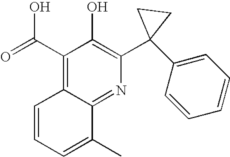 | ≦10 |
| 32 | 3-hydroxy-2-(1-phenylcyclopropyl)-6-(trifluoromethyl)quinoline-4-carboxylic acid | 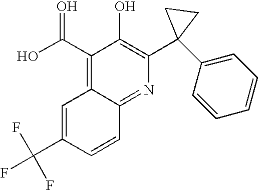 | ≦10 |
| 33 | 3-hydroxy-6-methyl-2-(1-phenylcyclopropyl)quinoline-4-carboxylic acid | 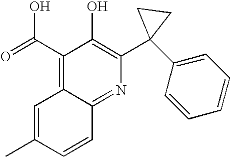 | ≦10 |
| 34 | 3-hydroxy-2-(1-phenylcyclopropyl)-8-(trifluoromethyl)quinoline-4-carboxylic acid | 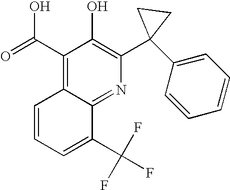 | 44 |
| 35 | 3-hydroxy-2-(1-phenylcyclopropyl)-8-(thiophen-3-yl)quinoline-4-carboxylic acid | 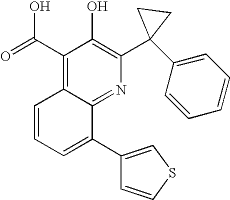 | 70 |
| 36 | 2-(1-(4-chlorophenyl)cyclopropyl)-3-hydroxy-7,8,9,10-tetrahydrobenzo[h]quinoline-4-carboxylic acid | 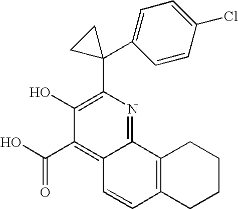 | 67 |
| 37 | 2-(1-(4-chlorophenyl)cyclopropyl)-3-hydroxy-8-(thiophen-3-yl)quinoline-4-carboxylic acid | 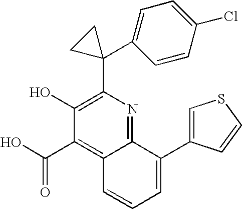 | 78 |
| 38 | 2-(1-(4-chlorophenyl)cyclopropyl)-3-hydroxy-8-(trifluoromethyl)quinoline-4-carboxylic acid | 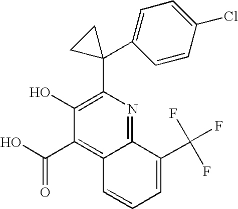 | 52 |
| 39 | 2-(1-(4-chlorophenyl)cyclopropyl)-3-hydroxy-8-isopropylquinoline-4-carboxylic acid | 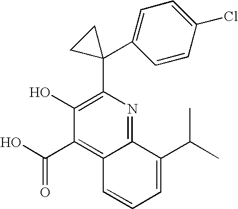 | 66 |
| 40 | 2-(1-(4-chlorophenyl)cyclopropyl)-3-hydroxy-7,8-dimethylquinoline-4-carboxylic acid | 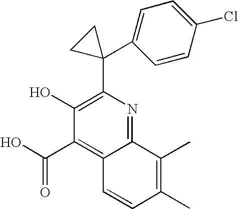 | 53 |
| 41 | 2-(1-(4-chlorophenyl)cyclopropyl)-8-(1,1,1,3,3,3-hexafluoro-2-hydroxypropan-2-yl)-3-hydroxyquinoline-4-carboxylic acid | 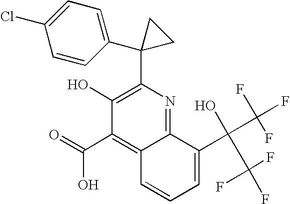 | ≦10 |
| 42 | 3-hydroxy-2-(1-phenylcyclopropyl)-7,8,9,10-tetrahydrobenzo[h]quinoline-4-carboxylic acid | 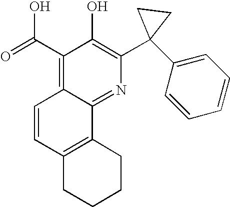 | 52 |
| 43 | 3-hydroxy-7,8-dimethyl-2-(1-phenylcyclopropyl)quinoline-4-carboxylic acid | 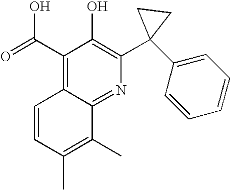 | 18 |
| 44 | 3-hydroxy-8-isopropyl-2-(1-phenylcyclopropyl)quinoline-4-carboxylic acid | 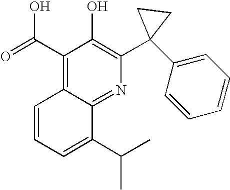 | 63 |
| 45 | 3-hydroxy-8-phenyl-2-(1-phenylcyclopropyl)quinoline-4-carboxylic acid | 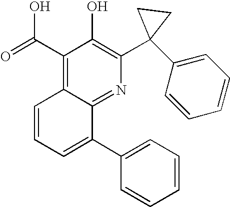 | 37 |
| 46 | 3-hydroxy-2-(1-phenylcyclopropyl)-8-(trifluoromethoxy)quinoline-4-carboxylic acid | 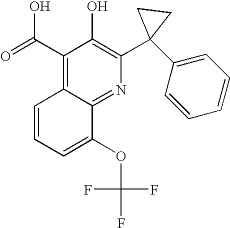 | ≦10 |
| 47 | 8-chloro-3-hydroxy-2-(1-phenylcyclopropyl)quinoline-4-carboxylic acid | 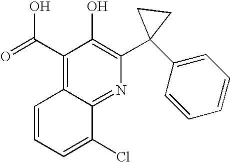 | ≦10 |
| 48 | 6-(1,1,1,3,3,3-hexafluoro-2-hydroxypropan-2-yl)-3-hydroxy-2-(1-phenylcyclopropyl)quinoline-4-carboxylic acid | 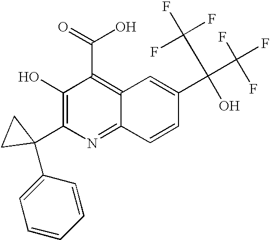 | ≦10 |
| 49 | 8-(1,1,1,3,3,3-hexafluoro-2-hydroxypropan-2-yl)-3-hydroxy-2-(1-phenylcyclopropyl)quinoline-4-carboxylic acid | 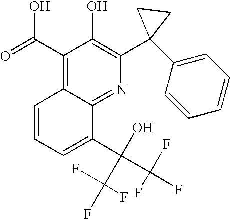 | 58 |
| 50 | 3-hydroxy-2-(1-(4-methoxyphenyl)cyclopropyl)-8-(trifluoromethyl)quinoline-4-carboxylic acid | 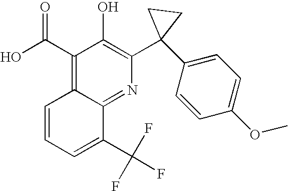 | 36 |
| 51 | 3-hydroxy-2-(1-(4-methoxyphenyl)cyclopropyl)-7,8,9,10-tetrahydrobenzo[h]quinoline-4-carboxylic acid | 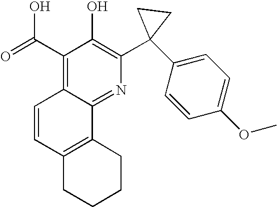 | 38 |
| 52 | 3-hydroxy-8-(trifluoromethyl)-2-(1-(4-(trifluoromethyl)phenyl)cyclopropyl)quinoline-4-carboxylicacid | 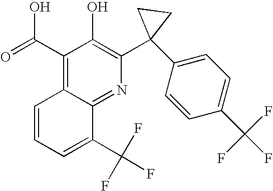 | 47 |
| 53 | 2-(1-(4-bromophenyl)cyclopropyl)-3-hydroxy-8-(trifluoromethyl)quinoline-4-carboxylic acid | 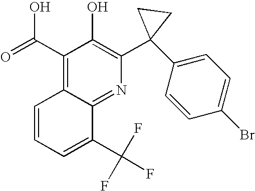 | 64 |
| 54 | 2-(1-(3-chlorophenyl)cyclopropyl)-3-hydroxy-8-(trifluoromethyl)quinoline-4-carboxylic acid | 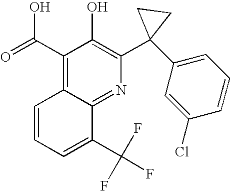 | 66 |
| 55 | 2-(1-(2-chlorophenyl)cyclopropyl)-3-hydroxy-8-(trifluoromethyl)quinoline-4-carboxylic acid | 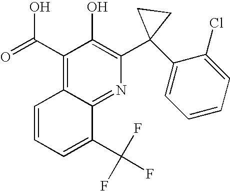 | 57 |
| 56 | 3-hydroxy-2-(1-(4-(trifluoromethoxy)phenyl)cyclopropyl)-8-(trifluoromethyl)quinoline-4-carboxylic acid | 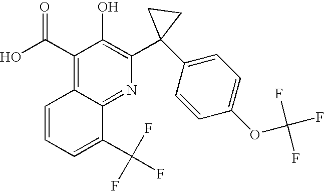 | 29 |
| 57 | 3-hydroxy-8-(trifluoromethyl)-2-(1-(3-(trifluoromethyl)phenyl)cyclopropyl)quinoline-4-carboxylicacid | 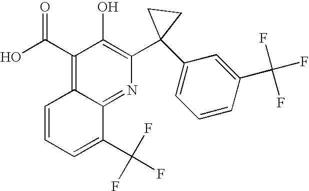 | 42 |
| 58 | 2-(1-(4-chlorophenyl)cyclobutyl)-3-hydroxy-8-(trifluoromethyl)quinoline-4-carboxylic acid | 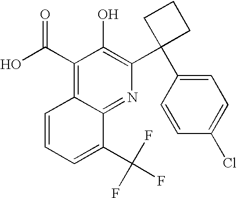 | ≦10 |
| 59 | 3-hydroxy-2-(1-(thiophen-3-yl)cyclopropyl)-8-(trifluoromethyl)quinoline-4-carboxylic acid | 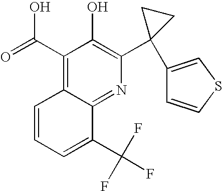 | ≦10 |
| 60 | 3-hydroxy-2-(1-(thiophen-2-yl)cyclopropyl)-8-(trifluoromethyl)quinoline-4-carboxylic acid | 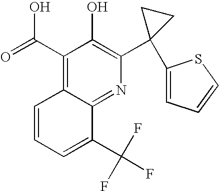 | 15 |
| 61 | 2-(1-(4-fluorophenyl)cyclopropyl)-3-hydroxy-8-(trifluoromethyl)quinoline-4-carboxylic acid | 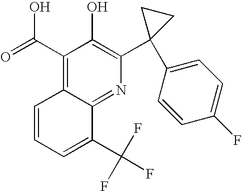 | 56 |
| 62 | 2-(1-(4-fluorophenyl)cyclopropyl)-3-hydroxy-8-isopropylquinoline-4-carboxylic acid | 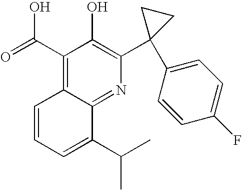 | 67 |
| 63 | 3-hydroxy-8-(trifluoromethyl)-2-(1-(2-(trifluoromethyl)phenyl)cyclopropyl)quinoline-4-carboxylicacid | 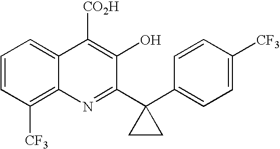 | 17 |
| 64 | 3-hydroxy-6,8-dimethyl-2-(1-phenylcyclopropyl)quinoline-4-carboxylic acid | 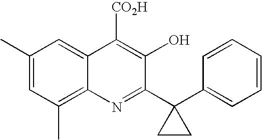 | 20 |
| 65 | 8-ethyl-2-(1-(4-fluorophenyl)cyclopropyl)-3-hydroxyquinoline-4-carboxylic acid | 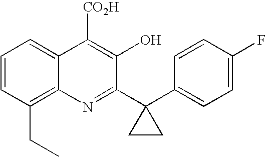 | 31 |
| 66 | 7-ethyl-2-(1-(4-fluorophenyl)cyclopropyl)-3-hydroxyquinoline-4-carboxylic acid | 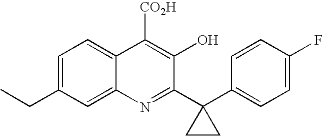 | ≦10 |
| 67 | 6-chloro-2-(1-(4-fluorophenyl)cyclopropyl)-3-hydroxyquinoline-4-carboxylic acid | 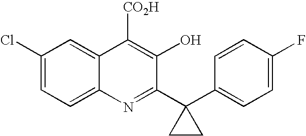 | ≦10 |
| 68 | 7-chloro-2-(1-(4-fluorophenyl)cyclopropyl)-3-hydroxyquinoline-4-carboxylic acid | 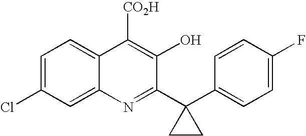 | ≦10 |
| 69 | 2-(1-(4-fluorophenyl)cyclopropyl)-3-hydroxy-6,8-dimethylquinoline-4-carboxylic acid | 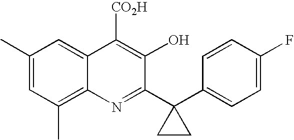 | 11 |
| 70 | 6-ethyl-2-(1-(4-fluorophenyl)cyclopropyl)-3-hydroxyquinoline-4-carboxylic acid | 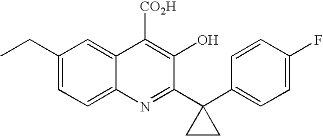 | 10 |
| 71 | 2-(1-(4-fluorophenyl)cyclopropyl)-3-hydroxy-8-(thiophen-3-yl)quinoline-4-carboxylic acid | 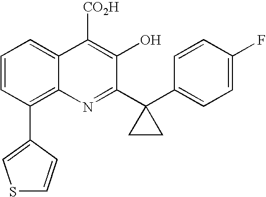 | 64 |
| 72 | 6-bromo-2-(1-(4-fluorophenyl)cyclopropyl)-3-hydroxyquinoline-4-carboxylic acid | 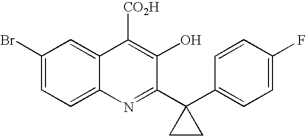 | 12 |
| 73 | 8-chloro-2-(1-(4-fluorophenyl)cyclopropyl)-3-hydroxyquinoline-4-carboxylic acid | 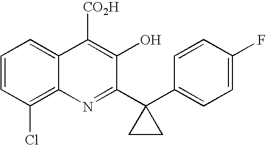 | ≦10 |
| 74 | 7-bromo-2-(1-(4-fluorophenyl)cyclopropyl)-3-hydroxyquinoline-4-carboxylic acid | 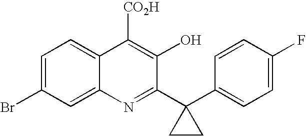 | 10 |
| 75 | 8-bromo-2-(1-(4-fluorophenyl)cyclopropyl)-3-hydroxyquinoline-4-carboxylic acid | 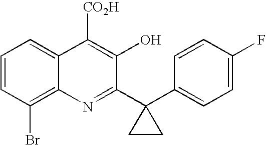 | ≦10 |
| 76 | 2-(1-(4-fluorophenyl)cyclopropyl)-8-(1,1,1,3,3,3-hexafluoro-2-hydroxypropan-2-yl)-3-hydroxyquinoline-4-carboxylic acid | 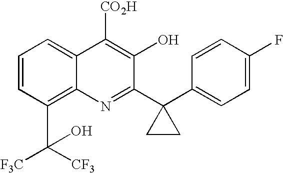 | 43 |
| 77 | 2-(1-(4-fluorophenyl)cyclopropyl)-3-hydroxy-8-phenylquinoline-4-carboxylic acid | 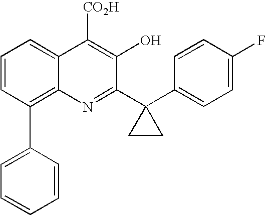 | 37 |
| 78 | 2-(1-(4-fluorophenyl)cyclopropyl)-3-hydroxy-8-methylquinoline-4-carboxylic acid | 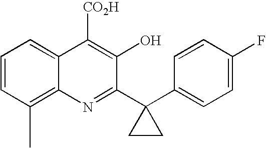 | 10 |
| 79 | 2-(1-(4-fluorophenyl)cyclopropyl)-3-hydroxy-6-methoxyquinoline-4-carboxylic acid | 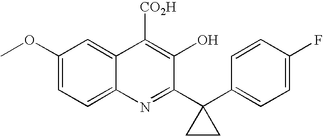 | ≦10 |
| 80 | 2-(1-(4-fluorophenyl)cyclopropyl)-3-hydroxy-7,8,9,10-tetrahydrobenzo[h]quinoline-4-carboxylic acid | 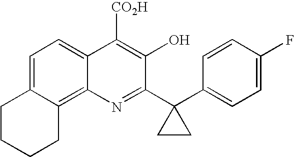 | 59 |
| 81 | 2-(1-(4-fluorophenyl)cyclopropyl)-3-hydroxy-7,8-dimethylquinoline-4-carboxylic acid | 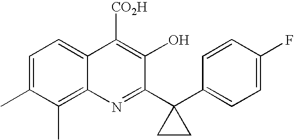 | 23 |
| 82 | 8-ethyl-2-(1-tolylcyclopropyl)-3-hydroxyquinoline-4-carboxylic acid | 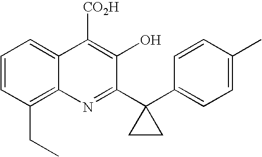 | 38 |
| 83 | 8-methyl-2-(1-p-tolylcyclopropyl)-3-hydroxyquinoline-4-carboxylic acid | 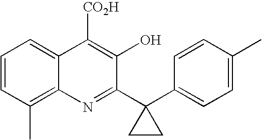 | 12 |
| 84 | 3-hydroxy-6,8-dimethyl-2-(1-p-tolylcyclopropyl)quinoline-4-carboxylic acid | 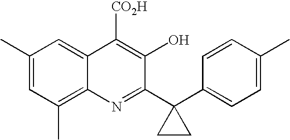 | 11 |
| 85 | 8-(1,1,1,3,3,3-hexafluoro-2-hydroxypropan-2-yl)-3-hydroxy-2-(1-p-tolylcyclopropyl)quinoline-4-carboxylic acid | 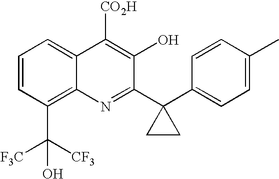 | 59 |
| 86 | 3-hydroxy-8-isopropyl-2-(1-p-tolylcyclopropyl)quinoline-4-carboxylic acid | 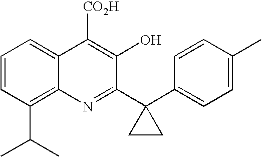 | 54 |
| 87 | 8-ethyl-3-hydroxy-2-(1-(4-(trifluoromethyl)phenyl)cyclopropyl)quinoline-4-carboxylicacid | 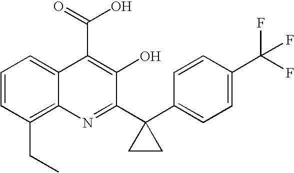 | 61 |
| 88 | 3-hydroxy-8-isopropyl-2-(1-(4-(trifluoromethyl)phenyl)cyclopropyl)quinoline-4-carboxylicacid | 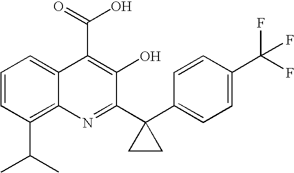 | 34 |
| 89 | 7-ethyl-3-hydroxy-2-(1-(4-(trifluoromethyl)phenyl)cyclopropyl)quinoline-4-carboxylicacid | 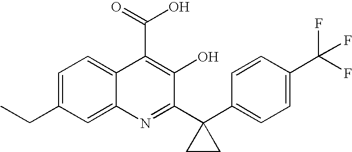 | 10 |
| 90 | 3-hydroxy-6-(trifluoromethoxy)-2-(1-(4-(trifluoromethyl)phenyl)cyclopropyl)quinoline-4-carboxylicacid | 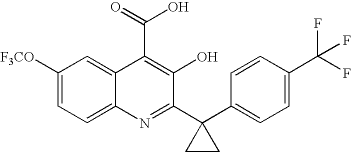 | 47 |
| 91 | 3-hydroxy-8-(thiophen-3-yl)-2-(1-(4-(trifluoromethyl)phenyl)cyclopropyl)quinoline-4-carboxylicacid | 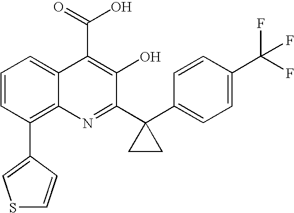 | ≦10 |
| 92 | 3-hydroxy-8-phenyl-2-(1-(4-(trifluoromethyl)phenyl)cyclopropyl)quinoline-4-carboxylicacid | 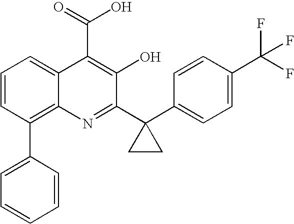 | ≦10 |
| 93 | 3-hydroxy-2-(1-(4-(trifluoromethyl)phenyl)cyclopropyl)-7,8,9,10-tetrahydrobenzo[h]quinoline-4-carboxylic acid | 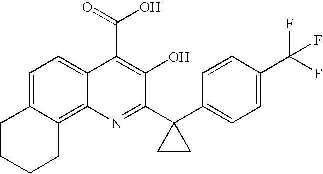 | 33 |
| 94 | 2-(1-(4-chlorophenyl)cyclopropyl)-3-hydroxy-6-methyl-8-(trifluoromethyl)quinoline-4-carboxylic acid | 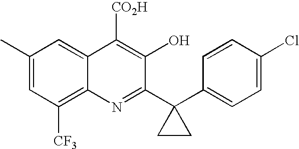 | 65 |
| 95 | 6-chloro-2-(1-(4-chlorophenyl)cyclopropyl)-3-hydroxy-8-(trifluoromethyl)quinoline-4-carboxylic acid | 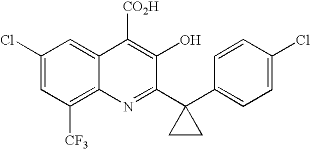 | ≦10 |
| 96 | 2-(1-(4-chlorophenyl)cyclopropyl)-3-hydroxy-6-phenyl-8-(trifluoromethyl)quinoline-4-carboxylic acid | 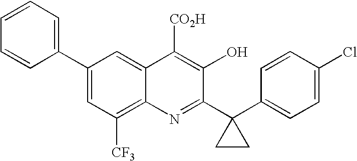 | ≦10 |
| 97 | 2-(1-(4-chlorophenyl)cyclopropyl)-3-hydroxy-8-methyl-6-(trifluoromethyl)quinoline-4-carboxylic acid | 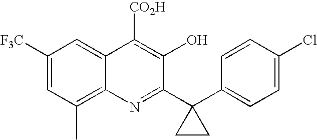 | 40 |
| 98 | 2-(1-(4-chlorophenyl)cyclopropyl-6-ethyl-3-hydroxy-8-(trifluoromethyl)quinoline-4-carboxylic acid | 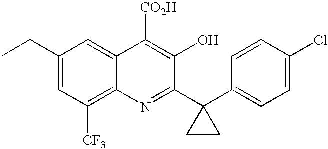 | ≦10 |
| 99 | 2-(1-(4-chlorophenyl)cyclopropyl)-8-ethyl-3-hydroxy-6-(trifluoromethyl)quinoline-4-carboxylic acid | 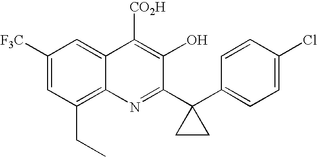 | ≦10 |
| 100 | 2-(1-(4-chlorophenyl)cyclopropyl)-3-hydroxy-8-phenyl-6-(trifluoromethyl)quinoline-4-carboxylic acid | 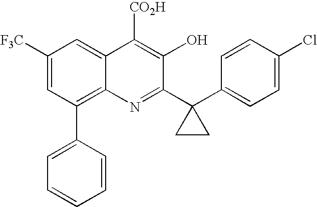 | ≦10 |
| 101 | 3-hydroxy-6-methyl-2-(1-phenylcyclopropyl)-8-(trifluoromethyl)quinoline-4-carboxylic acid | 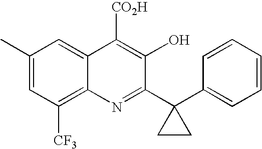 | 40 |
| 102 | 3-hydroxy-6-phenyl-2-(1-phenylcyclopropyl)-8-(trifluoromethyl)quinoline-4-carbpxylic acid | 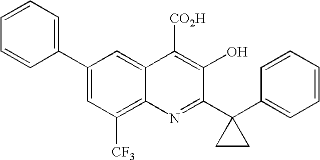 | ≦10 |
| 103 | 6-bromo-2-(1-(4-chlorophenyl)cyclopropyl)-3-hydroxy-8-(trifluoromethyl)quinoline-4-carboxylic acid | 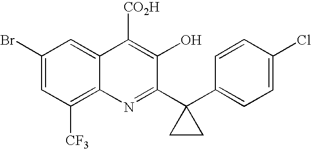 | ≦10 |
| 104 | 6-ethyl-3-hydroxy-2-(1-phenylcyclopropyl)-8-(trifluoromethyl)quinoline-4-carboxylic acid | 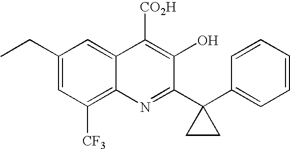 | 40 |
| 105 | 3-hydroxy-2-(1-(4-chlorophenyl)cyclopropyl)-6,8-bis(trifluoromethyl)quinoline-4-carboxylic acid | 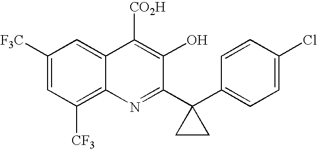 | ≦10 |
| 106 | 2-(1-(4-phenyl)cyclopropyl-3-hydroxy-6,8-bis-(trifluoromethyl)quinoline-4-carboxylic acid | 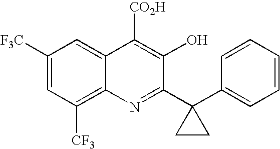 | 41 |
| 107 | 6-bromo-3-hydroxy-2-(1-phenylcyclopropyl)-8-(trifluoromethyl)quinoline-4-carboxylic acid | 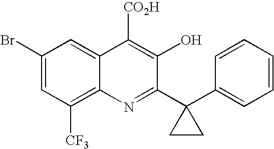 | 41 |
| 108 | 2-(1-(4-chlorophenyl)cycloproyl)-3-hydroxyquinoline-4,8-dicarboxylicacid | 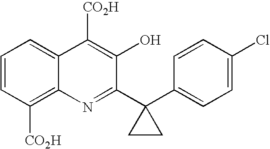 | 12 |
| 109 | 2-(1-(4-chloro-phenyl)-cyclopropyl)-8-cyclopropyl-3-hydroxy-quinoline-4-carboxylic acid | 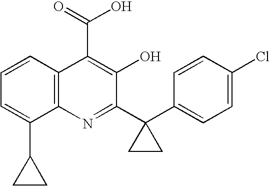 | 57 |
| 110 | 8-cyclopropyl-3-hydroxy-2-(1-phenyl-cyclopropyl)-quinoline-4-carboxylic acid | 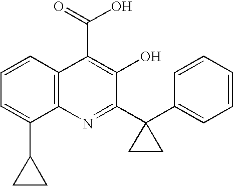 | 13 |
| 111 | 3-hydroxy-2-(1-phenyl-cyclopropylmethyl)-8-trifluoromethyl-quinoline-4-carboxylic acid | 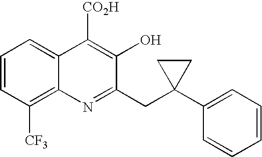 | 46 |
| 112 | 2-(1-benzyl-cyclopropyl)-3-hydroxy-8-trifluoromethyl-quinoline-4-carboxylic acid | 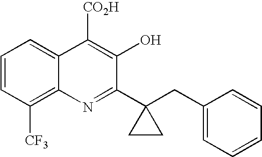 | 43 |
| 113 | 3-hydroxy-7,8-dimethyl-2-(1-p-tolyl-cyclopropyl)-quinoline-4-carboxylicacid | 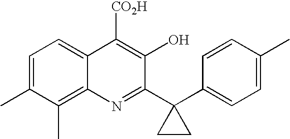 | 24 |
| 114 | 3-hydroxy-2-(2-phenylpropan-2-yl)-7,8,9,10-tetrahydrobenzo[h]quinoline-4-carboxylic acid | 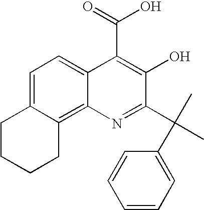 | 69 |
| 115 | 3-hydroxy-7,8-dimethyl-2-(2-phenylpropan-2-yl)quinoline-4-carboxylic acid | 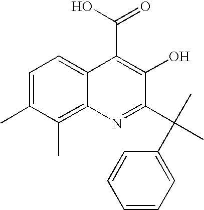 | 60 |
| 116 | 3-hydroxy-8-isopropyl-2-(2-phenylpropan-2-yl)quinoline-4-carboxylic acid | 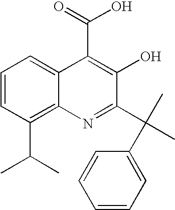 | 68 |
| 117 | 3-hydroxy-2-(2-phenylpropan-2-yl)-8-(trifluoromethyl)quinoline-4-carboxylic acid | 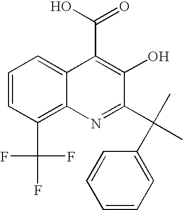 | 55 |
| 118 | 2-(2-(4-chlorophenyl)propan-2-yl)-3-hydroxy-8-isopropylquinoline-4-carboxylic acid | 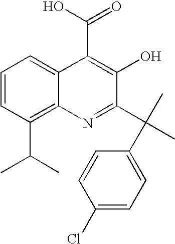 | ≦10 |
| 119 | 2-(2-(4-chlorophenyl)propan-2-yl)-3-hydroxy-8-(trifluoromethyl)quinoline-4-carboxylic acid | 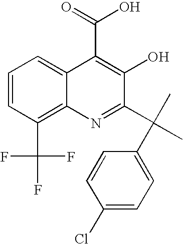 | ≦10 |
| 120 | 2-(2-(4-chlorophenyl)propan-2-yl)-3-hydroxy-7,8,9,10-tetrahydrobenzo[h]quinoline-4-carboxylic acid | 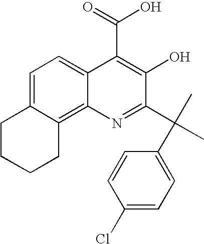 | ≦10 |
| 121 | 2-(2-(4-chlorophenyl)propan-2-yl)-3-hydroxy-7,8-dimethylquinoline-4-carboxylic acid | 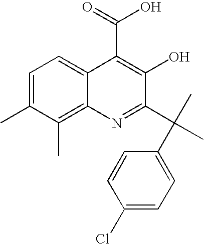 | 56 |
| 122 | 2-(2-(4-chlorophenyl)propan-2-yl)-8-(1,1,1,3,3,3-hexafluoro-2-hydroxypropan-2-yl)-3-hydroxyquinoline-4-carboxylic acid | 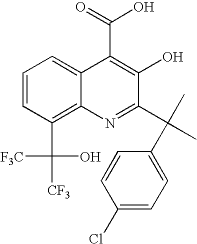 | ≦10 |
| 123 | 3-hydroxy-2-(1-phenylethyl)-8-(trifluoromethyl)quinoline-4-carboxylic acid | 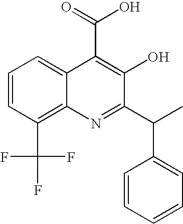 | 49 |
| 124 | 2-[1-(4-chlorophenyl)ethyl]-3-hydroxy-7,8,9,10-tetrahydrobenzo[h]quinoline-4-carboxylic acid | 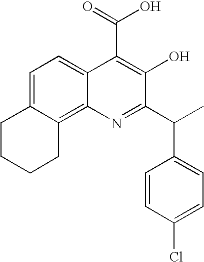 | ≦10 |
| 125 | 3-hydroxy-2-(1-phenylethyl)-7,8,9,10-tetrahydrobenzo[h]quinoline-4-carboxylic acid | 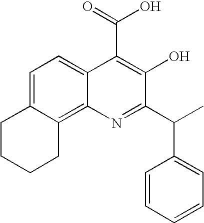 | 90 |
| 126 | 3-hydroxy-2-(1-phenylpropyl)-8-(trifluoromethyl)quinoline-4-carboxylic acid | 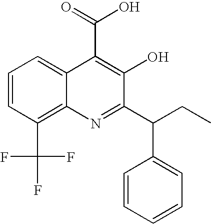 | 61 |
| 127 | 3-hydroxy-8-isopropyl-2-(1-phenylpropyl)quinoline-4-carboxylicacid | 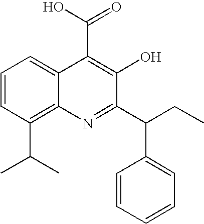 | 65 |
| 128 | 3-hydroxy-7,8-dimethyl-2-(1-phenylpropyl)quinoline-4-carboxylicacid | 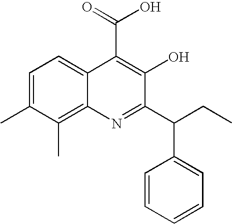 | 64 |
| 129 | 3-hydroxy-2-(2-methyl-1-phenylpropyl)-8-(trifluoromethyl)quinoline-4-carboxylic acid | 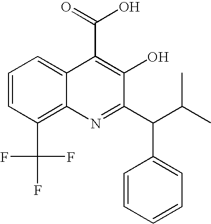 | 60 |
| 130 | 3-hydroxy-8-isopropyl-2-(2-methyl-1-phenylpropyl)quinoline-4-carboxylic acid | 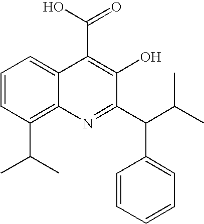 | 40 |
| 131 | 3-hydroxy-7,8-dimethyl-2-(2-methyl-1-phenylpropyl)quinoline-4-carboxylic acid | 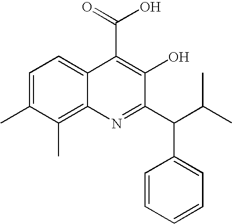 | 90 |
| 132 | 3-hydroxy-2-(1-phenylpropan-2-yl)-8-(trifluoromethyl)quinoline-4-carboxylic acid | 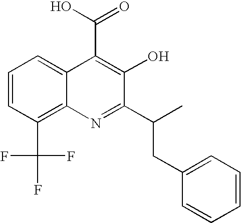 | 49 |
| 133 | 3-hydroxy-8-isopropyl-2-(1-phenylpropan-2-yl)quinoline-4-carboxylic acid | 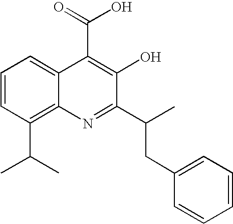 | 64 |
| 134 | 3-hydroxy-7,8-dimethyl-2-(1-phenylpropan-2-yl)quinoline-4-carboxylic acid | 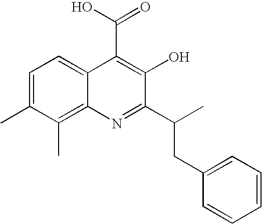 | 49 |
| 135 | 3-hydroxy-2-(2-phenylpropyl)-8-(trifluoromethyl)quinoline-4-carboxylic acid | 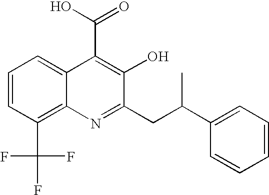 | 46 |
| 136 | 3-hydroxy-8-isopropyl-2-(2-phenylpropyl)quinoline-4-carboxylicacid | 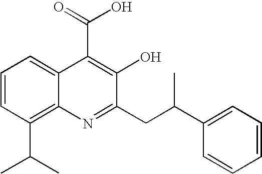 | 64 |
| 137 | 3-hydroxy-7,8-dimethyl-2-(2-phenylpropyl)quinoline-4-carboxylicacid | 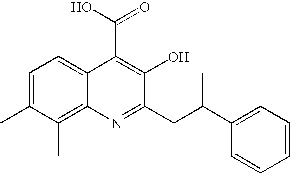 | 31 |
| 138 | 2-(4-chlorobenzyl)-3-[(morpholin-4-ylcarbonyl)oxy]-7,8,9,10-tetrahydrobenzo[h]quinoline-4-carboxylic acid | 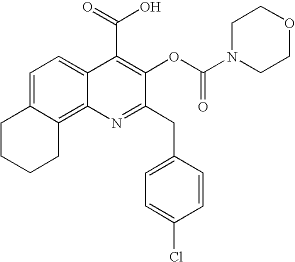 | 16 |

As those skilled in the art will appreciate, numerous changes and modifications can be made to the above-described embodiments of the present teachings without departing from the spirit of the present teachings. It is intended that all such variations fall within the scope of the present teachings.

What is claimed is:

1. A compound of formula I: 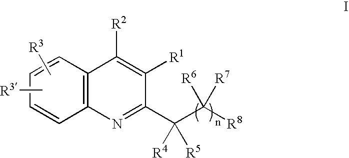
or a pharmaceutically acceptable salt, hydrate, or ester thereof, wherein:
R¹ is —OR⁹, —C(O)R¹⁰, —C(O)OR⁹, —C(O)NR¹⁰R¹¹, —C(S)R¹⁰, —C(S)OR⁹, —C(S)NR¹⁰R¹¹, —C(NR¹⁰)R¹⁰, —C(NR¹⁰)NR¹⁰R¹¹, —NR¹⁰R¹¹, —NR¹¹C(O)R¹⁰, —NR¹¹C(O)NR¹⁰R¹¹, —NR¹¹C(NR¹⁰)NR¹⁰R¹¹, NR¹¹S(O)ₘR¹⁰, or —NR¹¹S(O)ₘNR¹⁰R¹¹;
R² is —C(O)OR⁹, —C(O)NR¹⁰R¹¹, or a carboxylic acid bioisostere;
R³ and R^{3′} independently are H, —CN, —NO₂, halogen, —OR⁹, —NR¹⁰R¹¹, —S(O)ₘR¹⁰, —S(O)ₘOR⁹, —S(O)ₘNR¹⁰R¹¹, —C(O)R¹⁰, —C(O)OR⁹, —C(O)NR¹⁰R¹¹, —C(S)R¹⁰, —C(S)OR⁹, —C(S)NR¹⁰R¹¹, —C(NR¹⁰)NR¹⁰R¹¹, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups; or
R³ and R^{3′}, together with the carbon atoms to which each is attached, form a C₄₋₁₄ cycloalkyl group, a C₆₋₁₄ aryl group, a 4-14 membered cycloheteroalkyl group, or a 5-14 membered heteroaryl group, wherein each of the C₄₋₁₄ cycloalkyl group, the C₆₋₁₄ aryl group, the 4-14 membered cycloheteroalkyl group, and the 5-14 membered heteroaryl group optionally is substituted with 1-4-Z-R¹² groups;
R⁴ and R⁵ independently are H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups; or
R⁴ and R⁵, together with their respective common carbon atom, form a C₃₋₁₄ cycloalkyl group, a C₆₋₁₄ aryl group, a 3-14 membered cycloheteroalkyl group, or a 5-14 membered heteroaryl group, wherein each of the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;
R⁶ and R⁷, at each occurrence, independently are H, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups; or
R⁶ and R⁷, together with their respective common carbon atom, form a C₃₋₁₄ cycloalkyl group, a C₆₋₁₄ aryl group, a 3-14 membered cycloheteroalkyl group, or a 5-14 membered heteroaryl group, wherein each of the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;
provided that at least one of R⁴ and R⁵ and R⁶ and R⁷, together with their respective common carbon atom, form a C₃₋₁₄ cycloalkyl group, a C₆₋₁₄ aryl group, a 3-14 membered cycloheteroalkyl group, or a 5-14 membered heteroaryl group, wherein each of the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;
R⁸ is a C₆₋₁₄ aryl group or a 5-14 membered heteroaryl group, wherein each of the C₆₋₁₄ aryl group and the 5-14 membered heteroaryl group optionally is substituted with 1-4-Z-R¹² groups;
R⁹, at each occurrence, independently is H, —C(O)R¹⁰, —C(O)NR¹⁰R¹¹, —C(S)R¹⁰, —C(S)NR¹⁰R¹¹, —C(NR¹⁰)R¹⁰, —C(NR¹⁰)NR¹⁰R¹¹, —S(O)ₘR¹⁰, i-S(O)ₘNR¹⁰R¹¹, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;
R¹⁰ and R¹¹, at each occurrence, independently are H, —OH, —SH, —S(O)₂OH, —C(O)OH, —C(O)NH₂, —C(S)NH₂, —OC₁₋₁₀ alkyl, —C(O)—C₁₋₁₀ alkyl, —C(O)—OC₁₋₁₀ alkyl, —OC₆₋₁₄ aryl, —C(O)—C₆₋₁₄ aryl, —C(O)—OC₆₋₁₄ aryl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —C(O)NH—C₆₋₁₄ aryl, —S(O)ₘ—C₁₋₁₀ alkyl, t-S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹² groups;
R¹², at each occurrence, independently is halogen, —CN, —NO₂, oxo, —O-Z-R¹³, —NR¹³-Z-R¹⁴, —N(O)R¹³-Z-R¹⁴, S(O)ₘR¹³, —S(O)ₘO-Z-R¹³, S(O)ₘNR¹³-Z-R¹⁴, —C(O)R¹³, —C(O)O-Z-R¹³, —C(O)NR¹³-Z-R¹⁴, n-C(S)NR¹³-Z-R¹⁴, —Si(C₁₋₁₀ alkyl)₃, a C₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹⁵ groups;
R¹³ and R¹⁴, at each occurrence, independently are H, —OH, —SH, —S(O)₂OH, —C(O)OH, —C(O)NH₂, —C(S)NH₂, —OC₁₋₁₀ alkyl, —C(O)—C₁₋₁₀ alkyl, —C(O)—OC₁₋₁₀ alkyl, —C(S)N(C₁₋₁₀ alkyl)₂, —C(S)NH—C₁₋₁₀ alkyl, —C(O)NH—C₁₋₁₀ alkyl, —C(O)N(C₁₋₁₀ alkyl)₂, —S(O)ₘ—C₁₋₁₀ alkyl, —S(O)ₘ—OC₁₋₁₀ alkyl, a C₁₋₁₀ alkyl group, a C₂₋₁₀ alkenyl group, a C₂₋₁₀ alkynyl group, a C₃₋₁₄ cycloalkyl group, a C₆₋₁₄ aryl group, a 3-14 membered cycloheteroalkyl group, or a 5-14 membered heteroaryl group, wherein each of the C₁₋₁₀ alkyl group, the C₂₋₁₀ alkenyl group, the C₂₋₁₀ alkynyl group, the C₃₋₁₄ cycloalkyl group, the C₆₋₁₄ aryl group, the 3-14 membered cycloheteroalkyl group, and the 5-14 membered heteroaryl group optionally is substituted with 1-4-Z-R¹⁵ groups;
R¹⁵, at each occurrence, independently is halogen, —CN, —NO₂, oxo, —OH, f) —NH₂, —NH(C₁₋₁₀ alkyl), —N(C₁₋₁₀ alkyl)₂, —S(O)ₘH, —S(O)ₘ—C₁₋₁₀ alkyl, —S(O)₂OH, —S(O)ₘ—OC₁₋₁₀ alkyl, —CHO, —C(O)—C₁₋₁₀ alkyl, —C(O)OH, —C(O)—OC₁₋₁₀ alkyl, —C(O)NH₂, —C(O)NH—C₁₋₁₀ alkyl, —C(O)N(C₁₋₁₀ alkyl)₂, —C(S)NH₂, —C(S)NH—C₁₋₁₀ alkyl, —C(S)N(C₁₋₁₀ alkyl)₂, —S(O)ₘNH₂, —S(O)ₘNH(C₁₀ alkyl), —S(O)ₘN(C₁₋₁₀ alkyl)₂, —Si(C₁₋₁₀ alkyl)₃, a C₁₋₁₀ alkyl group, a C₂₋₁₀ alkenyl group, a C₂₋₁₀ alkynyl group, a C₁₋₁₀ alkoxy group, a C₁₋₁₀ haloalkyl group, a C₃₋₁₄ cycloalkyl group, a C₆₋₁₄ aryl group, a 3-14 membered cycloheteroalkyl group, or a 5-14 membered heteroaryl group;
Z, at each occurrence, independently is a divalent C₁₋₁₀ alkyl group, a divalent C₂₋₁₀ alkenyl group, a divalent C₂₋₁₀ alkynyl group, a divalent C₁₋₁₀ haloalkyl group, or a covalent bond;
m, at each occurrence, independently is 0, 1, or 2; and
n is 0, 1, or 2.

2. The compound of claim 1 or a pharmaceutically acceptable salt, hydrate, or ester thereof, wherein R¹ is —OR⁹, —OC(O)R¹⁰, or —NR¹⁰R¹¹; wherein R⁹, R¹⁰, and R¹¹ are as defined in claim 1.

3. The compound of claim 2 or a pharmaceutically acceptable salt, hydrate, or ester thereof, wherein R¹ is —OH.

4. The compound of claim 1 or a pharmaceutically acceptable salt, hydrate, or ester thereof, wherein R² is —C(O)OH.

5. The compound of claim 1 or a pharmaceutically acceptable salt, hydrate, or ester thereof, wherein the compound has formula Ia, formula Ib, formula Ic, formula Id, formula Ie, or formula If: 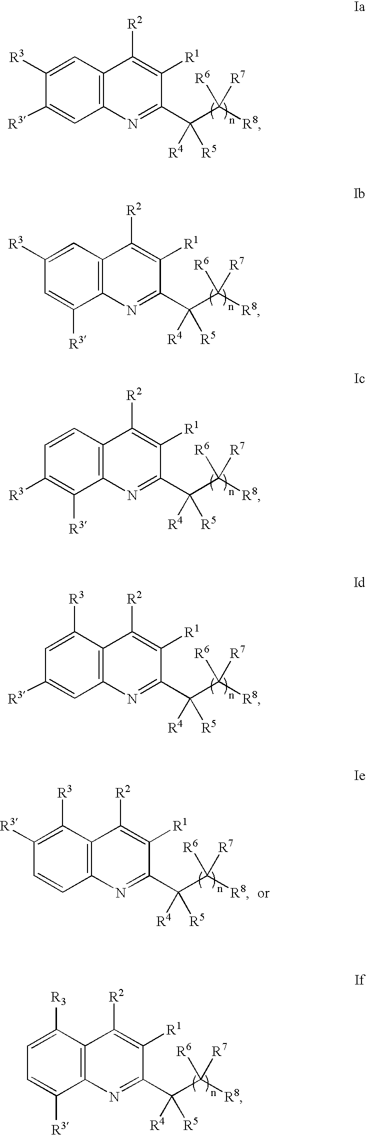
wherein R¹, R², R³, R^{3′}, R⁴, R⁵, R⁶, R⁷, R⁸, and n are as defined in claim 1.

6. The compound of claim 1 or a pharmaceutically acceptable salt, hydrate, or ester thereof, wherein R³ and R^{3′} independently are H, halogen, —OR⁹, —C(O)OR⁹, a C₁₋₁₀ alkyl group, a C₃₋₁₄ cycloalkyl group, a C₆₋₁₄ aryl group, or a 5-14 membered heteroaryl group, wherein each of the C₁₋₁₀ alkyl group, the C₃₋₁₄ cycloalkyl group, the C₆₋₁₄ aryl group, and the 5-14 membered heteroaryl group optionally is substituted with 1-4-Z-R¹² groups; and R⁹, R¹², and Z are as defined in claim 1.

7. The compound of claim 1 or a pharmaceutically acceptable salt, hydrate, or ester thereof, wherein R³ and R^{3′} independently are H, halogen, —CF₃, a C₁₋₁₀ alkyl group, a C₃₋₁₄ cycloalkyl group, —CO₂H, —OC₁₋₁₀ alkyl, —OCF₃, —C(CF₃)₂OH, phenyl, or a 5-14 membered heteroaryl group.

8. The compound of claim 1 or a pharmaceutically acceptable salt, hydrate, or ester thereof, wherein one of R³ and R^{3′} is H and the other is —CF₃.

9. The compound of claim 1 or a pharmaceutically acceptable salt, hydrate, or ester thereof, wherein one of R³ and R^{3′} is —C(CF₃)₂OH.

10. The compound of claim 1 or a pharmaceutically acceptable salt, hydrate, or ester thereof, wherein R³ and R^{3′}, together with the carbon atoms to which each is attached, form a C₄₋₁₄ cycloalkyl group or a 4-14 membered cycloheteroalkyl group, wherein each of the C₄₋₁₄ cycloalkyl group and the 4-14 membered cycloheteroalkyl group optionally is substituted with 1-4-Z-R¹² groups, and Z and R¹² are as defined in claim 1.

11. The compound of claim 10 or a pharmaceutically acceptable salt, hydrate, or ester thereof, wherein R³ and R^{3′}, together with the carbon atoms to which each is attached, form a C₆ cycloalkyl group.

12. The compound of claim 11, or a pharmaceutically acceptable salt, hydrate, or ester thereof, wherein the compound has formula Ig: 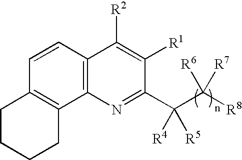
wherein R¹, R², R⁴, R⁵, R⁶, R⁷, R⁸ and n are as defined in claim 1

13. The compound of claim 1 or a pharmaceutically acceptable salt, hydrate, or ester thereof, wherein at least one of R⁴ and R⁵, and R⁶ and R⁷, together with their respective common carbon atom, form a C₃₋₁₄ cycloalkyl group optionally is substituted with 1-4-Z-R¹² groups, and Z and R¹² are as defined herein.

14. The compound of claim 13 or a pharmaceutically acceptable salt, hydrate, or ester thereof, wherein R⁴ and R⁵, together with their common carbon atom, form a C₃₋₁₄ cycloalkyl group optionally is substituted with 1-4-Z-R¹² groups, and Z and R¹² are as defined in claim 1.

15. The compound of claim 14 or a pharmaceutically acceptable salt, hydrate, or ester thereof, wherein R⁴ and R⁵, together with their common carbon atom, form a cyclopropyl group or a cyclobutyl group.

16. The compound of claim 1 or a pharmaceutically acceptable salt, hydrate, or ester thereof, wherein n is 0.

17. The compound of claim 1 or a pharmaceutically acceptable salt, hydrate, or ester thereof, wherein the compound has formula II: 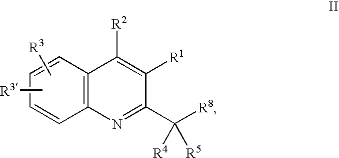
wherein R⁴ and R⁵, together with their common carbon atom, form a C₃₋₁₄ cycloalkyl group optionally substituted with 1-4-Z-R¹² groups, and R¹, R², R³, R^{3′}, R⁸, R¹², and Z are as defined in claim 1.

18. The compound of claim 17, or a pharmaceutically acceptable salt, hydrate, or ester thereof, wherein the compound has formula IIg: 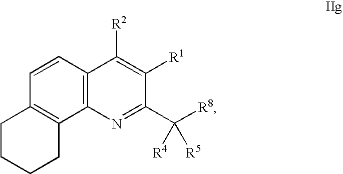
wherein R¹, R², R⁴, R⁵, and R⁸ are as defined in claim 1.

18. The compound of claim 1 or a pharmaceutically acceptable salt, hydrate, or ester thereof, wherein n is 1.

19. The compound of claim 1 or a pharmaceutically acceptable salt, hydrate, or ester thereof, wherein the compound has formula IVa or formula IVb: 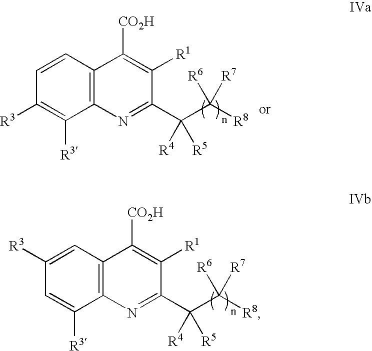
wherein R¹, R³, R^{3′}, R⁴, R⁵, R⁶, R⁷, R⁸, and n are as defined in claim 1.

21. The compound of claim 1 or a pharmaceutically acceptable salt, hydrate, or ester thereof, wherein n is 1 and R⁶ and R⁷ independently are H or a C₁₋₆ alkyl group, wherein the C₁₋₆ alkyl group optionally is substituted with 1-4-Z-R¹² groups and Z and R¹² are as defined in claim 1.

22. The compound of claim 1 or a pharmaceutically acceptable salt, hydrate, or ester thereof, wherein n is 1 and R⁶ and R⁷, together with their respective common carbon atom, form a C₃₋₁₄ cycloalkyl group optionally substituted with 1-4-Z-R¹² groups, and Z and R¹² are as defined in claim 1.

23. The compound of claim 22 or a pharmaceutically acceptable salt, hydrate, or ester thereof, wherein R⁶ and R⁷, together with their respective common carbon atom, form a cyclopropyl group or a cyclobutyl group.

24. The compound of claim 22 or a pharmaceutically acceptable salt, hydrate, or ester thereof, wherein R⁴ and R⁵ independently are H or a C₁₋₆ alkyl group optionally substituted with 1-4-Z-R¹² groups, and Z and R¹² are as defined in claim 1.

25. The compound of claim 1 or a pharmaceutically acceptable salt, hydrate, or ester thereof, wherein R⁸ is a C₆₋₁₄ aryl group substituted with a halogen, —O-Z-R³, a C₁₋₁₀ alkyl group, or a C₁₋₁₀ haloalkyl group, wherein Z and R¹³ are as defined in claim 1.

26. The compound of claim 25 or a pharmaceutically acceptable salt, hydrate, or ester thereof, wherein R⁸ is a phenyl group substituted with a halogen, —O-Z-R¹³, a C₁₋₁₀ alkyl group, or a C₁₋₁₀ haloalkyl group, wherein Z and R¹³ are as defined in claim 1.

27. A compound of claim 1 wherein the compound is selected from 2-(1-(4-chlorophenyl)cyclopropyl)-3-hydroxy-8-(trifluoromethoxy)quinoline-4-carboxylic acid; 2-(1-(4-chlorophenyl)cyclopropyl)-8-ethyl-3-hydroxyquinoline-4-carboxylic acid; 8-sec-butyl-2-(1-(4-chlorophenyl)cyclopropyl)-3-hydroxyquinoline-4-carboxylic acid; 8-tert-butyl-2-(1-(4-chlorophenyl)cyclopropyl)-3-hydroxyquinoline-4-carboxylic acid; 8-chloro-2-(1-(4-chlorophenyl)cyclopropyl)-3-hydroxyquinoline-4-carboxylic acid; 2-(1-(4-chlorophenyl)cyclopropyl)-3-hydroxy-8-phenylquinoline-4-carboxylic acid; 2-(1-(4-chlorophenyl)cyclopropyl)-8-fluoro-3-hydroxyquinoline-4-carboxylic acid; 2-(1-(4-chlorophenyl)cyclopropyl)-3-hydroxyquinoline-4-carboxylic acid; 2-(1-(4-chlorophenyl)cyclopropyl)-3-hydroxyquinoline-4-carboxylic acid; 8-bromo-2-(1-(4-chlorophenyl)cyclopropyl)-3-hydroxyquinoline-4-carboxylic acid; and 2-(1-(4-chlorophenyl)cyclopropyl)-3-hydroxy-6,8-dimethylquinoline-4-carboxylic acid, or a pharmaceutically acceptable salt, hydrate, or ester thereof.

28. A compound of claim 1 wherein the compound is selected from 2-(1-(4-chlorophenyl)cyclopropyl)-3-hydroxy-8-methylquinoline-4-carboxylic acid; 2-(1-(4-chlorophenyl)cyclopropyl)-7-ethyl-3-hydroxyquinoline-4-carboxylic acid; 2-(1-(4-chlorophenyl)cyclopropyl)-3-hydroxy-7-methylquinoline-4-carboxylic acid; 8-ethyl-3-hydroxy-2-(1-phenylcyclopropyl)quinoline-4-carboxylic acid; 8-sec-butyl-3-hydroxy-2-(1-phenylcyclopropyl)quinoline-4-carboxylic acid; 7-chloro-2-(1-(4-chlorophenyl)cyclopropyl)-3-hydroxyquinoline-4-carboxylic acid; 2-(1-(4-chlorophenyl)cyclopropyl)-6-fluoro-3-hydroxyquinoline-4-carboxylic acid; 6-bromo-2-(1-(4-chlorophenyl)cyclopropyl)-3-hydroxyquinoline-4-carboxylic acid; 2-(1-(4-chlorophenyl)cyclopropyl)-3-hydroxy-6-methylquinoline-4-carboxylic acid; and 2-(1-(4-chlorophenyl)cyclopropyl)-3-hydroxy-6,8-dimethylquinoline-4-carboxylic acid, or a pharmaceutically acceptable salt, hydrate, or ester thereof.

29. A compound of claim 1 wherein the compound is selected from 2-(1-(4-chlorophenyl)cyclopropyl)-3-hydroxy-6-(trifluoromethoxy)quinoline-4-carboxylic acid; 6-chloro-2-(1-(4-chlorophenyl)cyclopropyl)-3-hydroxyquinoline-4-carboxylic acid; 2-(1-(4-chlorophenyl)cyclopropyl)-3,6-dihydroxyquinoline-4-carboxylic acid; 2-(1-(4-chlorophenyl)cyclopropyl)-3-hydroxy-6-(trifluoromethyl)quinoline-4-carboxylic acid; 2-(1-(4-chlorophenyl)cyclopropyl)-3-hydroxy-6-isopropylquinoline-4-carboxylic acid; 7-chloro-3-hydroxy-2-(1-phenylcyclopropyl)quinoline-4-carboxylic acid; 6-ethyl-3-hydroxy-2-(1-phenylcyclopropyl)quinoline-4-carboxylic acid; 2-(1-(4-chlorophenyl)cyclopropyl)-3,6-dihydroxyquinoline-4-carboxylic acid; and 6-chloro-3-hydroxy-2-(1-phenylcyclopropyl)quinoline-4-carboxylic acid, or a pharmaceutically acceptable salt, hydrate, or ester thereof.

30. A compound of claim 1 wherein the compound is selected from 3-hydroxy-8-methyl-2-(1-phenylcyclopropyl)quinoline-4-carboxylic acid; 3-hydroxy-2-(1-phenylcyclopropyl)-6-(trifluoromethyl)quinoline-4-carboxylic acid; 3-hydroxy-6-methyl-2-(1-phenylcyclopropyl)quinoline-4-carboxylic acid; 3-hydroxy-2-(1-phenylcyclopropyl)-8-(trifluoromethyl)quinoline-4-carboxylic acid; 3-hydroxy-2-(1-phenylcyclopropyl)-8-(thiophen-3-yl)quinoline-4-carboxylic acid; 2-(1-(4-chlorophenyl)cyclopropyl)-3-hydroxy-7,8,9,10-tetrahydrobenzo[h]quinoline-4-carboxylic acid; 2-(1-(4-chlorophenyl)cyclopropyl)-3-hydroxy-8-(thiophen-3-yl)quinoline-4-carboxylic acid; 2-(1-(4-chlorophenyl)cyclopropyl)-3-hydroxy-8-(trifluoromethyl)quinoline-4-carboxylic acid; 2-(1-(4-chlorophenyl)cyclopropyl)-3-hydroxy-8-isopropylquinoline-4-carboxylic acid; and 2-(1-(4-chlorophenyl)cyclopropyl)-3-hydroxy-7,8-dimethylquinoline-4-carboxylic acid, or a pharmaceutically acceptable salt, hydrate, or ester thereof.

31. A compound of claim 1 wherein the compound is selected from 2-(1-(4-chlorophenyl)cyclopropyl)-8-(1,1,1,3,3,3-hexafluoro-2-hydroxypropan-2-yl)-3-hydroxyquinoline-4-carboxylic acid; 3-hydroxy-2-(1-phenylcyclopropyl)-7,8,9,10-tetrahydrobenzo[h]quinoline-4-carboxylic acid; 3-hydroxy-7,8-dimethyl-2-(1-phenylcyclopropyl)quinoline-4-carboxylic acid; 3-hydroxy-8-isopropyl-2-(1-phenylcyclopropyl)quinoline-4-carboxylic acid; 3-hydroxy-8-phenyl-2-(1-phenylcyclopropyl)quinoline-4-carboxylic acid; 3-hydroxy-2-(1-phenylcyclopropyl)-8-(trifluoromethoxy)quinoline-4-carboxylic acid; 8-chloro-3-hydroxy-2-(1-phenylcyclopropyl)quinoline-4-carboxylic acid; 6-(1,1,1,3,3,3-hexafluoro-2-hydroxypropan-2-yl)-3-hydroxy-2-(1-phenylcyclopropyl)quinoline-4-carboxylic acid; 8-(1,1,1,3,3,3-hexafluoro-2-hydroxypropan-2-yl)-3-hydroxy-2-(1-phenylcyclopropyl)quinoline-4-carboxylic acid; and 3-hydroxy-2-(1-(4-methoxyphenyl)cyclopropyl)-8-(trifluoromethyl)quinoline-4-carboxylic acid, or a pharmaceutically acceptable salt, hydrate, or ester thereof.

32. A compound of claim 1 wherein the compound is selected from 3-hydroxy-2-(1-(4-methoxyphenyl)cyclopropyl)-7,8,9,10-tetrahydrobenzo[h]quinoline-4-carboxylic acid; 3-hydroxy-8-(trifluoromethyl)-2-(1-(4-(trifluoromethyl)phenyl)cyclopropyl)quinoline-4-carboxylic acid; 2-(1-(4-bromophenyl)cyclopropyl)-3-hydroxy-8-(trifluoromethyl)quinoline-4-carboxylic acid; 2-(1-(3-chlorophenyl)cyclopropyl)-3-hydroxy-8-(trifluoromethyl)quinoline-4-carboxylic acid; 2-(1-(2-chlorophenyl)cyclopropyl)-3-hydroxy-8-(trifluoromethyl)quinoline-4-carboxylic acid; 3-hydroxy-2-(1-(4-(trifluoromethoxy)phenyl)cyclopropyl)-8-(trifluoromethyl)quinoline-4-carboxylic acid; 3-hydroxy-8-(trifluoromethyl)-2-(1-(3-(trifluoromethyl)phenyl)cyclopropyl)quinoline-4-carboxylic acid; 2-(1-(4-chlorophenyl)cyclobutyl)-3-hydroxy-8-(trifluoromethyl)quinoline-4-carboxylic acid; 3-hydroxy-2-(1-(thiophen-3-yl)cyclopropyl)-8-(trifluoromethyl)quinoline-4-carboxylic acid; and 3-hydroxy-2-(1-(thiophen-2-yl)cyclopropyl)-8-(trifluoromethyl)quinoline-4-carboxylic acid, or a pharmaceutically acceptable salt, hydrate, or ester thereof.

33. A compound of claim 1 wherein the compound is selected from 2-(1-(4-fluorophenyl)cyclopropyl)-3-hydroxy-8-(trifluoromethyl)quinoline-4-carboxylic acid; 2-(1-(4-fluorophenyl)cyclopropyl)-3-hydroxy-8-isopropylquinoline-4-carboxylic acid; 3-hydroxy-8-(trifluoromethyl)-2-(1-(2-(trifluoromethyl)phenyl)cyclopropyl)quinoline-4-carboxylic acid; 3-hydroxy-6,8-dimethyl-2-(1-phenylcyclopropyl)quinoline-4-carboxylic acid; 8-ethyl-2-(1-(4-fluorophenyl)cyclopropyl)-3-hydroxyquinoline-4-carboxylic acid; 7-ethyl-2-(1-(4-fluorophenyl)cyclopropyl)-3-hydroxyquinoline-4-carboxylic acid; 6-chloro-2-(1-(4-fluorophenyl)cyclopropyl)-3-hydroxyquinoline-4-carboxylic acid; 7-chloro-2-(1-(4-fluorophenyl)cyclopropyl)-3-hydroxyquinoline-4-carboxylic acid; 2-(1-(4-fluorophenyl)cyclopropyl)-3-hydroxy-6,8-dimethylquinoline-4-carboxylic acid; and 6-ethyl-2-(1-(4-fluorophenyl)cyclopropyl)-3-hydroxyquinoline-4-carboxylic acid, or a pharmaceutically acceptable salt, hydrate, or ester thereof.

34. A compound of claim 1 wherein the compound is selected from 2-(1-(4-fluorophenyl)cyclopropyl)-3-hydroxy-8-(thiophen-3-yl)quinoline-4-carboxylic acid; 6-bromo-2-(1-(4-fluorophenyl)cyclopropyl)-3-hydroxyquinoline-4-carboxylic acid; 8-chloro-2-(1-(4-fluorophenyl)cyclopropyl)-3-hydroxyquinoline-4-carboxylic acid; 7-bromo-2-(1-(4-fluorophenyl)cyclopropyl)-3-hydroxyquinoline-4-carboxylic acid; 8-bromo-2-(1-(4-fluorophenyl)cyclopropyl)-3-hydroxyquinoline-4-carboxylic acid; 2-(1-(4-fluorophenyl)cyclopropyl)-8-(1,1,1,3,3,3-hexafluoro-2-hydroxypropan-2-yl)-3-hydroxyquinoline-4-carboxylic acid; 2-(1-(4-fluorophenyl)cyclopropyl)-3-hydroxy-8-phenylquinoline-4-carboxylic acid; 2-(1-(4-fluorophenyl)cyclopropyl)-3-hydroxy-8-methylquinoline-4-carboxylic acid; 2-(1-(4-fluorophenyl)cyclopropyl)-3-hydroxy-6-methoxyquinoline-4-carboxylic acid; and 2-(1-(4-fluorophenyl)cyclopropyl)-3-hydroxy-7,8,9,10-tetrahydrobenzo[h]quinoline-4-carboxylic acid, or a pharmaceutically acceptable salt, hydrate, or ester thereof.

35. A compound of claim 1 wherein the compound is selected from 2-(1-(4-fluorophenyl)cyclopropyl)-3-hydroxy-7,8-dimethylquinoline-4-carboxylic acid; 8-ethyl-2-(1-tolylcyclopropyl)-3-hydroxyquinoline-4-carboxylic acid; 8-methyl-2-(1-p-tolylcyclopropyl)-3-hydroxyquinoline-4-carboxylic acid; 3-hydroxy-6,8-dimethyl-2-(1-p-tolylcyclopropyl)quinoline-4-carboxylic acid; 8-(1,1,1,3,3,3-hexafluoro-2-hydroxypropan-2-yl)-3-hydroxy-2-(1-p-tolylcyclopropyl)quinoline-4-carboxylic acid; 3-hydroxy-8-isopropyl-2-(1-p-tolylcyclopropyl)quinoline-4-carboxylic acid; 8-ethyl-3-hydroxy-2-(1-(4-(trifluoromethyl)phenyl)cyclopropyl)quinoline-4-carboxylic acid; 3-hydroxy-8-isopropyl-2-(1-(4-(trifluoromethyl)phenyl)cyclopropyl)quinoline-4-carboxylic acid; 7-ethyl-3-hydroxy-2-(1-(4-(trifluoromethyl)phenyl)cyclopropyl)quinoline-4-carboxylic acid; and 3-hydroxy-6-(trifluoromethoxy)-2-(1-(4-(trifluoromethyl)phenyl)cyclopropyl)quinoline-4-carboxylic acid, or a pharmaceutically acceptable salt, hydrate, or ester thereof.

36. A compound of claim 1 wherein the compound is selected from 3-hydroxy-8-(thiophen-3-yl)-2-(1-(4-(trifluoromethyl)phenyl)cyclopropyl)quinoline-4-carboxylic acid; 3-hydroxy-8-phenyl-2-(1-(4-(trifluoromethyl)phenyl)cyclopropyl)quinoline-4-carboxylic acid; 3-hydroxy-2-(1-(4-(trifluoromethyl)phenyl)cyclopropyl)-7,8,9,10-tetrahydrobenzo[h]quinoline-4-carboxylic acid; 2-(1-(4-chlorophenyl)cyclopropyl)-3-hydroxy-6-methyl-8-(trifluoromethyl)quinoline-4-carboxylic acid; 6-chloro-2-(1-(4-chlorophenyl)cyclopropyl)-3-hydroxy-8-(trifluoromethyl)quinoline-4-carboxylic acid; 2-(1-(4-chlorophenyl)cyclopropyl)-3-hydroxy-6-phenyl-8-(trifluoromethyl)quinoline-4-carboxylic acid; 2-(1-(4-chlorophenyl)cyclopropyl)-3-hydroxy-8-methyl-6-(trifluoromethyl)quinoline-4-carboxylic acid; 2-(1-(4-chlorophenyl)cyclopropyl-6-ethyl-3-hydroxy-8-(trifluoromethyl)quinoline-4-carboxylic acid; 2-(1-(4-chlorophenyl)cyclopropyl)-8-ethyl-3-hydroxy-6-(trifluoromethyl)quinoline-4-carboxylic acid; and 2-(1-(4-chlorophenyl)cyclopropyl)-3-hydroxy-8-phenyl-6-(trifluoromethyl)quinoline-4-carboxylic acid, or a pharmaceutically acceptable salt, hydrate, or ester thereof.

37. A compound of claim 1 wherein the compound is selected from 3-hydroxy-6-methyl-2-(1-phenylcyclopropyl)-8-(trifluoromethyl)quinoline-4-carboxylic acid; 3-hydroxy-6-phenyl-2-(1-phenylcyclopropyl)-8-(trifluoromethyl)quinoline-4-carboxylic acid; 6-bromo-2-(1-(4-chlorophenyl)cyclopropyl)-3-hydroxy-8-(trifluoromethyl)quinoline-4-carboxylic acid; 6-ethyl-3-hydroxy-2-(1-phenylcyclopropyl)-8-(trifluoromethyl)quinoline-4-carboxylic acid; 3-hydroxy-2-(1-(4-chlorophenyl)cyclopropyl)-6,8-bis(trifluoromethyl)quinoline-4-carboxylic acid; 2-(1-(4-phenyl)cyclopropyl-3-hydroxy-6,8-bis-(trifluoromethyl)quinoline-4-carboxylic acid; 6-bromo-3-hydroxy-2-(1-phenylcyclopropyl)-8-(trifluoromethyl)quinoline-4-carboxylic acid; 2-(1-(4-chlorophenyl)cyclopropyl)-3-hydroxyquinoline-4,8-dicarboxylic acid; 2-(1-(4-chloro-phenyl)-cyclopropyl)-8-cyclopropyl-3-hydroxy-quinoline-4-carboxylic acid; 8-cyclopropyl-3-hydroxy-2-(1-phenyl-cyclopropyl)-quinoline-4-carboxylic acid; 3-hydroxy-2-(1-phenyl-cyclopropylmethyl)-8-trifluoromethyl-quinoline-4-carboxylic acid; 2-(1-benzyl-cyclopropyl)-3-hydroxy-8-trifluoromethyl-quinoline-4-carboxylic acid; and 3-hydroxy-7,8-dimethyl-2-(1-p-tolyl-cyclopropyl)-quinoline-4-carboxylic acid, or a pharmaceutically acceptable salt, hydrate, or ester thereof.

38. A compound selected from 3-hydroxy-2-(2-phenylpropan-2-yl)-7,8,9,10-tetrahydrobenzo[h]quinoline-4-carboxylic acid; 3-hydroxy-7,8-dimethyl-2-(2-phenylpropan-2-yl)quinoline-4-carboxylic acid; 3-hydroxy-8-isopropyl-2-(2-phenylpropan-2-yl)quinoline-4-carboxylic acid; 3-hydroxy-2-(2-phenylpropan-2-yl)-8-(trifluoromethyl)quinoline-4-carboxylic acid; 2-(2-(4-chlorophenyl)propan-2-yl)-3-hydroxy-8-isopropylquinoline-4-carboxylic acid; 2-(2-(4-chlorophenyl)propan-2-yl)-3-hydroxy-8-(trifluoromethyl)quinoline-4-carboxylic acid; 2-(2-(4-chlorophenyl)propan-2-yl)-3-hydroxy-7,8,9,10-tetrahydrobenzo[h]quinoline-4-carboxylic acid; 2-(2-(4-chlorophenyl)propan-2-yl)-3-hydroxy-7,8-dimethylquinoline-4-carboxylic acid; 2-(2-(4-chlorophenyl)propan-2-yl)-8-(1,1,1,3,3,3-hexafluoro-2-hydroxypropan-2-yl)-3-hydroxyquinoline-4-carboxylic acid; 3-hydroxy-2-(1-phenylethyl)-8-(trifluoromethyl)quinoline-4-carboxylic acid; 2-[1-(4-chlorophenyl)ethyl]-3-hydroxy-7,8,9,10-tetrahydrobenzo[h]quinoline-4-carboxylic acid; 3-hydroxy-2-(1-phenylethyl)-7,8,9,10-tetrahydrobenzo[h]quinoline-4-carboxylic acid; 3-hydroxy-2-(1-phenylpropyl)-8-(trifluoromethyl)quinoline-4-carboxylic acid; 3-hydroxy-7,8-dimethyl-2-(1-phenylpropyl)quinoline-4-carboxylic acid; 3-hydroxy-2-(2-methyl-1-phenylpropyl)-8-(trifluoromethyl)quinoline-4-carboxylic acid; 3-hydroxy-8-isopropyl-2-(2-methyl-1-phenylpropyl)quinoline-4-carboxylic acid; 3-hydroxy-7,8-dimethyl-2-(2-methyl-1-phenylpropyl)quinoline-4-carboxylic acid; 3-hydroxy-2-(1-phenylpropan-2-yl)-8-(trifluoromethyl)quinoline-4-carboxylic acid; 3-hydroxy-8-isopropyl-2-(1-phenylpropan-2-yl)quinoline-4-carboxylic acid; 3-hydroxy-7,8-dimethyl-2-(1-phenylpropan-2-yl)quinoline-4-carboxylic acid; 3-hydroxy-2-(2-phenylpropyl)-8-(trifluoromethyl)quinoline-4-carboxylic acid; 3-hydroxy-8-isopropyl-2-(2-phenylpropyl)quinoline-4-carboxylic acid; 3-hydroxy-7,8-dimethyl-2-(2-phenylpropyl)quinoline-4-carboxylic acid; and 2-(4-chlorobenzyl)-3-[(morpholin-4-ylcarbonyl)oxy]-7,8,9,10-tetrahydrobenzo[h]quinoline-4-carboxylic acid, or a pharmaceutically acceptable salt, hydrate, or ester thereof.

39. A pharmaceutical composition comprising a therapeutically effective amount of a compound of claim 1 or a pharmaceutically acceptable salt, hydrate, or ester thereof, and a pharmaceutically acceptable carrier or excipient.

40. A method of inhibiting selectin-mediated intracellular adhesion in a mammal comprising administering to said mammal a therapeutically effective amount of a compound of claim 1 or a pharmaceutically acceptable salt, hydrate, or ester thereof.

41. A method of treating or preventing thrombosis in a mammal comprising administering to the mammal a therapeutically effective amount compound of claim 1, or a pharmaceutically acceptable salt, hydrate, or ester form thereof.

42. A method of treating or preventing a disease or disorder in a mammal, the method comprising administering to the mammal a therapeutically effective amount compound of claim 1, or a pharmaceutically acceptable salt, hydrate, or ester form thereof, wherein the disease or disorder is selected from atherosclerosis, atherothrombosis, restenosis, myocardial infarction, ischemia reperfusion, Reynauld's syndrome, inflammatory bowel disease, osteoarthritis, acute respiratory distress syndrome, asthma, chronic obstructive pulmonary disease (COPD), emphysema, lung inflammation, delayed type hypersensitivity reaction, idiopathic pulmonary fibrosis, cystic fibrosis, thermal injury, stroke, experimental allergic encephalomyelitis, multiple organ injury syndrome secondary to trauma, neutrophilic dermatosis (Sweet's disease), glomerulonephritis, ulcerative colitis, Crohn's disease, necrotizing enterocolitis, cytokine-induced toxicity, gingivitis, periodontitis, hemolytic uremic syndrome, psoriasis, systemic lupus erythematosus, autoimmune thyroiditis, multiple sclerosis, rheumatoid arthritis, scleritis, Grave's disease, immunological-mediated side effects of treatment associated with hemodialysis or leukapheresis, granulocyte transfusion associated syndrome, deep vein thrombosis, post-thrombitic syndrome, unstable angina, transient ischemic attacks, peripheral vascular disease, metastasis associated with cancer, sickle cell anemia, organ transplant rejection and congestive heart failure.
